(19) United States
(12) Patent Application Publication
(10) Pub. No.: US 2017/0209345 A1
(43) Pub. Date: Jul. 27, 2017

(54) DENTAL PROSTHESIS

(71) Applicant: MITSUI CHEMICALS, INC., Minato-ku, Tokyo (JP)

(72) Inventors: Akira HASEGAWA, Chiba-shi, Chiba (JP); Yasufumi TSUCHIYA, Funabashi-shi, Chiba (JP); Koya KOJIMA, Urayasu-shi, Chiba (JP); Koju OKAZAKI, Chiba-shi, Chiba (JP); Yohsuke ASANO, Chiba-shi, Chiba (JP); Kenichi FUJII, Yokohama-shi, Kanagawa (JP); Takaaki HAYASHI, Ichihara-shi, Chiba (JP)

(73) Assignee: MITSUI CHEMICALS, INC., Minato-ku, Tokyo (JP)

(21) Appl. No.: 15/324,108

(22) PCT Filed: Jul. 8, 2015
(86) PCT No.: PCT/JP2015/069699

§ 371 Date: Jan. 5, 2017

(30) Foreign Application Priority Data
Jul. 11, 2014 (JP) 2014-143677

(51) Int. Cl. A61K 6/087; A61C 5/77; A61C 8/00; A61C 13/00

(52) U.S. Cl. CPC A61K 6/087; A61C 13/0012; A61C 5/77; A61C 8/0013

(57) ABSTRACT

An object of the present invention is to provide a dental material, particularly, a dental prosthesis, having excellent properties such as hydrophilicity and antifouling properties. The dental prosthesis according to the present invention includes a monolayer film obtained by curing a composition including: a compound (I) having at least one hydrophilic group selected from anionic hydrophilic groups and cationic hydrophilic groups, and at least one functional group with a polymerizable carbon-carbon double bond; a compound (II) having two or more functional groups with a polymerizable carbon-carbon double bond (wherein the compound (II) has no anionic hydrophilic group nor cationic hydrophilic group); and a surfactant (III) having a hydrophilic moiety including an anionic hydrophilic group, a cationic hydrophilic group or two or more hydroxyl groups, and a hydrophobic moiety composed of an organic residue (wherein the surfactant has no polymerizable carbon-carbon double bond).

### TECHNICAL FIELD

The present invention relates to a dental prosthesis.

### BACKGROUND ART

In recent years, there have been increasing demands that substrates made of organic materials such as plastics and inorganic materials such as glass be improved in terms of antifogging properties and antifouling properties.

To solve the fogging problems, a method has been proposed in which an antifogging coating liquid containing a reactive surfactant and an acrylic oligomer is applied to provide enhanced hydrophilicity and water absorption (see, for example, Non Patent Document 1). The fouling problems have been addressed by methods in which the hydrophilicity of the surface of materials is enhanced so that fouling such as airborne hydrophobic substances which have become attached to surfaces such as exterior walls can be detached and removed from the surfaces by water spray or rainfall (see, for example, Non Patent Documents 2 and 3).

Further, hydrophilic materials have been proposed in which a monomer composition which is capable of cross-linking polymerization is applied onto the surface of a substrate and is incompletely polymerized while controlling the UV dose to form a crosslinked polymer, and subsequently a hydrophilic monomer is applied and UV rays are applied again to block- or graft-polymerize the hydrophilic monomer to the surface of the crosslinked polymer (Patent Document 1 and Patent Document 2).

However, this simple block or graft polymerization of a hydrophilic monomer to the surface of a substrate attaches the hydrophilic groups only to the surface, resulting in low durability. Thus, the hydrophilic materials have drawbacks in that they cannot withstand long-term use.

To solve the above problem, the present inventors have proposed monolayer films in which specific anionic hydrophilic groups have a concentration gradient (an uneven distribution) between the inside of the film and the film surface and the anionic hydrophilic groups are present in a higher concentration near the surface (Patent Document 3 and Patent Document 4).

On the other hand, Patent Document 5 describes a dental polymerizable composition including: a fluorine compound composed of a chain polymer having a main chain containing a monomer unit having a hydrophilic group, and having terminal groups, each containing a fluoroalkyl group, attached at both the terminals of the main chain; a polymerizable monomer; and a polymerization initiator.

### CITATION LIST

### Patent Documents

Patent Document 1: JP 2001-98007 A
Patent Document 2: JP 2011-229734 A
Patent Document 3: WO 2007/064003
Patent Document 4: WO 2012/014829
Patent Document 5: JP 4673310 B

### Non-Patent Documents

Non Patent Document 1: TREND, annual research report by TOAGOSEI CO., LTD., 1999, February issue, pp. 39-44
Non Patent Document 2: Koubunshi (Polymers), 44(5), p. 307
Non Patent Document 3: Mirai Zairyou (Future materials), 2(1), pp. 36-41

### SUMMARY OF THE INVENTION

### Problems to be Solved by the Invention

An object of the present invention is to provide a dental material, particularly, a dental prosthesis, having excellent properties such as hydrophilicity and antifouling properties.

### Means for Solving the Problems

The present inventors have made extensive studies directed to solving the above mentioned problems. As a result, the present inventors have found that it is possible to obtain a cured product, particularly, a monolayer film, which has excellent properties such as hydrophilicity and antifouling properties and which is suitable as a dental material such a dental prosthesis, from a composition including: a compound containing a specific hydrophilic group and a functional group with a polymerizable carbon-carbon double bond; a compound having two or more functional groups with a polymerizable carbon-carbon double bond; and a specific surfactant. At the same time, the inventors also discovered that the use of such a monolayer film allows for producing a dental prosthesis having excellent properties such as hydrophilicity and antifouling properties. The present invention has been completed based on these findings.

In other words, the present invention relates to the following items [1] to [9].

[1]

A dental prosthesis comprising a monolayer film obtained by curing a composition comprising:

a compound (I) having at least one hydrophilic group selected from anionic hydrophilic groups and cationic hydrophilic groups, and at least one functional group with a polymerizable carbon-carbon double bond;

a compound (II) having two or more functional groups with a polymerizable carbon-carbon double bond (wherein the compound (II) has no anionic hydrophilic group nor cationic hydrophilic group); and

a surfactant (III) having a hydrophilic moiety including an anionic hydrophilic group, a cationic hydrophilic group or two or more hydroxyl groups, and a hydrophobic moiety composed of an organic residue (wherein the surfactant has no polymerizable carbon-carbon double bond).

[2]

The dental prosthesis according to item [1], wherein the monolayer film has a concentration gradient (Sa/Da) of at least one type of hydrophilic groups selected from anionic hydrophilic groups, cationic hydrophilic groups and hydroxyl group, of 1.1 or more,

wherein the concentration gradient (Sa/Da) is obtained from:
the concentration (Sa) at the surface of the monolayer film; and
the concentration (Da) at ½ point in the thickness of the monolayer film.

[3]

The dental prosthesis according to item [1] or [2] wherein the monolayer film has a water contact angle of 30° or less.

[4]

The dental prosthesis according to any of items [1] to [3], wherein the monolayer film has a film thickness of 0.1 to 100 μm.

[5]

The dental prosthesis according to any of items [1] to [4], wherein the monolayer film is obtained by: coating a composition comprising the compound (I), the compound (II), the surfactant (III) and a solvent on a substrate; then removing the solvent; and then subjecting the resultant to curing.

[6]

The dental prosthesis according to item [5], wherein the coating step is carried out by a dip method.

[7]

The dental prosthesis according to any of items [1] to [6], wherein the compound (I) is a compound represented by the general formula (100) below:

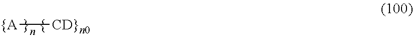

(wherein in the formula (100),

A represents a C₂₋₁₀₀ organic group having 1 to 5 functional groups with a polymerizable carbon-carbon double bond;

CD represents a group containing at least one hydrophilic group, selected from those groups represented by the general formulas (101), (102) and (112) below;

n represents the number of As bound to CD and is 1 or 2; and

n0 represents the number of CDs bound to A and is an integer of 1 to 5);

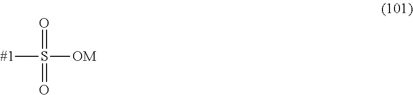

(wherein in the formula (101), M represents a hydrogen atom, an alkali metal, an alkaline earth metal (½ atom) or an ammonium ion; and #1 indicates a hand bound to a carbon atom present in A in the formula (100));

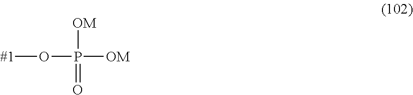

(wherein in the formula (102), M represents a hydrogen atom, an alkali metal, an alkaline earth metal (½ atom) or an ammonium ion; and #1 indicates a hand bound to a carbon atom present in A in the formula (100)); and

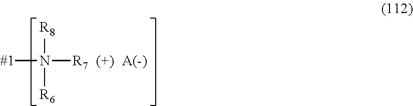

(wherein in the formula (112), A(−) represents a halogen ion, a formate ion, an acetate ion, a sulfate ion, a hydrogen sulfate ion, a phosphate ion or a hydrogen phosphate ion; R₆ to R₈ each independently represent a hydrogen atom, a C₁₋₂₀ alkyl, alkylaryl, alkylbenzyl, alkylcycloalkyl, alkylcycloalkylmethyl or cycloalkyl group, a phenyl group or a benzyl group; and #1 indicates a hand bound to a carbon atom present in A in the formula (100)).

[8]

The dental prosthesis according to item [7], wherein A in the general formula (100) is at least one functional group selected from those groups represented by the general formulas (120), (123) and (124) below:

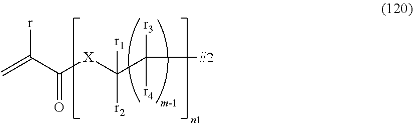

(wherein in the formula (120), X represents —O—, —S—, —NH— or —NCH₃—; r represents a hydrogen atom or a methyl group; r₁ to r₄ each independently represent a hydrogen atom, a methyl group, an ethyl group or a hydroxyl group; m1 is an integer of 0 to 10; n1 is an integer of 0 to 100; and #2 indicates a hand bound to #1 present in at least one group selected from those groups represented by the general formulas (101), (102) and (112));

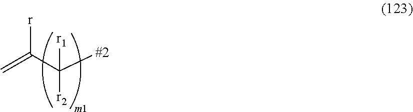

(wherein in the formula (123), r represents a hydrogen atom or a methyl group; r₁ and r₂ each independently represent a hydrogen atom, a methyl group, an ethyl group or a hydroxyl group; m1 is an integer of 0 to 10; and #2 indicates a hand bound to #1 present in at least one group selected from those groups represented by the general formulas (101), (102) and (112)); and

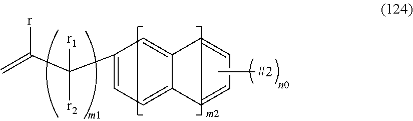

(wherein in the formula (124), r represents a hydrogen atom or a methyl group; r₁ and r₂ each independently represent a hydrogen atom, a methyl group, an ethyl group or a hydroxyl group; m1 is an integer of 0 to 10; m2 is an integer of 0 to 5; n0 is an integer of 1 to 5; and #2 indicates a hand bound to #1 present in at least one group selected from those groups represented by the general formulas (101), (102) and (112)).

[9]

The dental prosthesis according to any of items [1] to [8], wherein the surfactant is a compound represented by the general formula (300) below:

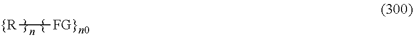

(wherein in the formula (300),

R represents a C₄₋₁₀₀ organic residue,

FG represents a group containing at least one hydrophilic group, selected from those groups represented by the general formulas (301), (302), (312) and (318) below;

n represents the number of Rs bound to FG and is 1 or 2; and

n0 represents the number of FGs bound to R and is an integer of 1 to 5; and when FG is a group including one hydroxyl group, n0 is an integer of 2 to 5);

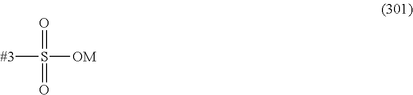

(wherein in the formula (301), M represents a hydrogen atom, an alkali metal, an alkaline earth metal (½ atom) or an ammonium ion; and #3 indicates a hand bound to a carbon atom present in R in the formula (300));

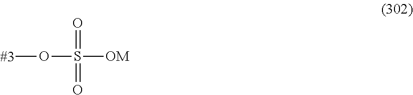

(wherein in the formula (302), M represents a hydrogen atom, an alkali metal, an alkaline earth metal (½ atom) or an ammonium ion; and #3 indicates a hand bound to a carbon atom present in R in the formula (300));

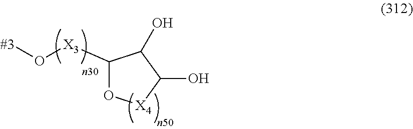

(wherein in the formula (312), X₃ and X₄ each independently represent —CH₂—, —CH(OH)— or —CO—; n₃₀ is an integer of 0 to 3; n₅₀ is an integer of 0 to 5; when n₃₀ is 2 or greater, X₃s may be the same as or different from one another; when n₅₀ is 2 or greater, X₄s may be the same as or different from one another; and #3 indicates a hand bound to a carbon atom present in R in the formula (300)); and

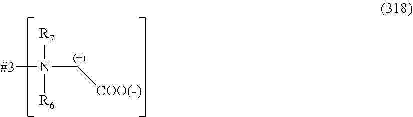

(wherein in the formula (318), R₆ and R₇ each independently represent a hydrogen atom, a C₁₋₂₀ alkyl, alkylaryl, alkylbenzyl, alkylcycloalkyl, alkylcycloalkylmethyl or cycloalkyl group, a phenyl group or a benzyl group; and #3 indicates a hand bound to a carbon atom present in R in the formula (300)).

### Effect of the Invention

The composition of the invention serves to provide a cured product, particularly a monolayer film, which has excellent properties such as hydrophilicity and antifouling properties, and which is useful as a dental material, for example, a dental prosthesis. A dental prosthesis including such a monolayer film has excellent properties such as hydrophilicity and antifouling properties.

### BRIEF DESCRIPTION OF THE DRAWINGS

FIG. 1 is a schematic view illustrating a method of preparing a sample for the measurement of the gradient of the concentration of hydrophilic groups (anion concentration) (Sa/Da) in Examples.

FIG. 2 is a schematic view illustrating a method for removing a solvent from a polymerizable composition in Examples.

### MODE FOR CARRYING OUT THE INVENTION

The present invention will now be described.

### [Composition]

The composition used in the present invention includes the following compound (I), compound (II), and surfactant (III). In the present specification, the composition may be referred to as “the dental composition of the invention” or “the composition of the invention”, for convenience of description.

### <Compound (I)>

The compound (I) included in the dental composition of the invention has at least one hydrophilic group selected from anionic hydrophilic groups and cationic hydrophilic groups, and at least one functional group with a polymerizable carbon-carbon double bond. In other words, in the present invention, the compound (I) necessarily contains, as a hydrophilic group(s), an anionic hydrophilic group or a cationic hydrophilic group, or alternatively, both an anionic hydrophilic group and a cationic hydrophilic group. By copolymerizing the composition including the compound containing such a hydrophilic group and a functional group with a carbon-carbon double bond, it is possible to impart hydrophilicity to the resulting cured product, and to obtain a dental prosthesis having an excellent hydrophilicity. The compound (I) may or may not contain a hydroxyl group as the hydrophilic group(s), in addition to an anionic hydrophilic group and/or cationic hydrophilic group.

### Hydrophilic Groups

Examples of the anionic hydrophilic groups include sulfo group, carboxyl group, phosphate group, O-sulfate group (—O—SO₃⁻) and N-sulfate group (—NH—SO₃⁻). Of the anionic hydrophilic groups, sulfo group, carboxyl group, and phosphate group are preferred. In the present invention, sulfo group and phosphate group are particularly preferred among these anionic hydrophilic groups.

In the compound (I), the anionic hydrophilic groups may be in the form of a free acid, or in the form of a salt with an appropriate cation.

Thus, the sulfo group, the carboxyl group and the phosphate group may be typically present in the compound (I) in the forms of (α), (β), and (γ1) or (γ2), respectively, represented by the following formulas. In the present invention, when the compound (I) contains a phosphate group, it is preferred that the phosphate group be contained in the compound (I) in the form represented by the following formula (γ1).

—SO₃Z (α)

—COOZ (β)

—OP═O(OZ)₂ (γ1)

(—O)₂P═O(OZ)₁ (γ2)

In each of the formulas (α) to (γ2), Z is at least one cation selected from the group consisting of a hydrogen ion, an ammonium ion, an alkali metal ion, and an alkaline earth metal (½ atom) ion.

In the invention, the ammonium ion is a cation resulting from the bonding of a hydrogen ion to ammonia, a primary amine, a secondary amine or a tertiary amine. From the point of view of hydrophilicity, the ammonium ion is preferably a cation in which a hydrogen ion is bonded to ammonia or an amine having a small number of carbon atoms, and is more preferably an ammonium ion formed by the bonding of a hydrogen ion to ammonia, or methylammonium.

The alkali metal as used in the present invention refers to a metal of Group 1 in the periodic table, and examples thereof include lithium, sodium, potassium, and rubidium.

The alkaline earth metal as used in the present invention refers to a metal of Group 2 in the periodic table, and examples thereof include beryllium, magnesium, calcium, strontium, and barium.

Of the cations which can be the above descried Z, alkali metal ions are preferred, and sodium ion, potassium ion and rubidium ion are more preferred.

Examples of the cationic hydrophilic groups include quaternary ammonium groups, betaine groups and amine oxide groups. Of these cationic hydrophilic groups, quaternary ammonium groups and betaine groups are preferred. In the present invention, quaternary ammonium groups are particularly preferred.

The hydroxyl group may be an alcoholic hydroxyl group or a phenolic hydroxyl group as long as the effect of the invention is obtained. However, an alcoholic hydroxyl group is preferred. Some of the above mentioned anionic hydrophilic groups may include a partial structure formally represented as “—OH”, such as sulfo group, phosphate group and carboxyl group. In the present invention, however, the “—OH” which is a part of the anionic hydrophilic group is not regarded as a “hydroxyl group”.

The hydrophilic groups present in the compounds (I) are preferably anionic hydrophilic groups.

When the compound (I) has two or more hydrophilic groups, these hydrophilic groups may be the same as or different from one another.

Functional Groups with a Polymerizable Carbon-Carbon Double Bond

The functional groups with a polymerizable carbon-carbon double bond are not particularly limited as long as the functional groups can initiate radical polymerization or ion polymerization. Examples thereof include acryloyl group, methacryloyl group, acryloyloxy group, methacryloyloxy group, acryloylthio group, methacryloylthio group, acrylamide group, methacrylamide group, allyl group, vinyl group, isopropenyl group, maleyl group (—CO—CH═CH—CO—), itaconyl group and styryl group. In the present specification, acryloyl and methacryloyl may be collectively referred to as (meth)acryloyl, acryloyloxy and methacryloyloxy as (meth)acryloyloxy, acryloylthio and methacryloylthio as (meth)acryloylthio, and acrylamide and methacrylamide as (meth)acrylamide.

When the compound (I) has two or more “functional groups with a polymerizable carbon-carbon double bond”, these functional groups may be the same as or different from one another.

### Preferred Embodiment of the Compound (I)

The compound (I) used in the present invention is a compound having the above described hydrophilic group(s) and the above described functional group (s) with a polymerizable carbon-carbon double bond, and the numbers of the “hydrophilic group(s)” and “functional group(s) with a polymerizable carbon-carbon double bond” included in the compound (I) may each be one, or more than one.

In the present invention, the compound (I) is preferably a compound represented by the general formula (100) below:

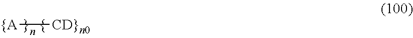

In the formula (100),

A represents a C₂₋₁₀₀ organic group having 1 to 5 functional groups with a polymerizable carbon-carbon double bond;

CD represents a group containing at least one hydrophilic group, selected from those groups represented by the general formulas (101), (102) and (112) below;

n represents the number of As bound to CD and is 1 or 2; and

n0 represents the number of CDs bound to A and is an integer of from 1 to 5.

Examples of the group CD containing an anionic hydrophilic group include hydrophilic groups represented by the general formulas (101) and (102) below.

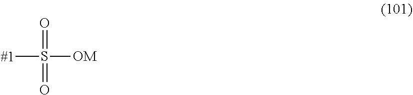

In the formula (101), M is a hydrogen atom, an alkali metal, an alkaline earth metal (½ atom) or an ammonium ion; and #1 indicates a hand bonded to a carbon atom present in A in the formula (100).

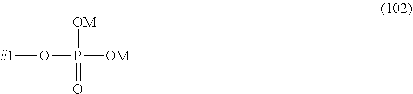

In the formula (102), M at each occurrence is a hydrogen atom, an alkali metal, an alkaline earth metal (½ atom) or an ammonium ion; and #1 indicates a hand bonded to a carbon atom present in A in the formula (100).

Examples of the group CD containing a cationic hydrophilic group include hydrophilic groups represented by the general formula (112) below.

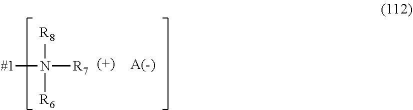

In the formula (112), A(−) represents a halogen ion, a formate ion, an acetate ion, a sulfate ion, a hydrogen sulfate ion, a phosphate ion or a hydrogen phosphate ion; R₆ to R₈ each independently represent a hydrogen atom, a C₁₋₂₀ alkyl, alkylaryl, alkylbenzyl, alkylcycloalkyl, alkylcycloalkylmethyl or cycloalkyl group, a phenyl group or a benzyl group; and #1 indicates a hand bound to a carbon atom present in A in the formula (100).

In the general formula (100), A is preferably at least one functional group with a polymerizable carbon-carbon double bond selected from those groups represented by the general formulas (120), (123) and (124) below. Of these, a C₂₋₁₀₀ organic group is more preferred. In other words, the functional group preferably used as A is at least one selected from those groups represented by the general formulas (120), (123) and (124) below.

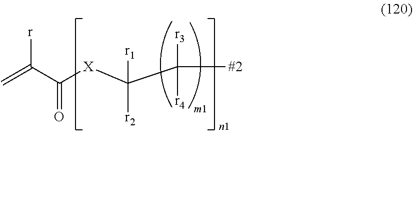

In the formula (120), X represents —O—, —S—, —NH— or —NCH₃—; r represents a hydrogen atom or a methyl group; r₁ to r₄ each independently represent a hydrogen atom, a methyl group, an ethyl group or a hydroxyl group; m1 is an integer of 0 to 10; n1 is an integer of 0 to 100; and #2 indicates a hand bound to #1 present in at least one group selected from those groups represented by the general formulas (101), (102) and (112).

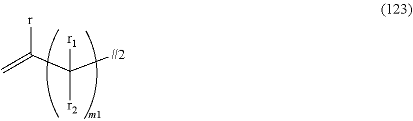

In the formula (123), r represents a hydrogen atom or a methyl group; r₁ and r₂ each independently represent a hydrogen atom, a methyl group, an ethyl group or a hydroxyl group; m1 is an integer of 0 to 10; and #2 indicates a hand bound to #1 present in at least one group selected from those groups represented by the general formulas (101), (102) and (112).

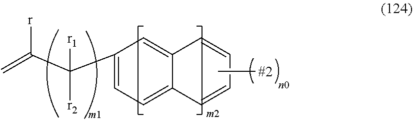

In the formula (124), r represents a hydrogen atom or a methyl group; r₁ and r₂ each independently represent a hydrogen atom, a methyl group, an ethyl group or a hydroxyl group; m1 is an integer of 0 to 10; m2 represents an integer of 0 to 5; n0 is an integer of 1 to 5; and #2 indicates a hand bound to #1 present in at least one group selected from those groups represented by the general formulas (101), (102) and (112)).

The compound (I) including an anionic hydrophilic group is preferably a compound represented by any of the general formulas (Ia), (Ic), (Id) and (Il) below.

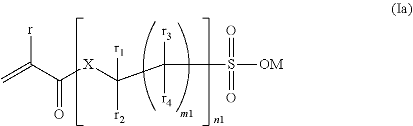

In the formula (Ia), X represents —O—, —S—, —NH— or —NCH₃—; r represents a hydrogen atom or a methyl group; r₁ to r₄ each independently represent a hydrogen atom, a methyl group, an ethyl group or a hydroxyl group; m1 is an integer of 0 to 10; n1 is an integer of 0 to 100; and M represents a hydrogen ion, an ammonium ion, an alkali metal ion or an alkaline earth metal ion (½ atom).

Examples of the compound represented by the general formula (Ia) include 1-(meth)acryloyloxymethylsulfonic acid, 2-(meth)acryloyloxyethylsulfonic acid, 2-(meth)acryloylthioethylsulfonic acid, 3-(meth)acryloyloxypropylsulfonic acid, 2-(meth)acryloyloxypropylsulfonic acid, 3-(meth)acryloyloxy-2-hydroxypropyl-1-sulfonic acid, 4-(meth)acryloyloxybutylsulfonic acid, 5-(meth)acryloyloxy-3-oxapentylsulfonic acid, 5-(meth)acryloyloxy-3-thiapentylsulfonic acid, 6-(meth)acryloyloxyhexylsulfonic acid, 8-(meth)acryloyloxy-3,6-dioxaoctylsulfonic acid, (meth)acrylamidomethylsulfonic acid, (meth)acrylthiomethylsulfonic acid, 3-(meth)acrylthiopropylsulfonic acid, 2-(meth)acrylamidoethylsulfonic acid, 2-(meth)acrylamido-N-methyl-ethylsulfonic acid, 3-(meth)acrylamidopropyl-1-sulfonic acid, 2-(meth)acrylamidopropyl-1-sulfonic acid, and 2-(meth)acrylamido-2-methyl-propanesulfonic acid ((meth)acrylamido-t-butylsulfonic acid); and salts thereof such as lithium salts, sodium salts, potassium salts, rubidium salts, ammonium salts, magnesium salts and calcium salts; and so on.

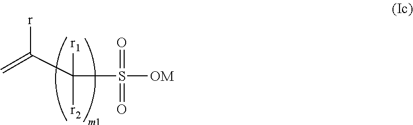

In the formula (Ic), r represents a hydrogen atom or a methyl group; r₁ and r₂ each independently represent a hydrogen atom, a methyl group, an ethyl group or a hydroxyl group; m1 is an integer of 0 to 10; N represents a hydrogen ion, an ammonium ion, an alkali metal ion or an alkaline earth metal (½ atom) ion.

Examples of the compound represented by the general formula (Ic) include vinylsulfonic acid, isopropenylsulfonic acid, allylsulfonic acid, methallylsulfonic acid and 5,6-hexenyl-1-sulfonic acid; and salts thereof such as lithium salts, sodium salts, potassium salts, rubidium salts, ammonium salts, magnesium salts and calcium salts, and so on.

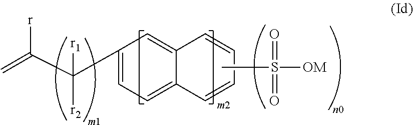

In the formula (Id), r represents a hydrogen atom or a methyl group; r₁ and r₂ each independently represent a hydrogen atom, a methyl group, an ethyl group or a hydroxyl group; m1 is an integer of 0 to 10; m2 is an integer of 0 to 5; n0 is an integer of 1 to 5; M represents a hydrogen ion, an ammonium ion, an alkali metal ion or an alkaline earth metal (½ atom) ion.

Examples of the compound represented by the general formula (Id) include:

styrene sulfonic acid, isopropenylbenzenesulfonic acid, allylbenzenesulfonic acid, methallylbenzenesulfonic acid, vinylnaphthalenesulfonic acid, isopropenylnaphthalenesulfonic acid, allylnaphthalenesulfonic acid, methallylnaphthalenesulfonic acid, vinylanthracenesulfonic acid, isopropenylanthracenesulfonic acid, allylanthracenesulfonic acid, methallylanthracenesulfonic acid, vinylphenanthrenesulfonic acid, isopropenylphenanthrenesulfonic acid, allylphenanthrenesulfonic acid, and methallylphenanthrenesulfonic acid; and salts thereof such as lithium salts, sodium salts, potassium salts, rubidium salts, ammonium salts, magnesium salts and calcium salts;

styrenedisulfonic acid; and salts thereof such as dilithium salt, disodium salt, dipotassium salt, dirubidium salt, diammonium salt, magnesium salt and calcium salt;

isopropenylbenzenedisulfonic acid; and salts thereof such as lithium salt, sodium salt, potassium salt, rubidium salt, ammonium salt, magnesium salt and calcium salt;

vinylnaphthalenetrisulfonic acid; and salts thereof such as trilithium salt, trisodium salt, tripotassium salt, trirubidium salt, triammonium salt, magnesium salt and calcium salt; and

isopropenylnaphthalenetrisulfonic acid; and salts thereof such as dilithium salt, disodium salt, dipotassium salt, dirubidium salt, diammonium salt, magnesium salt and calcium salt; and so on.

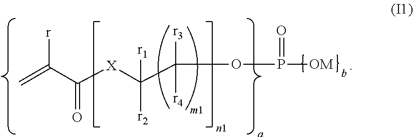

In the formula (Il), X represents —O—, —S—, —NH— or —NCH₃—; r represents a hydrogen atom or a methyl group; r₁ to r₄ each independently represent a hydrogen atom, a methyl group, an ethyl group or a hydroxyl group; m1 is an integer of 0 to 10; n1 is an integer of 0 to 100; and M represents a hydrogen ion, an ammonium ion, an alkali metal ion or an alkaline earth metal (½ atom) ion. a is 1 and b is 2; and Ms may be the same as or different from each other.

Examples of the compound represented by the general formula (Il) include:

(meth)acryloyloxymethyl phosphoric acid, 2-(meth)acryloyloxy-ethyl phosphoric acid, 2-(meth)acryloyloxy-propyl phosphoric acid, 3-(meth)acryloyloxy-propyl phosphoric acid, 4-(meth)acryloyloxy-butyl phosphoric acid, 6-(meth)acryloyloxy-hexyl phosphoric acid, 5-(meth)acryloyloxy-3-oxapentyl phosphoric acid, and 8-(meth)acryloyloxy-3,6-dioxaoctyl phosphoric acid; and salts thereof such as lithium salts, dilithium salts, sodium salts, disodium salts, potassium salts, dipotassium salts, ammonium salts, diammonium salts, magnesium salts and calcium salts; and so on.

The compound (I) having a cationic hydrophilic group is preferably a compound represented by the general formula (Ir) below.

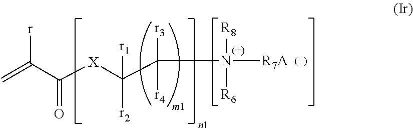

In the formula (Ir), X represents —O—, —S—, —NH— or —NCH₃—; r represents a hydrogen atom or a methyl group; r₁ to r₄ each independently represent a hydrogen atom, a methyl group, an ethyl group or a hydroxyl group; m1 is an integer of 0 to 10; n1 is an integer of 0 to 100; when n1 is 2 or greater, r₁s to r₄s, and Xs each may be the same as or different from one another; A(−) represents a halogen ion, a formate ion, an acetate ion, a sulfate ion, a hydrogen sulfate ion, a phosphate ion or a hydrogen phosphate ion; and R₆ to R₈ each independently represent a hydrogen atom, a C₁₋₂₀ alkyl, alkylaryl, alkylbenzyl, alkylcycloalkyl, alkylcycloalkylmethyl or cycloalkyl group, a phenyl group or a benzyl group.

Examples of the compound represented by the general formula (Ir) include hydrochloric acid salt, hydrobromic acid salt, sulfuric acid salt, formic acid salt, acetic acid salt and phosphoric acid salt of each of the following compounds:
N,N-dimethylaminoethyl(meth)acrylate,
N,N-dimethylamino-propyl-2-(meth)acrylate,
N,N-dimethylamino-propyl-3-(meth)acrylate,
N,N-dimethylamino-butyl-4-(meth)acrylate,
N,N-dimethylamino-hexyl-6-(meth)acrylate,
N,N-dimethylamino-octyl-8-(meth)acrylate,
N,N-dimethylamino-3-oxapentyl-5-(meth)acrylate,
N,N-diethylaminoethyl(meth)acrylate,
N,N-dipropylaminoethyl(meth)acrylate,
3-(meth)acryloyloxy-2-hydroxypropyl-1-triethylammonium,
N,N-dimethylaminoethyl(meth)acrylamide,
N,N-dimethylamino-propyl-2-(meth)acrylamide,
N,N-dimethylamino-propyl-3-(meth)acrylamide, and
N,N-dimethylamino-butyl-4-(meth)acrylamide; and so on.

The molecular weight of the compounds (I) is usually 72 to 18,000, preferably 72 to 3,000, and more preferably 72 to 1000.

The compounds (I) may be used singly, or two or more may be used in combination.

The composition of the invention contains the compound (I). At least some of the molecules of the compound (I) may be reacted to form an oligomer in the composition. Here, the oligomer usually contains 2 to 20 repeating units derived from the compound (I).

The compounds (I) may be produced by a known method or by a method in accordance with a known method. Alternatively, the compounds (I) may be purchased from the market.

### <Compound (II)>

The compound (II) included in the dental composition of the invention has two or more functional groups with a polymerizable carbon-carbon double bond. Unlike the compound (I), the compound (II) may include a hydroxyl group, but has no anionic hydrophilic group nor cationic hydrophilic group. The curing of the composition including such a compound allows for obtaining a cured product having a sufficient degree of crosslinking.

In the present invention, examples of the “functional group with a polymerizable carbon-carbon double bond” contained in the compound (II) include the same functional groups with a polymerizable carbon-carbon double bond as those contained in the compound (I). However, in an exemplary embodiment of the present invention, a (meth)acryloyl group is preferably used as the “functional group with a polymerizable carbon-carbon double bond” contained in the compound (II). The term (meth)acryloyl is used to collectively refer to acryloyl and methacryloyl.

Examples of the (meth)acryloyl groups include (meth)acryloyloxy groups, (meth)acryloylthio groups and (meth)acrylamide groups. Of these (meth)acryloyl groups, (meth)acryloyloxy groups and (meth)acryloylthio groups are preferable.

Preferred compounds (II) are those compounds which have one or more hydroxyl groups, and two or more (meth)acryloyl groups; those compounds which have one or more bonds selected from ether bonds and thioether bonds, and two or more (meth)acryloyl groups; those compounds which have one or more ester bonds (except ester bonds also forming moieties of (meth)acryloyl groups), and two or more (meth)acryloyl groups; those compounds which have one or more groups selected from alicyclic groups and aromatic groups, and two or more (meth)acryloyl groups; and those compounds which have one or more heterorings, and two or more (meth)acryloyl groups.

Examples of the compound (II) include ethylene glycol di(meth)acrylate, 1,2-propanediol di(meth)acrylate, 1,3-propanediol di(meth)acrylate, 1,4-butanediol di(meth)acrylate, 1,6-hexanediol di(meth)acrylate, 1,9-nonanediol di(meth)acrylate, 1,10-decanediol di(meth)acrylate, neopentyl glycol di(meth)acrylate, 2-methyl-1,8-octanediol di(meth)acrylate, 2-butyl-2-ethyl-1,3-propanediol di(meth)acrylate, 1,2-bis{3-(meth)acryloyloxy-2-hydroxy-propyloxy}ethane, 1,2-bis{3-(meth)acryloyloxy-2-hydroxy-propyloxy}propane, 1,3-bis{3-(meth)acryloyloxy-2-hydroxy-propyloxy}propane, 1,4-bis{3-(meth)acryloyloxy-2-hydroxy-propyloxy}butane, 1,6-bis{3-(meth)acryloyloxy-2-hydroxy-propyloxy}hexane; neopentyl glycol hydroxypivalic acid di(meth)acrylate; polyethylene glycol di(meth)acrylate, 1,2-polypropylene glycol di(meth)acrylate, 1,3-polypropylene glycol di(meth)acrylate, 1,4-polybutylene glycol di(meth)acrylate, polyethylene glycol-bis{3-(meth)acryloyloxy-2-hydroxy-propyl} ether, 1,2-polypropylene glycol-bis{3-(meth)acryloyloxy-2-hydroxy-propyl} ether; 1,2-polypropylene glycol-bis{(meth)acryloyl-poly(oxyethylene)} ether; 1,4-polybutylene glycol-bis{3-(meth)acryloyloxy-2-hydroxy-propyl} ether and so on.

Further, examples of the compound (II) include bis{2-(meth)acryloylthio-ethyl}sulfide, bis{5-(meth)acryloylthio-3-thiapentyl}sulfide; cyclohexanediol di(meth)acrylate, bis{(meth)acryloyloxy-methyl}cyclohexane, bis{7-(meth)acryloyloxy-2,5-dioxaheptyl}cyclohexane, bis{(meth)acryloyloxy-poly(ethyleneoxy)-methyl}cyclohexane; tricyclodecane dimethanol di(meth)acrylate; 2-propenoic acid{2-(1,1,-dimethyl-2-{(1-oxo-2-propenyl)oxy}ethyl)-5-ethyl-1,3-dioxane-5-yl}methyl ester (KAYARAD R-604, manufactured by Nippon Kayaku Co., Ltd.); N,N′,N″-tris{2-(meth)acryloyloxy-ethyl}isocyanurate; xylylenediol di(meth)acrylate, bis{7-(meth)acryloyloxy-2,5-dioxaheptyl}benzene, bis{(meth)acryloyloxy-poly(ethyleneoxy)-methyl}benzene; bisphenol A di(meth)acrylate, bis{(meth)acryloyl-oxyethyl}bisphenol A, bis{(meth)acryloyl-oxypropyl}bisphenol A, bis{(meth)acryloyl-poly(oxyethylene)}bisphenol A, bis{(meth)acryloyl-poly(oxy-1,2-propylene)}bisphenol A, bis{3-(meth)acryloyloxy-2-hydroxy-propyl}bisphenol A, bis{3-(meth)acryloyloxy-2-hydroxy-propyl-oxyethyl}bisphenol A, bis{3-(meth)acryloyloxy-2-hydroxy-propyl-oxypropyl}bisphenol A, bis{3-(meth)acryloyloxy-2-hydroxy-propyl-poly(oxyethylene)}bisphenol A, bis{3-(meth)acryloyloxy-2-hydroxy-propyl-poly(oxy-1,2-propylene)}bisphenol A; bis{(meth)acryloyl-oxyethyl-oxypropyl}bisphenol A, bis{(meth)acryloyl poly(oxyethylene)-poly(oxy-1,2-propylene)}bisphenol A; naphthalenediol di(meth)acrylate, bis{3-(meth)acryloyloxy-2-hydroxy-propyl-oxy}naphthalene; 9,9-fluorenediol di(meth)acrylate, 9,9-bis{4-(2-(meth)acryloyloxy-ethyl-oxy)}fluorene, 9,9-bis{3-phenyl-4-(meth)acryloyloxy-poly(ethyleneoxy)}fluorene; and so on.

Still further, examples of the compound (II) include phenol novolak epoxy (meth)acrylate (trade names “NK Oligo EA-6320, EA-7120 and EA-7420”, manufactured by Shin-Nakamura Chemical Co., Ltd.); glycerol-1,3-di(meth)acrylate, 1-acryloyloxy-2-hydroxy-3-methacryloyloxy-propane, 2,6,10-trihydroxy-4,8-dioxaundecane-1,11-di(meth)acrylate, 1,3-bis{3-(meth)acryloyloxy-2-hydroxy-propyl-oxy}-2-hydroxypropane, 1,2,3-tris{3-(meth)acryloyloxy-2-hydroxy-propyl-oxy}propane, 1,2,3-tris{2-(meth)acryloyloxy-ethyl-oxy}propane, 1,2,3-tris{2-(meth)acryloyloxy-propyl-oxy}propane, 1,2,3-tris{(meth)acryloyloxy-poly(1,2-ethyleneoxy)}propane, 1,2,3-tris{(meth)acryloyloxy-poly(1,2-propyleneoxy)}propane, 1,2,3-tris{(meth)acryloyloxy-poly(1,3-propyleneoxy)}propane; trimethylolpropane tri(meth)acrylate, trimethylolpropane-tris{(meth)acryloyloxy-ethyl-oxy} ether, trimethylolpropane-tris{2-(meth)acryloyloxy-propyl-oxy} ether, trimethylolpropane-tris{(meth)acryloyloxy-poly(ethyleneoxy)} ether, trimethylolpropane-tris{(meth)acryloyloxy-poly(1,2-propyleneoxy)} ether, pentaerythritol tri(meth)acrylate, pentaerythritol tetra(meth)acrylate, pentaerythritol-tetrakis{(meth)acryloyloxy-ethyl-oxy} ether, pentaerythritol-tetrakis{2-(meth)acryloyloxy-propyl-oxy} ether, pentaerythritol-tetrakis{(meth)acryloyloxy-poly(ethyleneoxy)} ether, pentaerythritol-tetrakis{(meth)acryloyloxy-poly(1,2-propylene oxy)} ether; ditrimethylolpropane tetra(meth)acrylate, ditrimethylolpropane-tetrakis{(meth)acryloyloxy-ethyl-oxy} ether, ditrimethylolpropane-tetrakis{2-(meth)acryloyloxy-propyl-oxy} ether, ditrimethylolpropane-tetrakis{(meth)acryloyloxy-poly(ethylene oxy)} ether, ditrimethylolpropane-tetrakis{(meth)acryloyloxy-poly(1,2-propyleneoxy)} ether, dipentaerythritol penta(meth)acrylate, dipentaerythritol hexa(meth)acrylate, dipentaerythritol-hexa{(meth)acryloyloxy-ethyl-oxy} ether, dipentaerythritol-hexa{2-(meth)acryloyloxy-propyl-oxy} ether, dipentaerythritol-hexa{(meth)acryloyloxy-poly(ethyleneoxy)} ether, dipentaerythritol-hexa{(meth)acryloyloxy-poly(1,2-propyleneoxy)} ether; and so on.

In addition, examples of the compound (II) include a urethane reaction product of hexamethylene diisocyanate with 2-hydroxyethyl(meth)acrylate, 2-hydroxypropyl(meth)acrylate, 3-hydroxypropyl(meth)acrylate, or 4-hydroxybutyl(meth)acrylate; a urethane reaction product of isophorone diisocyanate with 2-hydroxyethyl(meth)acrylate, 2-hydroxypropyl(meth)acrylate, 3-hydroxypropyl(meth)acrylate, or 4-hydroxybutyl(meth)acrylate; a urethane reaction product of bis(isocyanatomethyl)norbornane with 2-hydroxyethyl(meth)acrylate, 2-hydroxypropyl(meth)acrylate, 3-hydroxypropyl(meth)acrylate, or 4-hydroxybutyl(meth)acrylate; a urethane reaction product of norbis(4-isocyanatocyclohexyl)methane with 2-hydroxyethyl(meth)acrylate, 2-hydroxypropyl(meth)acrylate, 3-hydroxypropyl(meth)acrylate, or 4-hydroxybutyl(meth)acrylate; a urethane reaction product of 1,3-bis(isocyanatomethyl)cyclohexane with 2-hydroxyethyl(meth)acrylate, 2-hydroxypropyl(meth)acrylate, 3-hydroxypropyl(meth)acrylate, or 4-hydroxybutyl(meth)acrylate; a urethane reaction product of m-xylylene diisocyanate with 2-hydroxyethyl(meth)acrylate, 2-hydroxypropyl(meth)acrylate, 3-hydroxypropyl(meth)acrylate, or 4-hydroxybutyl(meth)acrylate; and so on.

The compounds (II) may be used singly, or two or more may be used in combination. The compounds (II) may be produced by a known method or by a method in accordance with a known method, or may be purchased from the market.

The compound (I) and the compound (II) are preferably blended in such a ratio that the amount of the compound (I) is 0.1 to 50 wt % and the amount of the compound (II) is 99.9 to 50 wt % relative to the total weight of the compound (I) and the compound (II). More preferably, the amount of the compound (I) is 0.3 to 30 wt % and the amount of the compound (II) is 99.7 to 70 wt %. Still more preferably, the amount of the compound (I) is 0.5 to 20 wt % and the amount of the compound (II) is 99.5 to 80 wt %.

### <Surfactant (III)>

The dental composition of the invention contains the surfactant (III), in addition to the compound (I) and the compound (II). The surfactant (III) contained in the dental composition of the invention has a hydrophilic moiety including an anionic hydrophilic group, a cationic hydrophilic group or two or more hydroxyl groups, and a hydrophobic moiety composed of an organic residue, but has no polymerizable carbon-carbon double bond. The curing of the composition including such a surfactant (III) tends to result in an increased concentration of the hydrophilic groups derived from the compound (I) at the surface of the resulting cured product. When the cured product is a monolayer film, for example, the enrichment of the hydrophilic groups at the surface is facilitated.

Of the surfactants, those compounds represented by the general formula (300) below are preferable.

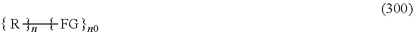

In the formula (300), R represents a C₄₋₁₀₀ organic residue; FG represents a hydrophilic group including at least one group selected from anionic hydrophilic groups, cationic hydrophilic groups and hydroxyl groups; n indicates the number of Rs bonded to FG and is 1 or 2; n0 indicates the number of FGs bonded to R and is an integer of 1 to 5; and when FG is a group including one hydroxyl group, n0 is an integer of 2 to 5.

As described above, FG contains at least one hydrophilic group selected from anionic hydrophilic groups, cationic hydrophilic groups and hydroxyl group.

Examples of the group FG containing an anionic hydrophilic group include hydrophilic groups represented by any of the general formulas (301) and (302).

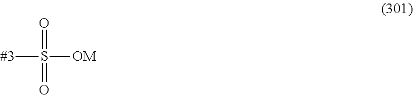

In the formula (301), M is a hydrogen atom, an alkali metal, an alkaline earth metal (½ atom) or an ammonium ion; and #3 indicates a hand bonded to a carbon atom present in R in the formula (300).

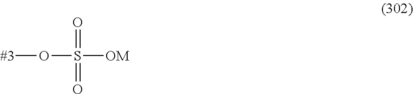

In the formula (302), M is a hydrogen atom, an alkali metal, an alkaline earth metal (½ atom) or an ammonium ion; and #3 indicates a hand bonded to a carbon atom present in R in the formula (300).

Examples of the surfactants in which FG is represented by the general formula (301) include alkylsulfonic acid surfactants, alkenylsulfonic acid surfactants (in which the alkenyl groups present in the surfactants are not polymerizable), alkyl acetic acid sulfonic acid surfactants, N-acylsulfonic acid surfactants, hydroxyalkanesulfonic acid surfactants, arylsulfonic acid surfactants and sulfosuccinic acid ester surfactants.

Examples of the alkylsulfonic acid surfactants include butylsulfonic acid, pentylsulfonic acid, hexylsulfonic acid, heptylsulfonic acid, octylsulfonic acid, nonylsulfonic acid, decylsulfonic acid, undecylsulfonic acid, dodecylsulfonic acid, tridecylsulfonic acid, tetradecylsulfonic acid, pentadecylsulfonic acid, hexadecylsulfonic acid, heptadecyl sulfonic acid, octadecylsulfonic acid, nonadecylsulfonic acid and icosanylsulfonic acid; and salts thereof such as sodium salts, potassium salts, ammonium salts, magnesium salts and calcium salts; and so on.

Examples of the alkenylsulfonic acid surfactants include butynylsulfonic acid, hexynylsulfonic acid, octynylsulfonic acid, decynylsulfonic acid, dodecynylsulfonic acid, tetradecynylsulfonic acid, hexadecynylsulfonic acid, octadecynylsulfonic acid, icosanylsulfonic acid, butynyloxysulfonic acid, hexynyloxysulfonic acid, octynyloxysulfonic acid, decynyloxysulfonic acid, dodecynyloxysulfonic acid, tetradecynyloxysulfonic acid, hexadecynyloxysulfonic acid, octadecynyloxysulfonic acid, icosanyloxysulfonic acid, butynyloxy-3-oxapentylsulfonic acid, hexynyloxy-3-oxapentylsulfonic acid, octynyloxy-3-oxapentylsulfonic acid, decynyloxy-3-oxapentylsulfonic acid, dodecynyloxy-3-oxapentylsulfonic acid, tetradecynyloxy-3-oxapentylsulfonic acid, hexadecynyloxy-3-oxapentylsulfonic acid, octadecynyloxy-3-oxapentylsulfonic acid, icosanyloxy-3-oxapentylsulfonic acid, butynyloxy-3,6-dioxaoctylsulfonic acid, hexynyloxy-3,6-dioxaoctylsulfonic acid, octynyloxy-3,6-dioxaoctylsulfonic acid, decynyloxy-3,6-dioxaoctylsulfonic acid, dodecynyloxy-3,6-dioxaoctylsulfonic acid, tetradecynyloxy-3,6-dioxaoctylsulfonic acid, hexadecynyloxy-3,6-dioxaoctylsulfonic acid, octadecynyloxy-3,6-dioxaoctylsulfonic acid, icosanyloxy-3,6-dioxaoctylsulfonic acid, butynyloxy-3,6,9-trioxaundecylsulfonic acid, hexynyloxy-3,6,9-trioxaundecylsulfonic acid, octynyloxy-3,6,9-trioxaundecylsulfonic acid, decynyloxy-3,6,9-trioxaundecylsulfonic acid, dodecynyloxy-3,6,9-trioxaundecylsulfonic acid, tetradecynyloxy-3,6,9-trioxaundecylsulfonic acid, hexadecynyloxy-3,6,9-trioxaundecylsulfonic acid, octadecynyloxy-3,6,9-trioxaundecylsulfonic acid and icosanyloxy-3,6,9-trioxaundecylsulfonic acid; and salts thereof such as sodium salts, potassium salts, ammonium salts, triethanolamine salts, magnesium salts and calcium salts; and so on.

Examples of the alkyl acetic acid sulfonic acid surfactants include ethyl α-sulfoacetate, propyl α-sulfoacetate, butyl α-sulfoacetate, pentyl α-sulfoacetate, hexyl α-sulfoacetate, heptyl α-sulfoacetate, octyl α-sulfoacetate, nonyl α-sulfoacetate, decyl α-sulfoacetate, dodecyl α-sulfoacetate, tetradecyl α-sulfoacetate, hexadecyl α-sulfoacetate, octadecyl α-sulfoacetate and icosyl α-sulfoacetate; and salts thereof such as sodium salts, potassium salts, ammonium salts, magnesium salts and calcium salts; and so on.

Examples of the N-acylsulfonic acid surfactants include 2-hexylic acid amido-ethanesulfonic acid, 2-octylic acid amido-ethanesulfonic acid, 2-lauric acid amido-ethanesulfonic acid, 2-myristic acid amido-ethanesulfonic acid, 2-palmitic acid amido-ethanesulfonic acid, 2-stearic acid amido-ethanesulfonic acid, 2-oleic acid amide-ethanesulfonic acid, 2-behenic acid amido-ethanesulfonic acid, N-methyl-2-hexylic acid amido-ethanesulfonic acid, N-methyl-2-octylic acid amido-ethanesulfonic acid, N-methyl-2-lauric acid amido-ethanesulfonic acid, N-methyl-2-myristic acid amido-ethanesulfonic acid, N-methyl-2-palmitic acid amido-ethanesulfonic acid, N-methyl-2-stearic acid amido-ethanesulfonic acid, N-methyl-2-oleic acid amido-ethanesulfonic acid, N-methyl-2-behenic acid amido-ethanesulfonic acid, 3-hexylic acid amido-propanesulfonic acid, 3-octylic acid amido-propanesulfonic acid, 3-lauric acid amido-propanesulfonic acid, 3-myristic acid amido-propanesulfonic acid, 3-palmitic acid amido-propanesulfonic acid, 3-stearic acid amido-propanesulfonic acid, 3-oleic acid amido-propanesulfonic acid and 3-behenic acid amido-propanesulfonic acid; and salts thereof such as sodium salts, potassium salts, ammonium salts, magnesium salts and calcium salts; and so on.

Examples of the hydroxyalkanesulfonic acid surfactants include 2-hydroxybutylsulfonic acid, 2-hydroxypentylsulfonic acid, 2-hydroxyhexylsulfonic acid, 2-hydroxyheptylsulfonic acid, 2-hydroxyoctylsulfonic acid, 2-hydroxynonylsulfonic acid, 2-hydroxydecylsulfonic acid, 2-hydroxyundecylsulfonic acid, 2-hydroxydodecylsulfonic acid, 2-hydroxytridecylsulfonic acid, 2-hydroxytetradecylsulfonic acid, 2-hydroxypentadecylsulfonic acid, 2-hydroxyhexadecylsulfonic acid, 2-hydroxyheptadecylsulfonic acid, 2-hydroxyoctadecylsulfonic acid, 2-hydroxynonadecylsulfonic acid, 2-hydroxyicosanylsulfonic acid, 3-hydroxybutylsulfonic acid, 3-hydroxypentylsulfonic acid, 3-hydroxyhexylsulfonic acid, 3-hydroxyheptylsulfonic acid, 3-hydroxyoctylsulfonic acid, 3-hydroxynonylsulfonic acid, 3-hydroxydecylsulfonic acid, 3-hydroxyundecylsulfonic acid, 3-hydroxydodecylsulfonic acid, 3-hydroxytridecylsulfonic acid, 3-hydroxytetradecylsulfonic acid, 3-hydroxypentadecylsulfonic acid, 3-hydroxyhexadecylsulfonic acid, 3-hydroxyheptadecylsulfonic acid, 3-hydroxyoctadecylsulfonic acid, 3-hydroxynonadecylsulfonic acid, 3-hydroxyicosanylsulfonic acid, 4-hydroxybutylsulfonic acid, 4-hydroxypentylsulfonic acid, 4-hydroxyhexylsulfonic acid, 4-hydroxyheptylsulfonic acid, 4-hydroxyoctylsulfonic acid, 4-hydroxynonylsulfonic acid, 4-hydroxydecylsulfonic acid, 4-hydroxyundecylsulfonic acid, 4-hydroxydodecylsulfonic acid, 4-hydroxytridecylsulfonic acid, 4-hydroxytetradecylsulfonic acid, 4-hydroxypentadecylsulfonic acid, 4-hydroxyhexadecylsulfonic acid, 4-hydroxyheptadecylsulfonic acid, 4-hydroxyoctadecylsulfonic acid, 4-hydroxynonadecylsulfonic acid and 4-hydroxyicosanylsulfonic acid; and salts thereof such as sodium salts, potassium salts, ammonium salts, magnesium salts and calcium salts; and so on.

Examples of the arylsulfonic acid surfactants include phenylsulfonic acid, methylbenzenesulfonic acid, ethylbenzenesulfonic acid, propylbenzenesulfonic acid, butylbenzenesulfonic acid, pentylbenzenesulfonic acid, hexylbenzenesulfonic acid, heptylbenzenesulfonic acid, octylbenzenesulfonic acid, nonylbenzenesulfonic acid, decylbenzenesulfonic acid, undecylbenzenesulfonic acid, dodecylbenzenesulfonic acid, tridecylbenzenesulfonic acid, tetradecylbenzenesulfonic acid, pentadecylbenzenesulfonic acid, hexadecylbenzenesulfonic acid, heptadecylbenzenesulfonic acid, octadecylbenzenesulfonic acid, nonadecylbenzenesulfonic acid, icosanylbenzenesulfonic acid, di(methyl)benzenesulfonic acid, di(ethyl)benzenesulfonic acid, di(propyl)benzenesulfonic acid, di(butyl)benzenesulfonic acid, di(pentyl)benzenesulfonic acid, di(hexyl)benzenesulfonic acid, di(heptyl)benzenesulfonic acid, di(octyl)benzenesulfonic acid, di(nonyl)benzenesulfonic acid, di(decyl)benzenesulfonic acid, di(undecyl)benzenesulfonic acid, di(dodecyl)benzenesulfonic acid, di(tridecyl)benzenesulfonic acid, di(tetradecyl)benzenesulfonic acid, di(pentadecyl)benzenesulfonic acid, di(hexadecyl)benzenesulfonic acid, di(heptadecyl)benzenesulfonic acid, di(octadecyl)benzenesulfonic acid, di(nonadecyl)benzenesulfonic acid, di(icosanyl)benzenesulfonic acid, tri(methyl)benzenesulfonic acid, tri(ethyl)benzenesulfonic acid, tri(propyl)benzenesulfonic acid, tri(butyl)benzenesulfonic acid, tri(pentyl)benzenesulfonic acid, tri(hexyl)benzenesulfonic acid, tri(heptyl)benzenesulfonic acid, tri(octyl)benzenesulfonic acid, tri(nonyl)benzenesulfonic acid, tri(decyl)benzenesulfonic acid, tri(undecyl)benzenesulfonic acid, tri(dodecyl)benzenesulfonic acid, tri(tridecyl)benzenesulfonic acid, tri(tetradecyl)benzenesulfonic acid, tri(pentadecyl)benzenesulfonic acid, tri(hexadecyl)benzenesulfonic acid, tri(heptadecyl)benzenesulfonic acid, tri(octadecyl)benzenesulfonic acid, tri(nonadecyl)benzenesulfonic acid, tri(icosanyl)benzenesulfonic acid, naphthalenesulfonic acid, methylnaphthalenesulfonic acid, ethylnaphthalenesulfonic acid, propylnaphthalenesulfonic acid, butylnaphthalenesulfonic acid, pentylnaphthalenesulfonic acid, hexylnaphthalenesulfonic acid, heptylnaphthalenesulfonic acid, octylnaphthalenesulfonic acid, nonylnaphthalenesulfonic acid, decylnaphthalenesulfonic acid, undecylnaphthalenesulfonic acid, dodecylnaphthalenesulfonic acid, tridecylnaphthalenesulfonic acid, tetradecylnaphthalenesulfonic acid, pentadecylnaphthalenesulfonic acid, hexadecylnaphthalenesulfonic acid, heptadecylnaphthalenesulfonic acid, octadecylnaphthalenesulfonic acid(stearylnaphthalenesulfonic acid), nonadecylnaphthalenesulfonic acid, icosanylnaphthalenesulfonic acid, di(methyl)naphthalenesulfonic acid, di(ethyl)naphthalenesulfonic acid, di(propyl)naphthalenesulfonic acid, di(butyl)naphthalenesulfonic acid, di(pentyl)naphthalenesulfonic acid, di(hexyl)naphthalenesulfonic acid, di(heptyl)naphthalenesulfonic acid, di(octyl)naphthalenesulfonic acid, di(nonyl)naphthalenesulfonic acid, di(decyl)naphthalenesulfonic acid, di(undecyl)naphthalenesulfonic acid, di(dodecyl)naphthalenesulfonic acid, di(tridecyl)naphthalenesulfonic acid, di(tetradecyl)naphthalenesulfonic acid, di(pentadecyl)naphthalenesulfonic acid, di(hexadecyl)naphthalenesulfonic acid, di(heptadecyl)naphthalenesulfonic acid, di(octadecyl)naphthalenesulfonic acid, di(nonadecyl)naphthalenesulfonic acid, di(icosanyl)naphthalenesulfonic acid, tri(methyl)naphthalenesulfonic acid, tri(ethyl)naphthalenesulfonic acid, tri(propyl)naphthalenesulfonic acid, tri(butyl)naphthalenesulfonic acid, tri(pentyl)naphthalenesulfonic acid, tri(hexyl)naphthalenesulfonic acid, tri(heptyl)naphthalenesulfonic acid, tri(octyl)naphthalenesulfonic acid, tri(nonyl)naphthalenesulfonic acid, tri(decyl)naphthalenesulfonic acid, tri(undecyl)naphthalenesulfonic acid, tri(dodecyl)naphthalenesulfonic acid, tri(tridecyl)naphthalenesulfonic acid, tri(tetradecyl)naphthalenesulfonic acid, tri(pentadecyl)naphthalenesulfonic acid, tri(hexadecyl)naphthalenesulfonic acid, tri(heptadecyl)naphthalenesulfonic acid, tri(octadecyl)naphthalenesulfonic acid, tri(nonadecyl)naphthalenesulfonic acid, tri(icosanyl)naphthalenesulfonic acid, naphthalenesulfonic acid formalin condensate, methylnaphthalenesulfonic acid formalin condensate, ethylnaphthalenesulfonic acid formalin condensate, propylnaphthalenesulfonic acid formalin condensate, butylnaphthalenesulfonic acid formalin condensate, pentylnaphthalenesulfonic acid formalin condensate, hexylnaphthalenesulfonic acid formalin condensate, heptylnaphthalenesulfonic acid formalin condensate, octylnaphthalenesulfonic acid formalin condensate, nonylnaphthalenesulfonic acid formalin condensate, decylnaphthalenesulfonic acid formalin condensate, undecylnaphthalenesulfonic acid formalin condensate, dodecylnaphthalenesulfonic acid formalin condensate, tridecylnaphthalenesulfonic acid formalin condensate, tetradecylnaphthalenesulfonic acid formalin condensate, pentadecylnaphthalenesulfonic acid formalin condensate, hexadecylnaphthalenesulfonic acid formalin condensate, heptadecylnaphthalenesulfonic acid formalin condensate, octadecylnaphthalenesulfonic acid (stearylnaphthalenesulfonic acid) formalin condensate, nonadecylnaphthalenesulfonic acid formalin condensate, icosanylnaphthalenesulfonic acid formalin condensate, di(methyl)naphthalenesulfonic acid formalin condensate, di(ethyl)naphthalenesulfonic acid formalin condensate, di(propyl)naphthalenesulfonic acid formalin condensate, di(butyl)naphthalenesulfonic acid formalin condensate, di(pentyl)naphthalenesulfonic acid formalin condensate, di(hexyl)naphthalenesulfonic acid formalin condensate, di(heptyl)naphthalenesulfonic acid formalin condensate, di(octyl)naphthalenesulfonic acid formalin condensate, di(nonyl)naphthalenesulfonic acid formalin condensate, di(decyl)naphthalenesulfonic acid formalin condensate, di(undecyl)naphthalenesulfonic acid formalin condensate, di(dodecyl)naphthalenesulfonic acid formalin condensate, di(tridecyl)naphthalenesulfonic acid formalin condensate, di(tetradecyl)naphthalenesulfonic acid formalin condensate, di(pentadecyl)naphthalenesulfonic acid formalin condensate, di(hexadecyl)naphthalenesulfonic acid formalin condensate, di(heptadecyl)naphthalenesulfonic acid formalin condensate, di(octadecyl)naphthalenesulfonic acid formalin condensate, di(nonadecyl)naphthalenesulfonic acid formalin condensate, di(icosanyl)naphthalenesulfonic acid formalin condensate, tri(methyl)naphthalenesulfonic acid formalin condensate, tri(ethyl)naphthalenesulfonic acid formalin condensate, tri(propyl)naphthalenesulfonic acid formalin condensate, tri(butyl)naphthalenesulfonic acid formalin condensate, tri(pentyl)naphthalenesulfonic acid formalin condensate, tri(hexyl)naphthalenesulfonic acid formalin condensate, tri(heptyl)naphthalenesulfonic acid formalin condensate, tri(octyl)naphthalenesulfonic acid formalin condensate, tri(nonyl)naphthalenesulfonic acid formalin condensate, tri(decyl)naphthalenesulfonic acid formalin condensate, tri(undecyl) naphthalenesulfonic acid formalin condensate, tri(dodecyl)naphthalenesulfonic acid formalin condensate, tri(tridecyl)naphthalenesulfonic acid formalin condensate, tri(tetradecyl) naphthalenesulfonic acid formalin condensate, tri(pentadecyl) naphthalenesulfonic acid formalin condensate, tri(hexadecyl)naphthalenesulfonic acid formalin condensate, tri(heptadecyl) naphthalenesulfonic acid formalin condensate, tri(octadecyl)naphthalenesulfonic acid formalin condensate, tri(nonadecyl)naphthalenesulfonic acid formalin condensate, tri(icosanyl)naphthalenesulfonic acid formalin condensate, diphenyl ether sulfonic acid, methyl diphenyl ether sulfonic acid, ethyl diphenyl ether sulfonic acid, propyl diphenyl ether sulfonic acid, butyl diphenyl ether sulfonic acid, pentyl diphenyl ether sulfonic acid, hexyl diphenyl ether sulfonic acid, heptyl diphenyl ether sulfonic acid, octyl diphenyl ether sulfonic acid, nonyl diphenyl ether sulfonic acid, decyl diphenyl ether sulfonic acid, undecyl diphenyl ether sulfonic acid, dodecyl diphenyl ether sulfonic acid, tridecyl diphenyl ether sulfonic acid, tetradecyl diphenyl ether sulfonic acid, pentadecyl diphenyl ether sulfonic acid, hexadecyl diphenyl ether sulfonic acid, heptadecyl diphenyl ether sulfonic acid, octadecyl diphenyl ether sulfonic acid, nonadecyl diphenyl ether sulfonic acid, icosanyl diphenyl ether sulfonic acid, diphenyl ether disulfonic acid, methyl diphenyl ether disulfonic acid, ethyl diphenyl ether disulfonic acid, propyl diphenyl ether disulfonic acid, butyl diphenyl ether disulfonic acid, pentyl diphenyl ether disulfonic acid, hexyl diphenyl ether disulfonic acid, heptyl diphenyl ether disulfonic acid, octyl diphenyl ether disulfonic acid, nonyl diphenyl ether disulfonic acid, decyl diphenyl ether disulfonic acid, undecyl diphenyl ether disulfonic acid, dodecyl diphenyl ether disulfonic acid, tridecyl diphenyl ether disulfonic acid, tetradecyl diphenyl ether disulfonic acid, pentadecyl diphenyl ether disulfonic acid, hexadecyl diphenyl ether disulfonic acid, heptadecyl diphenyl ether disulfonic acid, octadecyl diphenyl ether disulfonic acid, nonadecyl diphenyl ether disulfonic acid and icosanyl diphenyl ether disulfonic acid; and salts thereof such as sodium salts, potassium salts, ammonium salts, magnesium salts and calcium salts; and so on.

Examples of the sulfosuccinic acid ester surfactants include:

mono(methyl)sulfosuccinic acid ester, mono(ethyl) sulfosuccinic acid ester, mono(propyl) sulfosuccinic acid ester, mono(butyl)sulfosuccinic acid ester, mono(pentyl) sulfosuccinic acid ester, mono(hexyl) sulfosuccinic acid ester, mono(heptyl)sulfosuccinic acid ester, mono(octyl) sulfosuccinic acid ester, mono(nonyl) sulfosuccinic acid ester, mono(decyl)sulfosuccinic acid ester, mono(undecyl)sulfosuccinic acid ester, mono(dodecyl)sulfosuccinic acid ester, mono(tridecyl)sulfosuccinic acid ester, mono(tetradecyl) sulfosuccinic acid ester, mono(pentadecyl)sulfosuccinic acid ester, mono(hexadecyl)sulfosuccinic acid ester, mono(heptadecyl)sulfosuccinic acid ester, mono(octadecyl)sulfosuccinic acid ester, mono(nonadecyl)sulfosuccinic acid ester, mono(icosanyl)sulfosuccinic acid ester, mono(benzyl)sulfosuccinic acid ester, mono(butoxyethyl)sulfosuccinic acid ester, mono(hexyloxyethyl)sulfosuccinic acid ester, mono(octyloxyethyl) sulfosuccinic acid ester, mono(nonyloxyethyl)sulfosuccinic acid ester, mono(decyloxyethyl)sulfosuccinic acid ester, mono(undecyloxyethyl)sulfosuccinic acid ester, mono(dodecyloxyethyl)sulfosuccinic acid ester, mono(tridecyloxyethyl)sulfosuccinic acid ester, mono(tetradecyloxyethyl)sulfosuccinic acid ester, mono(pentadecyloxyethyl)sulfosuccinic acid ester, mono(hexadecyloxyethyl)sulfosuccinic acid ester, mono(heptadecyloxyethyl)sulfosuccinic acid ester, mono(octadecyloxyethyl)sulfosuccinic acid ester, mono(nonadecyloxyethyl)sulfosuccinic acid ester and mono(icosanyloxyethyl)sulfosuccinic acid ester; and salts thereof such as sodium salts, disodium salts, potassium salts, dipotassium salts, ammonium salts, diammonium salts, magnesium salts and calcium salts;

di(methyl)sulfosuccinic acid ester, di(ethyl) sulfosuccinic acid ester, di(propyl) sulfosuccinic acid ester, di(butyl)sulfosuccinic acid ester, di(pentyl) sulfosuccinic acid ester, di(hexyl) sulfosuccinic acid ester, di(heptyl)sulfosuccinic acid ester, di(octyl)sulfosuccinic acid ester, di(nonyl)sulfosuccinic acid ester, di(decyl)sulfosuccinic acid ester, di(undecyl)sulfosuccinic acid ester, di(dodecyl) sulfosuccinic acid ester, di(tridecyl)sulfosuccinic acid ester, di(tetradecyl)sulfosuccinic acid ester, di(pentadecyl)sulfosuccinic acid ester, di(hexadecyl)sulfosuccinic acid ester, di(heptadecyl)sulfosuccinic acid ester, di(octadecyl)sulfosuccinic acid ester, di(nonadecyl)sulfosuccinic acid ester, di(icosanyl)sulfosuccinic acid ester, dibenzylsulfosuccinic acid ester, di(butoxyethyl)sulfosuccinic acid ester, di(hexyloxyethyl)sulfosuccinic acid ester, di(octyloxyethyl)sulfosuccinic acid ester, di(nonyloxyethyl)sulfosuccinic acid ester, di(decyloxyethyl)sulfosuccinic acid ester, di(undecyloxyethyl)sulfosuccinic acid ester, di(dodecyloxyethyl)sulfosuccinic acid ester, di(tridecyloxyethyl)sulfosuccinic acid ester, di(tetradecyloxyethyl)sulfosuccinic acid ester, di(pentadecyloxyethyl)sulfosuccinic acid ester, di(hexadecyloxyethyl)sulfosuccinic acid ester and di(octadecyloxyethyl) sulfosuccinic acid ester; and salts thereof such as sodium salts, potassium salts, ammonium salts, magnesium salts and calcium salts;

(nonadecyloxyethyl)sulfosuccinic acid ester sodium and (icosanyloxyethyl)sulfosuccinic acid ester sodium; and so on.

Of the surfactants in which FG is represented by the general formula (301), those compounds in which the organic residue has 6 to 100 carbon atoms are preferable. Those compounds in which the organic residue has 8 to 60 carbon atoms are more preferable, and those compounds in which the organic residue has 10 to 40 carbon atoms are still more preferable. Of the surfactants described above, sulfosuccinic acid ester surfactants are relatively preferable.

Examples of the surfactants in which FG is represented by the general formula (302) include alcohol sulfuric acid ester salt surfactants, aryl sulfuric acid ester salt surfactants and alkenyl sulfuric acid salt surfactants (in which the alkenyl groups present in the surfactants are not polymerizable).

Examples of the alcohol sulfuric acid ester salt surfactants include butyl sulfuric acid ester, pentyl sulfuric acid ester, hexyl sulfuric acid ester, heptyl sulfuric acid ester, octyl sulfuric acid ester, nonyl sulfuric acid ester, decyl sulfuric acid ester, undecyl sulfuric acid ester, dodecyl sulfuric acid ester, tridecyl sulfuric acid ester, tetradecyl sulfuric acid ester, pentadecyl sulfuric acid ester, hexadecyl sulfuric acid ester, heptadecyl sulfuric acid ester, octadecyl sulfuric acid ester, nonadecyl sulfuric acid ester, icosanyl sulfuric acid ester, 3-lauric acid-2-hydroxy-propyl sulfuric acid ester, 3-myristic acid-2-hydroxy-propyl sulfuric acid ester, 3-palmitic acid-2-hydroxy-propyl sulfuric acid ester, 3-stearic acid-2-hydroxy-propyl sulfuric acid ester, 3-oleic acid-2-hydroxy-propyl sulfuric acid ester, 3-behenic acid-2-hydroxy-propyl sulfuric acid ester, ethylene glycol mono(octylphenyl) ether sulfuric acid ester, diethylene glycol mono(octylphenyl) ether sulfuric acid ester, triethylene glycol mono(octylphenyl) ether sulfuric acid ester,

tetraethylene glycol mono(octylphenyl) ether sulfuric acid ester, polyethylene glycol mono(octylphenyl) ether sulfuric acid ester, ethylene glycol mono(nonylphenyl) ether sulfuric acid ester, diethylene glycol mono(nonylphenyl) ether sulfuric acid ester, triethylene glycol mono(nonylphenyl) ether sulfuric acid ester, tetraethylene glycol mono(nonylphenyl) ether sulfuric acid ester, polyethylene glycol mono(nonylphenyl) ether sulfuric acid ester, butyloxyethyl sulfuric acid ester, isobutyloxyethyl sulfuric acid ester, t-butyloxyethyl sulfuric acid ester, pentyloxyethyl sulfuric acid ester, hexyloxyethyl sulfuric acid ester, heptyloxyethyl sulfuric acid ester, octyloxyethyl sulfuric acid ester, nonyloxyethyl sulfuric acid ester, decyloxyethyl sulfuric acid ester, undecyloxyethyl sulfuric acid ester, dodecyloxyethyl sulfuric acid ester (lauryloxyethyl sulfuric acid ester), tridecyloxyethyl sulfuric acid ester, tetradecyloxyethyl sulfuric acid ester, pentadecyloxyethyl sulfuric acid ester, hexadecyloxyethyl sulfuric acid ester, heptadecyloxyethyl sulfuric acid ester, octadecyloxyethyl sulfuric acid ester, nonadecyloxyethyl sulfuric acid ester, icosanyloxyethyl sulfuric acid ester, butyloxypropyl-2-sulfuric acid ester, isobutyloxypropyl-2-sulfuric acid ester, t-butyloxypropyl-2-sulfuric acid ester, pentyloxypropyl-2-sulfuric acid ester, hexyloxypropyl-2-sulfuric acid ester, heptyloxypropyl-2-sulfuric acid ester, octyloxypropyl-2-sulfuric acid ester, nonyloxypropyl-2-sulfuric acid ester, decyloxypropyl-2-sulfuric acid ester, undecyloxypropyl-2-sulfuric acid ester, dodecyloxypropyl-2-sulfuric acid ester (lauryloxypropyl-2-sulfuric acid ester), tridecyloxypropyl-2-sulfuric acid ester, tetradecyloxypropyl-2-sulfuric acid ester, pentadecyloxypropyl-2-sulfuric acid ester, hexadecyloxypropyl-2-sulfuric acid ester, heptadecyloxypropyl-2-sulfuric acid ester, octadecyloxypropyl-2-sulfuric acid ester, nonadecyloxypropyl-2-sulfuric acid ester, icosanyloxypropyl-2-sulfuric acid ester, butyloxy-3-oxapentyl sulfuric acid ester, isobutyloxy-3-oxapentyl sulfuric acid ester, t-butyloxy-3-oxapentyl sulfuric acid ester, pentyloxy-3-oxapentyl sulfuric acid ester, hexyloxy-3-oxapentyl sulfuric acid ester, heptyloxy-3-oxapentyl sulfuric acid ester, octyloxy-3-oxapentyl sulfuric acid ester, nonyloxy-3-oxapentyl sulfuric acid ester, decyloxy-3-oxapentyl sulfuric acid ester, undecyloxy-3-oxapentyl sulfuric acid ester, dodecyloxy-3-oxapentyl sulfuric acid ester (lauryloxy-3-oxapentyl sulfuric acid ester), tridecyloxy-3-oxapentyl sulfuric acid ester, tetradecyloxy-3-oxapentyl sulfuric acid ester, pentadecyloxy-3-oxapentyl sulfuric acid ester, hexadecyloxy-3-oxapentyl sulfuric acid ester, heptadecyloxy-3-oxapentyl sulfuric acid ester, octadecyloxy-3-oxapentyl sulfuric acid ester, nonadecyloxy-3-oxapentyl sulfuric acid ester, icosanyloxy-3-oxapentyl sulfuric acid ester, butyloxy-3,6-dioxaoctyl sulfuric acid ester, isobutyloxy-3,6-dioxaoctyl sulfuric acid ester, t-butyloxy-3,6-dioxaoctyl sulfuric acid ester, pentyloxy-3,6-dioxaoctyl sulfuric acid ester, hexyloxy-3,6-dioxaoctyl sulfuric acid ester, heptyloxy-3,6-dioxaoctyl sulfuric acid ester, octyloxy-3,6-dioxaoctyl sulfuric acid ester, nonyloxy-3,6-dioxaoctyl sulfuric acid ester, decyloxy-3,6-dioxaoctyl sulfuric acid ester, undecyloxy-3,6-dioxaoctyl sulfuric acid ester, dodecyloxy-3,6-dioxaoctyl sulfuric acid ester (lauryloxy-3,6-dioxaoctyl sulfuric acid ester), tridecyloxy-3,6-dioxaoctyl sulfuric acid ester, tetradecyloxy-3,6-dioxaoctyl sulfuric acid ester, pentadecyloxy-3,6-dioxaoctyl sulfuric acid ester, hexadecyloxy-3,6-dioxaoctyl sulfuric acid ester, heptadecyloxy-3,6-dioxaoctyl sulfuric acid ester, octadecyloxy-3,6-dioxaoctyl sulfuric acid ester, nonadecyloxy-3,6-dioxaoctyl sulfuric acid ester and icosanyloxy-3,6-dioxaoctyl sulfuric acid ester; and salts thereof such as triethanolamine salts, sodium salts, potassium salts, ammonium salts, magnesium salts and calcium salts; and so on.

Examples of the aryl sulfuric acid ester salt surfactants include phenyl sulfuric acid ester sodium, methyl benzene sulfuric acid ester sodium, ethyl benzene sulfuric acid ester sodium, propyl benzene sulfuric acid ester sodium, butyl benzene sulfuric acid ester sodium, pentyl benzene sulfuric acid ester sodium, hexyl benzene sulfuric acid ester sodium, heptyl benzene sulfuric acid ester sodium, octyl benzene sulfuric acid ester sodium, nonyl benzene sulfuric acid ester sodium, decyl benzene sulfuric acid ester sodium, undecyl benzene sulfuric acid ester sodium, dodecyl benzene sulfuric acid ester sodium, tridecyl benzene sulfuric acid ester sodium, tetradecyl benzene sulfuric acid ester sodium, pentadecyl benzene sulfuric acid ester sodium, hexadecyl benzene sulfuric acid ester sodium, heptadecyl benzene sulfuric acid ester sodium, octadecyl benzene sulfuric acid ester sodium, nonadecyl benzene sulfuric acid ester sodium, icosanyl benzene sulfuric acid ester sodium, 7-ethyl-2-methyl-undecane-4-sulfuric acid ester sodium, di(methyl)benzene sulfuric acid ester sodium, di(ethyl)benzene sulfuric acid ester sodium, di(propyl)benzene sulfuric acid ester sodium, di(butyl)benzene sulfuric acid ester sodium, di(pentyl)benzene sulfuric acid ester sodium, di(hexyl)benzene sulfuric acid ester sodium, di(heptyl)benzene sulfuric acid ester sodium, di(octyl)benzene sulfuric acid ester sodium, di(nonyl)benzene sulfuric acid ester sodium, di(decyl)benzene sulfuric acid ester sodium, di(undecyl)benzene sulfuric acid ester sodium, di(dodecyl)benzene sulfuric acid ester sodium, di(tridecyl)benzene sulfuric acid ester sodium, di(tetradecyl)benzene sulfuric acid ester sodium, di(pentadecyl)benzene sulfuric acid ester sodium, di(hexadecyl)benzene sulfuric acid ester sodium, di(heptadecyl)benzene sulfuric acid ester sodium, di(octadecyl)benzene sulfuric acid ester sodium, di(nonadecyl)benzene sulfuric acid ester sodium, di(icosanyl)benzene sulfuric acid ester sodium, tri(methyl)benzene sulfuric acid ester sodium, tri(ethyl)benzene sulfuric acid ester sodium, tri(propyl)benzene sulfuric acid ester sodium, tri(butyl)benzene sulfuric acid ester sodium, tri(pentyl)benzene sulfuric acid ester sodium, tri(hexyl)benzene sulfuric acid ester sodium, tri(heptyl)benzene sulfuric acid ester sodium, tri(octyl)benzene sulfuric acid ester sodium, tri(nonyl)benzene sulfuric acid ester sodium, tri(decyl)benzene sulfuric acid ester sodium, tri(undecyl)benzene sulfuric acid ester sodium, tri(dodecyl)benzene sulfuric acid ester sodium, tri(tridecyl)benzene sulfuric acid ester sodium, tri(tetradecyl)benzene sulfuric acid ester sodium, tri(pentadecyl)benzene sulfuric acid ester sodium, tri(hexadecyl)benzene sulfuric acid ester sodium, tri(heptadecyl)benzene sulfuric acid ester sodium, tri(octadecyl)benzene sulfuric acid ester sodium, tri(nonadecyl)benzene sulfuric acid ester sodium, tri(icosanyl)benzene sulfuric acid ester sodium, naphthalene sulfuric acid ester sodium, methyl naphthalene sulfuric acid ester sodium, ethyl naphthalene sulfuric acid ester sodium, propyl naphthalene sulfuric acid ester sodium, butyl naphthalene sulfuric acid ester sodium, pentyl naphthalene sulfuric acid ester sodium, hexyl naphthalene sulfuric acid ester sodium, heptyl naphthalene sulfuric acid ester sodium, octyl naphthalene sulfuric acid ester sodium, nonyl naphthalene sulfuric acid ester sodium, decyl naphthalene sulfuric acid ester sodium, undecyl naphthalene sulfuric acid ester sodium, dodecyl naphthalene sulfuric acid ester sodium, tridecyl naphthalene sulfuric acid ester sodium, tetradecyl naphthalene sulfuric acid ester sodium, pentadecyl naphthalene sulfuric acid ester sodium, hexadecyl naphthalene sulfuric acid ester sodium, heptadecyl naphthalene sulfuric acid ester sodium, octadecylnaphthalene sulfuric acid ester sodium, nonadecyl naphthalene sulfuric acid ester sodium, icosanyl naphthalene sulfuric acid ester sodium, di(methyl)naphthalene sulfuric acid ester sodium, di(ethyl)naphthalene sulfuric acid ester sodium, di(propyl)naphthalene sulfuric acid ester sodium, di(butyl)naphthalene sulfuric acid ester sodium, di(pentyl)naphthalene sulfuric acid ester sodium, di(hexyl)naphthalene sulfuric acid ester sodium, di(heptyl)naphthalene sulfuric acid ester sodium, di(octyl)naphthalene sulfuric acid ester sodium, di(nonyl)naphthalene sulfuric acid ester sodium, di(decyl)naphthalene sulfuric acid ester sodium, di(undecyl)naphthalene sulfuric acid ester sodium, di(dodecyl)naphthalene sulfuric acid ester sodium, di(tridecyl)naphthalene sulfuric acid ester sodium, di(tetradecyl)naphthalene sulfuric acid ester sodium, di(pentadecyl)naphthalene sulfuric acid ester sodium, di(hexadecyl)naphthalene sulfuric acid ester sodium, di(heptadecyl)naphthalene sulfuric acid ester sodium, di(octadecyl)naphthalene sulfuric acid ester sodium, di(nonadecyl)naphthalene sulfuric acid ester sodium, di(icosanyl)naphthalene sulfuric acid ester sodium, tri(methyl)naphthalene sulfuric acid ester sodium, tri(ethyl)naphthalene sulfuric acid ester sodium, tri(propyl)naphthalene sulfuric acid ester sodium, tri(butyl)naphthalene sulfuric acid ester sodium, tri(pentyl)naphthalene sulfuric acid ester sodium, tri(hexyl)naphthalene sulfuric acid ester sodium, tri(heptyl)naphthalene sulfuric acid ester sodium, tri(octyl)naphthalene sulfuric acid ester sodium, tri(nonyl)naphthalene sulfuric acid ester sodium, tri(decyl)naphthalene sulfuric acid ester sodium, tri(undecyl)naphthalene sulfuric acid ester sodium, tri(dodecyl)naphthalene sulfuric acid ester sodium, tri(tridecyl)naphthalene sulfuric acid ester sodium, tri(tetradecyl)naphthalene sulfuric acid ester sodium, tri(pentadecyl)naphthalene sulfuric acid ester sodium, tri(hexadecyl)naphthalene sulfuric acid ester sodium, tri(heptadecyl)naphthalene sulfuric acid ester sodium, tri(octadecyl)naphthalene sulfuric acid ester sodium, tri(nonadecyl)naphthalene sulfuric acid ester sodium, tri(icosanyl)naphthalene sulfuric acid ester sodium and so on.

Examples of the alkenyl sulfuric acid salt surfactants include butynyl sulfuric acid ester, hexynyl sulfuric acid ester, octynyl sulfuric acid ester, decynyl sulfuric acid ester, dodecynyl sulfuric acid ester, tetradecynyl sulfuric acid ester, hexadecynyl sulfuric acid ester, octadecynyl sulfuric acid ester, icosanyl sulfuric acid ester, butynyloxysulfuric acid ester, hexynyloxysulfuric acid ester, octynyloxysulfuric acid ester, decynyloxysulfuric acid ester, dodecynyloxysulfuric acid ester, tetradecynyloxysulfuric acid ester, hexadecynyloxysulfuric acid ester, octadecynyloxysulfuric acid ester, icosanyloxy sulfuric acid ester, butynyloxy-3-oxapentyl sulfuric acid ester, hexynyloxy-3-oxapentyl sulfuric acid ester, octynyloxy-3-oxapentyl sulfuric acid ester, decynyloxy-3-oxapentyl sulfuric acid ester, dodecynyloxy-3-oxapentyl sulfuric acid ester, tetradecynyloxy-3-oxapentyl sulfuric acid ester, hexadecynyloxy-3-oxapentyl sulfuric acid ester, octadecynyloxy-3-oxapentyl sulfuric acid ester, icosanyloxy-3-oxapentyl sulfuric acid ester, butynyloxy-3,6-dioxaoctyl sulfuric acid ester, hexynyloxy-3,6-dioxaoctyl sulfuric acid ester, octynyloxy-3,6-dioxaoctyl sulfuric acid ester, decynyloxy-3,6-dioxaoctyl sulfuric acid ester, dodecynyloxy-3,6-dioxaoctyl sulfuric acid ester, tetradecynyloxy-3,6-dioxaoctyl sulfuric acid ester, hexadecynyloxy-3,6-dioxaoctyl sulfuric acid ester, octadecynyloxy-3,6-dioxaoctyl sulfuric acid ester, icosanyloxy-3,6-dioxaoctyl sulfuric acid ester, butynyloxy-3,6,9-trioxaundecyl sulfuric acid ester, hexynyloxy-3,6,9-trioxaundecyl sulfuric acid ester, octynyloxy-3,6,9-trioxaundecyl sulfuric acid ester, decynyloxy-3,6,9-trioxaundecyl sulfuric acid ester, dodecynyloxy-3,6,9-trioxaundecyl sulfuric acid ester, tetradecynyloxy-3,6,9-trioxaundecyl sulfuric acid ester, hexadecynyloxy-3,6,9-trioxaundecyl sulfuric acid ester, octadecynyloxy-3,6,9-trioxaundecyl sulfuric acid ester and icosanyloxy-3,6,9-trioxaundecyl sulfuric acid ester; and salts thereof such as sodium salts, potassium salts, ammonium salts, triethanolamine salts, magnesium salts and calcium salts; and the like.

Of the surfactants in which FG is represented by the general formula (302), those compounds in which the organic residue has 6 to 100 carbon atoms are preferable. Those compounds in which the organic residue has 8 to 60 carbon atoms are more preferable, and those compounds in which the organic residue has 10 to 40 carbon atoms are still more preferable. Of the surfactants described above, alcohol sulfuric acid eater salt surfactants are relatively preferable.

Examples of the group FG containing a hydroxyl group include hydrophilic groups represented by the general formula (312) below.

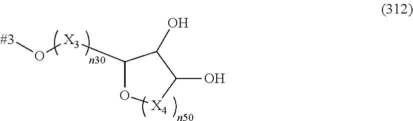

In the formula (312), X₃ and X₄ are each independently —CH₂—, —CH(OH)— or —CO—; n₃₀ is an integer of 0 to 3; n₅₀ is an integer of 0 to 5; when n₃₀ is 2 or greater, X₃s may be the same as or different from one another; when n₃₀ is 2 or greater, X₄s may be the same as or different from one another; and #3 indicates a hand bonded to a carbon atom present in R in the formula (300).

Examples of the surfactants wherein FG is represented by the general formula (312) include butyric acid ribose, valeric acid ribose, caproic acid ribose, caprylic acid ribose, capric acid ribose, lauric acid ribose, myristic acid ribose, palmitic acid ribose, stearic acid ribose, isostearic acid ribose, oleic acid ribose, behenic acid ribose, cyclohexanecarboxylic acid ribose, phenyl acetic acid ribose, butyric acid ascorbic acid, valeric acid ascorbic acid, caproic acid ascorbic acid, caprylic acid ascorbic acid, capric acid ascorbic acid, lauric acid ascorbic acid, myristic acid ascorbic acid, palmitic acid ascorbic acid, stearic acid ascorbic acid, isostearic acid ascorbic acid, oleic acid ascorbic acid, behenic acid ascorbic acid, cyclohexanecarboxylic acid ascorbic acid, phenyl acetic acid ascorbic acid, butyric acid xylol, valeric acid xylol, caproic acid xylol, caprylic acid xylol, capric acid xylol, lauric acid xylol, myristic acid xylol, palmitic acid xylol, stearic acid xylol, isostearic acid xylol, oleic acid xylol, behenic acid xylol, cyclohexanecarboxylic acid xylol, phenyl acetic acid xylol, butyric acid sorbitan, valeric acid sorbitan, caproic acid sorbitan, caprylic acid sorbitan, capric acid sorbitan, lauric acid sorbitan, myristic acid sorbitan, palmitic acid sorbitan, stearic acid sorbitan, isostearic acid sorbitan, oleic acid sorbitan, behenic acid sorbitan, cyclohexanecarboxylic acid sorbitan, phenyl acetic acid sorbitan, butyric acid glucose, valeric acid glucose, caproic acid glucose, caprylic acid glucose, capric acid glucose, lauric acid glucose, myristic acid glucose, palmitic acid glucose, stearic acid glucose, isostearic acid glucose, oleic acid glucose, behenic acid glucose, cyclohexanecarboxylic acid glucose, phenyl acetic acid glucose, butyric acid glucono-1,5-lactone, valeric acid glucono-1,5-lactone, caproic acid glucono-1,5-lactone, caprylic acid glucono-1,5-lactone, capric acid glucono-1,5-lactone, lauric acid glucono-1,5-lactone, myristic acid glucono-1, 5-lactone, palmitic acid glucono-1,5-lactone, stearic acid glucono-1,5-lactone, isostearic acid glucono-1,5-lactone, oleic acid glucono-1,5-lactone, behenic acid glucono-1, 5-lactone, cyclohexanecarboxylic acid glucono-1,5-lactone, phenyl acetic acid glucono-1,5-lactone, and adducts thereof such as ethylene oxide adduct, propylene oxide adduct, and butyrolactone adduct, and the dehydration-condensed multimer thereof and so on.

Of the surfactants represented by the general formula (312), those compounds in which the organic residue has 6 to 100 carbon atoms are preferable. Those compounds in which the organic residue has 8 to 60 carbon atoms are more preferable, and those compounds in which the organic residue has 10 to 40 carbon atoms are still more preferable.

Examples of the group FG containing a cationic hydrophilic group include hydrophilic groups represented by the general formula (318) below.

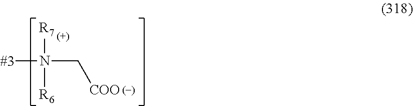

In the formula (318), R₆ and R₇ each independently represent a hydrogen atom, a C₁₋₂₀ alkyl, alkylaryl, alkylbenzyl, alkylcycloalkyl, alkylcycloalkylmethyl cycloalkyl group, a phenyl group or a benzyl group; and #3 indicates a hand bonded to R (a carbon atom present in R) in the formula (300).

Examples of the surfactants wherein FG is represented by the general formula (318) include butyl-dimethyl betaine, pentyl-dimethyl betaine, hexyl-dimethyl betaine, heptyl-dimethyl betaine, octyl-dimethyl betaine, nonyl-dimethyl betaine, decyl-dimethyl betaine, undecyl-dimethyl betaine, dodecyl-dimethyl betaine, tetradecyl-dimethyl betaine, tridecyl-dimethyl betaine, pentadecyl-dimethyl betaine, hexadecyl-dimethyl betaine, heptadecyl-dimethyl betaine, octadecyl-dimethyl betaine, nonadecyl-dimethyl betaine, icosanyl-dimethyl betaine, butyl-benzylmethyl betaine, pentyl-benzylmethyl betaine, hexyl-benzylmethyl betaine, heptyl-benzylmethyl betaine, octyl-benzylmethyl betaine, nonyl-benzylmethyl betaine, decyl-benzylmethyl betaine, undecyl-benzylmethyl betaine, dodecyl-benzylmethyl betaine, tridecyl benzylmethyl betaine, tetradecyl benzylmethyl betaine, pentadecyl-benzylmethyl betaine, hexadecyl-benzylmethyl betaine, heptadecyl-benzylmethyl betaine, octadecyl-benzylmethyl betaine, nonadecyl-benzylmethyl betaine, icosanyl-benzylmethyl betaine, butyl-cyclohexyl methyl betaine, pentyl-cyclohexyl methyl betaine, hexyl-cyclohexyl methyl betaine, heptyl-cyclohexyl methyl betaine, octyl-cyclohexyl methyl betaine, nonyl-cyclohexyl methyl betaine, decyl-cyclohexyl methyl betaine, undecyl-cyclohexyl methyl betaine, dodecyl-cyclohexyl methyl betaine, tridecyl cyclohexyl methyl betaine, tetradecyl cyclohexyl methyl betaine, pentadecyl-cyclohexyl methyl betaine, hexadecyl-cyclohexyl methyl betaine, heptadecyl-cyclohexyl methyl betaine, octadecyl-cyclohexyl methyl betaine, nonadecyl-cyclohexyl methyl betaine, icosanyl-cyclohexyl methyl betaine, butyl-dodecyl methyl betaine, pentyl-dodecyl methyl betaine, hexyl-dodecyl methyl betaine, heptyl-dodecyl methyl betaine, octyl-dodecyl methyl betaine, nonyl-dodecyl methyl betaine, decyl-dodecyl methyl betaine, undecyl-dodecyl methyl betaine, dodecyl-dodecyl methyl betaine, tridecyl dodecyl methyl betaine, tetradecyl dodecyl methyl betaine, pentadecyl-dodecyl methyl betaine, hexadecyl-dodecyl methyl betaine, heptadecyl-dodecyl methyl betaine, octadecyl-dodecyl methyl betaine, nonadecyl-dodecyl methyl betaine, icosanyl-dodecyl methyl betaine, and adducts thereof such as hydrogen halide adduct, carboxylic acid adduct, ammonia adduct, amine adduct, alkali metal hydroxide adduct, and alkaline-earth metal hydroxide adduct and so on.

Of the surfactants in which FG is represented by the general formula (318), those compounds in which the organic residue has 6 to 100 carbon atoms are preferable. Those compounds in which the organic residue has 8 to 60 carbon atoms are more preferable, and those compounds in which the organic residue has 10 to 40 carbon atoms are still more preferable.

The composition of the invention contains the surfactant (III) usually in the range of 0.0001 to 50 wt %, preferably in the range of 0.001 to 20 wt %, and more preferably in the range of 0.01 to 10 wt %, with respect to the total weight of the compound (I) and the compound (II). The curing of the composition containing the surfactant (III) in the above mentioned range tends to result in an increased concentration of the hydrophilic groups derived from the compound (I) at the surface of the resulting cured product. When the cured product is a monolayer film, for example, the enrichment of the hydrophilic groups at the surface is facilitated.

### <Other Components>

The dental composition of the invention may further contain other components as required.

Examples of the other components include polymerization initiators, polymerization accelerators, UV absorbers, hindered amine light stabilizers (HALS), solvents, fillers, antioxidants, polymerization inhibitors, pigments, antibacterial agents, X-ray contrast media, thickeners, fluorescent agents, and so on.

### Polymerization Initiators

When a dental cured product (hydrophilic dental cured product) of the invention to be described later is produced from the dental composition of the invention, the dental composition is cured to be formed, for example, in the form of a monolayer film. In the production of the cured product, any polymerization initiator generally used in the field of dentistry can be used. The polymerization initiator is usually selected in view of the polymerizability of the polymerizable monomer and the polymerization conditions used.

When the curing of the dental composition of the invention is carried out at a normal temperature, a redox polymerization initiator, which is a combination of an oxidizing agent and a reducing agent, is preferably used for example. In the case of using the redox polymerization initiator, it is necessary that the oxidizing agent and the reducing agent be packaged separately, so that they can be mixed immediately before use.

Examples of the oxidizing agent include organic peroxides such as diacyl peroxides, peroxy esters, dialkyl peroxides, peroxyketals, ketone peroxides and hydroperoxides, but not particularly limited thereto. Examples of the organic peroxides include: diacyl peroxides such as benzoyl peroxide, 2,4-dichlorobenzoyl peroxide and m-toluoyl peroxide; peroxy esters such as t-butyl peroxybenzoate, bis-t-butyl peroxyisophthalate, 2,5-dimethyl-2,5-bis(benzoylperoxy)hexane, t-butyl peroxy-2-ethylhexanoate and t-butyl peroxyisopropylcarbonate; dialkyl peroxides such as dicumyl peroxide, di-t-butyl peroxide and lauroyl peroxide; peroxyketals such as 1,1-bis(t-butylperoxy)-3,3,5-trimethylcyclohexane; ketone peroxides such as methyl ethyl ketoneperoxide; hydroperoxides such as t-butyl hydroperoxide; and so on.

The reducing agent is not particularly limited, but a tertiary amine is usually used. Examples of the tertiary amine include N,N-dimethylaniline, N,N-dimethyl-p-toluidine, N,N-dimethyl-m-toluidine, N,N-diethyl-p-toluidine, N,N-dimethyl-3,5-dimethylaniline, N,N-dimethyl-3,4-dimethylaniline, N,N-dimethyl-4-ethylaniline, N,N-dimethyl-4-i-propylaniline, N,N-dimethyl-4-t-butylaniline, N,N-dimethyl-3,5-di-t-butylaniline, N,N-bis(2-hydroxyethyl)-p-toluidine, N,N-bis(2-hydroxyethyl)-3,5-dimethylaniline, N,N-bis(2-hydroxyethyl)-3,4-dimethylaniline, N,N-bis(2-hydroxyethyl)-4-ethylaniline, N,N-bis(2-hydroxyethyl)-4-i-propylaniline, N,N-bis(2-hydroxyethyl)-4-t-butylaniline, N,N-di(2-hydroxyethyl)-3,5-di-i-propylaniline, N,N-bis(2-hydroxyethyl)-3,5-di-t-butylaniline, ethyl 4-dimethylaminobenzoate, n-butoxyethyl 4-dimethylaminobenzoate, (2-methacryloyloxy)ethyl 4-dimethylaminobenzoate, trimethylamine, triethylamine, N-methyldiethanolamine, N-ethyldiethanolamine, N-n-butyldiethanolamine, N-lauryldiethanolamine, triethanolamine, (2-dimethylamino)ethylmethacrylate, N,N-bis(methacryloyloxyethyl)-N-methylamine, N,N-bis(methacryloyloxyethyl)-N-ethylamine, N,N-bis(2-hydroxyethyl)-N-methacryloyloxyethylamine, N,N-bis(methacryloyloxyethyl)-N-(2-hydroxyethyl)amine, tris(methacryloyloxyethyl)amine, and so on.

In addition to the organic peroxide/amine-based redox systems as described above, cumene hydroperoxide/thiourea-based systems, ascorbic acid/Cu²⁺-based systems, and organic peroxide/amine/sulfinic acid (or salts thereof)-based systems can be used as the redox polymerization initiator. Further, tributylborane, an organic sulfinic acid and the like can also be used as the polymerization initiator.

When the dental composition of the invention is cured by radiation, such as UV light, a photopolymerization initiator is added to the mixture. When the dental composition is cured by heat, a thermal polymerization initiator is added.

Examples of the photopolymerization initiators include photo radical polymerization initiators, photo cationic polymerization initiators and photo anionic polymerization initiators. Of these photopolymerization initiators, photo radical polymerization initiators are preferable.

Examples of the photo radical polymerization initiators include IRGACURE 127 (manufactured by Ciba Specialty Chemicals Inc.), IRGACURE 651 (manufactured by Ciba Specialty Chemicals Inc.), IRGACURE 184 (manufactured by Ciba Specialty Chemicals Inc.), DAROCUR 1173 (manufactured by Ciba Specialty Chemicals Inc.), benzophenone, 4-phenylbenzophenone, IRGACURE 500 (manufactured by Ciba Specialty Chemicals Inc.), IRGACURE 2959 (manufactured by Ciba Specialty Chemicals Inc.), IRGACURE 907 (manufactured by Ciba Specialty Chemicals Inc.), IRGACURE 369 (manufactured by Ciba Specialty Chemicals Inc.), IRGACURE 1300 (manufactured by Ciba Specialty Chemicals Inc.), IRGACURE 819 (manufactured by Ciba Specialty Chemicals Inc.), Speedcure CPTX (manufactured by LAMBSON Ltd.), Speedcure DETX (manufactured by LAMBSON Ltd.), Speedcure CTX (manufactured by LABSON Ltd.), Speedcure ITX (manufactured by LAMBSON Ltd.), IRGACURE 379EG (manufactured by Ciba Specialty Chemicals Inc.), IRGACURE 1800 (manufactured by Ciba Specialty Chemicals Inc.), DAROCUR TPO (manufactured by Ciba Specialty Chemicals Inc.; (2,4,6-trimethylbenzoyl)diphenylphosphine oxide), DAROCUR 4265 (manufactured by Ciba Specialty Chemicals Inc.), IRGACURE OXE01 (manufactured by Ciba Specialty Chemicals Inc.), IRGACURE OXE02 (manufactured by Ciba Specialty Chemicals Inc.)), ESACURE KT55 (manufactured by Lamberti S.P.A.), ESACURE ONE (manufactured by Lamberti S.P.A.), ESACURE KIP150 (manufactured by Lamberti S.P.A.), ESACURE KIP100F (manufactured by Lamberti S.P.A.), ESACURE KT37 (manufactured by Lamberti S.P.A.), ESACURE KTO46 (manufactured by Lamberti S.P.A.), ESACURE 1001M (manufactured by Lamberti S.P.A.), ESACURE KIP/EM (manufactured by Lamberti S.P.A.), ESACURE DP250 (manufactured by Lamberti S.P.A.), ESACURE KB1 (manufactured by Lamberti S.P.A.), camphorquinone, 2-ethylanthraquinone, N,N-dimethyl-p-toluidine, benzil, 2,3-pentanedione, benzyl dimethyl ketal, benzyl diethyl ketal, 2-chlorothioxanthone, 2,4-diethylthioxanthone, 2,4, 6-trimethylbenzoyldiphenylphosphine oxide, 2,6-dimethoxybenzoyldiphenylphosphine oxide, 2,6-dichlorobenzoyldiphenylphosphine oxide, 2,3,5,6-tetramethylbenzoyldiphenylphosphine oxide, benzoylbis(2,6-dimethylphenyl) phosphonate, 2,4,6-trimethylbenzoylethoxyphenylphosphine oxide, 3,3′-carbonylbis(7-diethylamino)cumarin, 3-(4-methoxybenzoyl)cumarin, 3-thienoylcumarin, 2,4,6-tris(trichloromethyl)-s-triazine, 2,4,6-tris(tribromomethyl)-s-triazine, 2-methyl-4,6-bis(trichloromethyl)-s-triazine, and so on.

Of the above mentioned photopolymerization initiators, for example, IRGACURE 127 (manufactured by Ciba Specialty Chemicals Inc.), IRGACURE 184 (manufactured by Ciba Specialty Chemicals Inc.), DAROCUR 1173 (manufactured by Ciba Specialty Chemicals Inc.), IRGACURE 500 (manufactured by Ciba Specialty Chemicals Inc.), IRGACURE 819 (manufactured by Ciba Specialty Chemicals Inc.), DAROCUR TPO (manufactured by Ciba Specialty Chemicals Inc.), ESACURE ONE (manufactured by Lamberti S.P.A.), ESACURE KIP100F (manufactured by Lamberti S.P.A.), ESACURE KT37 (manufactured by Lamberti S.P.A.), ESACURE KTO46 (manufactured by Lamberti S.P.A.), and camphorquinone are preferred.

Examples of the photo cationic polymerization initiators include IRGACURE 250 (manufactured by Ciba Specialty Chemicals Inc.), IRGACURE 784 (manufactured by Ciba Specialty Chemicals Inc.), ESACURE 1064 (manufactured by Lamberti S.P.A.), CYRAURE UVI6990 (manufactured by Union Carbide Corp. Japan), ADEKA OPTOMER SP-172 (manufactured by ADEKA CORPORATION), ADEKA OPTOMER SP-170 (manufactured by ADEKA CORPORATION), ADEKA OPTOMER SP-152 (manufactured by ADEKA CORPORATION) and ADEKA OPTOMER SP-150 (manufactured by ADEKA CORPORATION).

In the case of using the photopolymerization initiator, a reducing agent can be used in combination in order to facilitate the photocurability.

Primary examples of the reducing agent include tertiary amines, aldehydes and compounds containing a thiol group. These may be used singly, or in combination of two or more.

Examples of the tertiary amines include 2-dimethylaminoethyl(meth)acrylate, N,N-bis[(meth)acryloyloxyethyl]-N-methylamine, ethyl 4-dimethylaminobenzoate, butyl 4-dimethylaminobenzoate, butoxyethyl 4-dimethylaminobenzoate, N-methyldiethanolamine, 4-dimethylaminobenzophenone, and so on.

Examples of the aldehydes include dimethylaminobenzaldehyde, terephthalaldehyde, and so on.

Examples of the compounds containing a thiol group include 2-mercaptobenzooxazole, decanethiol, 3-mercaptopropyltrimethoxysilane, thiobenzoic acid, and so on.

Further, in the case of using the photopolymerization initiator, a photopolymerization accelerator may be used in combination. Examples of the photopolymerization accelerator include 2,2-bis(2-chlorophenyl)-4,5′-tetraphenyl-2′H-<1,2′>biimidazol yl, tris(4-dimethylaminophenyl)methane, 4,4′-bis(dimethylamino)benzophenone, 2-ethylanthraquinone and camphorquinone.

Examples of the thermal polymerization initiator include: ketone peroxides such as methyl isobutyl ketone peroxide and cyclohexanone peroxide;

diacyl peroxides such as isobutyryl peroxide, o-chlorobenzoyl peroxide and benzoyl peroxide;

dialkyl peroxides such as tris(t-butylperoxy)triazine and t-butylcumyl peroxide;

peroxyketals such as 2,2-bis(4,4-di-t-butylperoxycyclohexyl)propane and 2,2-di(t-butylperoxy)butane;

alkyl peresters such as α-cumyl peroxyneodecanoate, t-butyl peroxypivalate, 2,4,4-trimethylpentyl peroxy-2-ethylhexanoate, t-butyl peroxy-2-ethylhexanoate and t-butyl peroxy-3,5,5-trimethylhexanoate; and

percarbonates such as di-3-methoxybutyl peroxydicarbonate, bis(4-t-butylcyclohexyl)peroxydicarbonate, t-butyl peroxyisopropyl carbonate and diethylene glycol bis(t-butyl peroxycarbonate).

The photopolymerization initiator and the thermal polymerization initiator may each be used singly, or in combination of two or more.

The amount of the photopolymerization initiator(s) and the thermal polymerization initiator(s) used is preferably in the range of 0.01 to 20 wt %, more preferably in the range of 0.05 to 10 wt %, and still more preferably in the range of 0.1 to 5 wt %, with respect to the total amount of the compound (I) and the compound (II).

### UV Absorbers and Hindered Amine Light Stabilizers

To ensure that the hydrophilic dental cured product according to the invention, such as a dental monolayer film, can be used as an antifouling material, for example, without denaturing even under prolonged exposure to the exterior environment, it is desirable to impart weather resistance to the composition of the present invention by further incorporating an UV absorber and/or a hindered amine light stabilizer. The same applies for the production of a dental prosthesis including the above mentioned monolayer film.

The UV absorbers are not particularly limited. Various UV absorbers may be used, with examples including benzotriazole UV absorbers, triazine UV absorbers, benzophenone UV absorbers, benzoate UV absorbers, propanedioate ester UV absorbers and oxanilide UV absorbers.

Examples of the UV absorbers include benzotriazole UV absorbers such as 2-(2H-benzotriazol-2-yl)-p-cresol, 2-(2H-benzotriazol-2-yl)-4-tert-butylphenol, 2-(2H-benzotriazol-2-yl)-4,6-di-tert-butylphenol, 2-(2H-benzotriazol-2-yl)-4,6-bis(1-methyl-1-phenylethyl)phenol, 2-(2H-benzotriazol-2-yl)-4-(1,1,3,3-tetramethylbutyl)-6-(1-methyl-1-phenylethyl)phenol, 2-(2H-benzotriazol-2-yl)-4-(3-on-4-oxa-dodecyl)-6-tert-butyl-phenol, 2-(5-chloro(2H)-benzotriazol-2-yl)-4-(3-on-4-oxa-dodecyl)-6-tert-butyl-phenol, 2-(5-chloro(2H)-benzotriazol-2-yl)-4-methyl-6-tert-butyl-phenol, 2-(2H-benzotriazol-2-yl)-4,6-di-tert-pentylphenol, 2-(5-chloro(2H)-benzotriazol-2-yl)-4,6-di-tert-butylphenol, 2-(2H-benzotriazol-2-yl)-4-tert-octylphenol, 2-(2H-benzotriazol-2-yl)-4-methyl-6-n-dodecylphenol, and methyl-3-{3-(2H-benzotriazol-2-yl)-5-tert-butyl-4-hydroxyphenyl} propionate/polyethylene glycol 300 reaction product; triazine UV absorbers such as 2-(4-phenoxy-2-hydroxy-phenyl)-4,6-diphenyl-1,3,5-triazine, 2-(2-hydroxy-4-oxa-hexadecyloxy)-4,6-di(2, 4-dimethyl-phenyl)-1,3,5-triazine, 2-(2-hydroxy-4-oxa-heptadecyloxy)-4,6-di(2,4-dimethyl-phenyl)-1,3,5-triazine, 2-(2-hydroxy-4-iso-octyloxy-phenyl)-4,6-di(2,4-dimethyl-phenyl)-1,3,5-triazine, TINUVIN 400 (trade name, manufactured by Ciba Specialty Chemicals, Inc.), TINUVIN 405 (trade name, manufactured by Ciba Specialty Chemicals, Inc.), TINUVIN 460 (trade name, manufactured by Ciba Specialty Chemicals, Inc.) and TINUVIN 479 (trade name, manufactured by Ciba Specialty Chemicals, Inc.); benzophenone UV absorbers such as 2-hydroxy-4-n-octoxybenzophenone; benzoate UV absorbers such as 2,4-di-tert-butylphenyl-3,5-di-tert-butyl-4-hydroxybenzoate; propanedioate ester UV absorbers such as {(4-methoxyphenyl)-methylene}-dimethyl propanedioate ester, HOSTAVIN PR-25 (trade name, manufactured by Clariant (Japan) K.K.) and HOSTAVIN B-CAP (trade name, manufactured by Clariant (Japan) K.K.); and oxanilide UV absorbers such as 2-ethyl-2′-ethoxy-oxanilide and Sanduvor VSU (trade name, manufactured by Clariant (Japan) K.K.). Of these UV absorbers, triazine UV absorbers tend to be preferred.

The term hindered amine light stabilizers (abbreviated as HALs) is used to collectively refer to compounds which usually have a 2,2,6,6-tetramethylpiperidine skeleton. HALs are broadly categorized into low-molecular weight HALs, medium-molecular weight HALs, high-molecular weight HALs and reactive HALs, based on the molecular weight. Examples of the hindered amine light stabilizers include TINUVIN 111FDL (trade name, (manufactured by Ciba Specialty Chemicals Inc.), bis(1-octyloxy-2,2,6,6-tetramethyl-4-piperidyl) sebacate (TINUVIN 123 (trade name, manufactured by Ciba Specialty Chemicals Inc.)), TINUVIN 144 (trade name, manufactured by Ciba Specialty Chemicals Inc.), TINUVIN 292 (trade name, manufactured by Ciba Specialty Chemicals Inc.), TINUVIN 765 (trade name, manufactured by Ciba Specialty Chemicals Inc.), TINUVIN 770 (trade name, manufactured by Ciba Specialty Chemicals Inc.), N,N′-bis(3-aminopropyl)ethylenediamine-2,4-bis[N-butyl-N-(1,2,2,6,6-pentamethyl-4-piperidyl)amino]-6-chloro-1,3,5-triazine condensate (CHIMASSORB 119FL (trade name, manufactured by Ciba Specialty Chemicals Inc.)), CHIMASSORB 2020FDL (trade name, manufactured by Ciba Specialty Chemicals Inc.), dimethyl succinate-1-(2-hydroxyethyl)-4-hydroxy-2,2,6,6-tetramethylpiperidine polycondensate (CHIMASSORB 622LD (trade name, manufactured by Ciba Specialty Chemicals Inc.)), poly[{6-(1,1,3,3-tetramethyl-butyl)amino-1,3,5-triazine-2,4-diyl}{(2,2,6,6-tetramethyl-4-piperidyl)imino}hexamethylene{(2, 2,6,6-tetramethyllauryl-4-piperidyl)imino}] (CHIMASSORB 944FD (trade name, manufactured by Ciba Specialty Chemicals Inc.)), Sanduvor 3050 Liq. (trade name, manufactured by Clariant (Japan) K.K.), Sanduvor 3052 Liq. (trade name, (manufactured by Clariant (Japan) K.K.), Sanduvor 3058 Liq. (trade name, manufactured by Clariant (Japan) K.K.), Sanduvor 3051 Powder. (trade name, manufactured by Clariant (Japan) K.K.), Sanduvor 3070 Powder. (trade name, manufactured by Clariant (Japan) K.K.), VP Sanduvor PR-31 (trade name, manufactured by Clariant (Japan) K.K.), HOSTAVIN N20 (trade name, manufactured by Clariant (Japan) K.K.), HOSTAVIN N24 (trade name, manufactured by Clariant (Japan) K.K.), HOSTAVIN N30 (trade name, manufactured by Clariant (Japan) K.K.), HOSTAVIN N321 (trade name, manufactured by Clariant (Japan) K.K.), HOSTAVIN PR-31 (trade name, manufactured by Clariant (Japan) K.K.), HOSTAVIN 845 (trade name, manufactured by Clariant (Japan) K.K.) and NYLOSTAB S-EED (trade name, manufactured by Clariant (Japan) K.K.).

The UV absorber and the hindered amine light stabilizer may be added in any amounts without limitation. The amount of the UV absorber is usually 0.1 to 20 wt %, and preferably 0.5 to 10 wt %; and the amount of the hindered amine light stabilizer is usually 0.1 to 10 wt %, preferably 0.5 to 5 wt %, and more preferably 1 to 3 wt % relative to the total of the compounds (I) and (II). When the added amounts of the UV absorber and the hindered amine light stabilizer are in the above ranges, a cured product, such as a monolayer film, obtained from the composition of the invention, will have a markedly improved weather resistance. The addition of the UV absorber and the hindered amine light stabilizer in amounts less than the above ranges tends to result in a decreased improvement in the weather resistance of the resulting cured product, such as a monolayer film. On the other hand, the addition of the UV absorber and the hindered amine light stabilizer in amounts exceeding the above ranges may result in insufficient copolymerization reaction of the compound (I) with the compound (II), when the dental composition of the invention is cured.

### Solvents

Since the composition of the invention contains the surfactant (III) in addition to the compound (I) and the compound (II), it is possible to obtain a cured product enriched with hydrophilic groups at the surface, even in the absence of a solvent. However, the composition of the invention may include a solvent, in view of aspects such as the workability in the production of the cured product, for example, a monolayer film, from the composition.

The solvent is not particularly limited as long as the composition is capable of producing a cured product having a hydrophilic surface. However, it is not preferred to use a solvent which interacts excessively with a constituent included in the monomer composition used in the invention, for example, a solvent which reacts or forms a salt with the constituent, or a solvent having an excessively high boiling point, such as one having a boiling point exceeding 200° C. For example, ethanolamine compounds having a hydroxyethylamino structure [NRaRb(CH₂CH₂OH): wherein Ra and Rb are each independently hydrogen, a C₁₋₁₅ alkyl group or a CH₂CH₂OH group], such as ethanolamine, diethanolamine, triethanolamine, N-ethyl-ethanolamine, N-(2-ethylhexyl)ethanolamine, N-butyl-diethanolamine, N-hexyl-diethanolamine, N-lauryl-diethanolamine and N-cetyl-diethanolamine, are prone to interact with the hydrophilic groups present in the compound (I), for example, the anionic hydrophilic groups such as sulfo group, to form a salt or a pseudo salt, and are difficult to evaporate. Therefore, an attempt to remove such a solvent from the coated mixture may fail, since the solvent does not easily move to the surface in contact with the air, and tends to remain inside the coating. Consequently, the hydrophilic groups present in the compound (I) tend to be prevented from being enriched (concentrated) at the surface of the coating in contact with the air. Thus, such an ethanolamine compound is not suitably used as the solvent.

Any appropriate solvent, except for those described above, may be used, in view of aspects such as the solubility of the compound (I), the compound (II) and the surfactant (III).

For example, in a conventional composition, a solvent having a relatively higher polarity, such as a solvent having a solubility parameter (SP value) σ of 9.3 (cal/cm³)^{1/2} or more has been preferably used. However, the composition of the invention is capable of producing a cured product enriched with hydrophilic groups at the surface, even with the use of a solvent having a SP value of less than 9.3.

Note, however, that in cases where the composition of the invention is used in a state where a relatively large amount of solvent is contained (low solids content), the use of a large amount of a low-polarity solvent (s) alone may cause the separation of the compound (I) or the compound (II), resulting in a failure to produce a composition having a uniform composition. If the composition in such a state is coated on a substrate, it may result in a failure to obtain a coating (such as a coating film) having a uniform composition. Therefore, in terms of solubility, the composition of the invention including at least one high-polarity solvent tends to be preferred. The high-polarity solvent is preferably a solvent having a solubility parameter (SP value) a of 9.0 (cal/cm³)^{1/2} or more.

Examples of the solvent having a SP value in the preferred range include methanol, ethanol, 1-propanol, isopropanol (IPA), 1-butanol, isobutanol, 1-pentanol (1-amyl alcohol), isopentanol, 2-pentanol, 3-pentanol, cyclohexanol, 1-methoxy-2-propanol (methoxypropanol), 2-methoxy-1-propanol, 2-methoxy-1-ethanol (methoxyethanol), 2-isopropoxy-1-ethanol, acetonitrile, acetone and water. Of these solvents, primary alcohols having a SP value of not less than 9.0 (cal/cm³)^{1/2} such as methanol, ethanol, 1-propanol, 1-butanol and 1-pentanol (1-amyl alcohol), and alkoxy alcohols having a SP value of not less than 9.0 (cal/cm³)^{1/2} such as 1-methoxy-2-propanol (methoxypropanol), 2-methoxy-1-ethanol (methoxyethanol) and 2-isopropoxy-1-ethanol are more preferred.

The solubility parameter (the SP value) as used herein can be easily calculated by a simplified calculation method described below.

Equations for Calculating Solubility Parameter σ

### 1) Latent Heat of Vaporization Per 1 mol

Hb=21×(273+Tb) (unit: cal/mol), Tb: boiling point (° C.)

### 2) Latent Heat of Vaporization Per 1 mol at 25° C.

H25=Hb×{1+0.175×(Tb−25)/100} (unit: cal/mol), Tb: boiling point (° C.)

### 3) Intermolecular Binding Energy

E=H25−596 (unit: cal/mol)

4) Intermolecular Binding Energy Per 1 ml (cm³) of Solvent

E1=E×D/Mw (unit: cal/cm³), D: density (g/cm³), MW: molecular weight

### 5) Solubility Parameter (SP Value)

σ=(E1)^{1/2} (unit: cal/cm³)^{1/2}

However, in view of the fact that the dental composition of the invention is used for a dental material, the solvent is preferably a liquid having a boiling point in the range of 40 to 180° C. at normal pressure. Examples thereof include: water; alcohol solvents such as methanol, ethanol, isopropanol, n-propanol, butanol and cyclohexanol; halogen solvents such as chloroform, methylene chloride and chlorobenzene; hydrocarbon solvents such as hexane, cyclohexane, toluene and xylene; ketone solvents such as acetone, methyl ethyl ketone and cyclohexanone; ester solvents such as ethyl acetate and butyl acetate; and ether solvents; and the like, but the present invention is not limited thereto. Of these, preferred are solvents which can be evaporated after coating with relative ease, such as water, methanol, ethanol, isopropanol, n-propanol, butanol, propylene glycol monomethyl ether (PGM), 2-methoxy-1-ethanol (EGM) and acetone. These solvents may be used singly, or in combination of two or more.

The amount of the solvent contained in the composition of the invention may be selected as appropriate in view of aspects such as the physical properties of the cured product, such as a monolayer film, obtained according to the invention, and the economic efficiency.

The solvent is used in such an amount that the concentration of the solids (the total amount of the components including the compounds (I) to (III) and the “other components”, excluding the solvent) contained the composition (solids/(solids+solvent)×100) will usually be in the range of 1 wt % or more, preferably to 90 wt %, more preferably 20 to 80 wt %, and still more preferably 30 to 70 wt %.

### Fillers

The dental composition of the invention may contain a filler as required, in the case of preparing a dental composite resin, for example. The filler as used herein may be any filler generally used in the field of dentistry. In general, fillers are broadly categorized into organic fillers and inorganic fillers.

Examples of the organic fillers include fine powders of: polymethyl methacrylate, polyethyl methacrylate, methyl methacrylate-ethyl methacrylate copolymer, crosslinked polymethyl methacrylate, crosslinked polyethyl methacrylate, ethylene-vinyl acetate copolymer and styrene-butadiene copolymer; and fluorine resins such as polytetrafluoroethylene (PTFE), tetrafluoroethylene-ethylene copolymer, tetrafluoroethylene-hexafluoropropylene copolymer (FEP), polyvinylidene fluoride (PVDF), polychlorotrifluoroethylene (PCTFE) and the like.

Examples of the inorganic fillers include fine powders of: various types of glasses (comprising silicon dioxide (such as quartz, quartz glass or silica gel), alumina and silicon as major components and further containing an oxide of a heavy metal, boron, aluminum, and the like, if necessary); various types of ceramics; diatomaceous earth; kaolin; cray minerals (such as montmorillonite); activated white clay; synthetic zeolite; mica; calcium fluoride; ytterbium fluoride; calcium phosphate; barium sulfate; zirconium dioxide; titanium dioxide; hydroxyapatite; and the like. Specific examples of the inorganic fillers include: barium borosilicate glasses (such as Kimble RAY-SORB T3000, Schott 8235, Schott GM27884 and Schott GM39923), strontium boroaluminosilicate glasses (such as RAY-SORB T4000, Schott G018-093 and Schott GM32087), lanthanum glasses (such as Schott GM31684), fluoroaluminosilicate glasses (such as Schott G018-091 and Schott G018-117), and boroaluminosilicate glasses containing zirconium and/or cesium (such as Schott G018-307, G018-308 and G018-310).

It is also possible to use an organic/inorganic composite filler obtained by: adding a polymerizable monomer to an inorganic filler such as those described above in advance, and forming the resultant into a paste, followed by polymerization and curing, and then by grinding the resultant.

Further, in one of the preferred embodiments of the dental composite resin, the dental composition includes a microfiller having a particle diameter of 0.1 μm or less. Preferred materials for fillers having such a small particle diameter include silica (such as AEROSIL (trade name)), alumina, zirconia and titania. The incorporation of the inorganic filler having such a small particle diameter is advantageous in imparting a polishing smoothness to the cured product of the composite resin.

There are cases where these fillers are subjected to a surface treatment with a silane coupling agent or the like, depending on the objective. Examples of such surface treatment agent include known silane coupling agents, such as organic silicon compounds, for example, γ-methacryloxyalkyltrimethoxysilanes (the number of carbon atoms between the methacryloxy group and the silicon atom: 3 to 12), γ-methacryloxyalkyltriethoxysilanes (the number of carbon atoms between the methacryloxy group and the silicon atom: 3 to 12), vinyltrimethoxysilane, vinylethoxysilane and vinyltriacetoxysilane. The surface treatment agent is usually used at a concentration in the range of 0.1 to 20 wt %, and preferably 1 to 10 wt %, with respect to 100 wt % of the filler.

When the sustained release of fluorine ions from the surface of the cured product is desired, it is possible to add a filler capable of sustained release of fluorine ions, such as fluoroaluminosilicate glass filler, calcium fluoride, sodium fluoride, or sodium monofluorophosphate.

These fillers are used singly or in combination of two or more, as appropriate. The amount of the filler(s) added may be selected as appropriate in view of the handleability (viscosity) of the composite resin paste and/or the mechanical properties of the resulting cured product. The amount is usually 10 to 2,000 parts by weight, preferably 50 to 1,000 parts by weight, and more preferably 100 to 600 parts by weight, with respect to 100 parts by weight of the total components contained in the dental composition excluding the filler.

### Other Additives

The dental composition of the invention may include any of the following components, in addition to the components described above.

Examples of the components which can be added to impart antibacterial activity to the composition include surfactants having an antibacterial activity such as cetylpyridinium chloride and 12-(meth)acryloyloxydodecylpyridinium bromide, and photocatalytic titanium oxide.

Examples of the components which can be added to impart X-ray contrast properties to the composition include glass fillers (such as barium boroaluminosilicate glass) containing heavy metal elements such as barium, ytterbium, strontium and lanthanum; and fine powders of ytterbium fluoride and barium sulfate.

Examples of the components which can be added to adjust the viscosity or the coating properties of the composition include thickeners such as sodium polyacrylate, sodium alginate and acacia; and silica microfillers having an average particle diameter of 0.1 μm or less [such as AEROSIL (trade name), manufactured by NIPPON AEROSIL CO., LTD.].

### <Preparation Method>

The dental composition of the invention can be obtained by mixing the compound (I), the compound (II) and the surfactant (III); as well as the “other components” as required.

The dental composition of the invention can be obtained by mixing all these components at the same time; or alternatively, it can be obtained by preparing a polymerizable composition which contains the compound (I) and the compound (II) but not the surfactant (III) nor a polymerization initiator in advance, and then adding the surfactant (III), and other components such as the polymerization initiator as required, to the polymerizable composition.

The dental composition containing little or no solvent may be obtained by mixing the compound (I), the compound (II), the surfactant (III) and the like without using a solvent at all; or alternatively, by preparing a diluted dental composition containing a solvent, and then removing the solvent from the diluted dental composition under appropriate conditions where the reaction of the compound (I) and the compound (II) are not elicited.

### [Dental Cured Product]

The dental cured product of the invention can be obtained by curing the above descried dental composition of the invention. Since the dental cured product of the present invention has a certain level of hydrophilicity, it may be referred to as a “hydrophilic dental cured product” or “hydrophilic cured product” in the present specification. Further, when the context obviously indicates that the dental cured product of the invention is being referred to, it may be simply referred to as a “cured product” for convenience.

The shape of the resulting dental cured product (hydrophilic dental cured product) of the invention is not particularly limited. However, in a preferred and exemplary embodiment of the present invention, the dental cured product has the form of a monolayer film. Such a monolayer film may be referred to as a “dental monolayer film” in the present invention.

### <Dental Monolayer Film>

The dental monolayer film of the invention is composed of a crosslinked resin obtained by curing the above described dental composition, namely, the hydrophilic dental cured product. In other words, the dental monolayer film of the invention is a monolayer film composed of the hydrophilic dental cured product.

In the present specification, such a dental monolayer film may be simply referred to as a “monolayer film” for convenience.

In the present invention, the monolayer film has a concentration gradient (the ratio of the concentrations of anions) (Sa/Da) of hydrophilic group(s) of 1.1 or more, preferably, 1.2 or more, more preferably 1.3 or more, and still more preferably 1.5 or more, wherein the concentration gradient (Sa/Da) is obtained from the concentration of at least one type of hydrophilic groups selected from anionic hydrophilic groups, cationic hydrophilic groups and hydroxyl group, at the surface (Sa) of the monolayer film, and at the ½ point (deep concentration) (Da) in the thickness of the monolayer film.

The monolayer film of the invention, which contains the hydrophilic groups as described above, is usually formed as a coating on at least one surface of an object such as a tooth surface or a dental prosthesis. In the monolayer film, the hydrophilic groups are distributed from the deep portion to the surface of the film on the side of the tooth surface, the dental prosthesis or the like. In particular, the concentration distribution (the gradient (the ratio of the concentrations of hydrophilic groups) (Sa/Da)) is such that the concentration is higher on the top surface of the monolayer film in contact with the air.

The reason for the occurrence of such a distribution is thought to be as follows: when the dental composition is coated on an object such as a tooth surface or a dental prosthesis and cured by applying heat, radiation or the like as will be described in the following “Formation method”, the concentration of at least one type of hydrophilic groups selected from anionic hydrophilic groups, cationic hydrophilic groups and hydroxyl group is enriched (concentrated) at the surface in contact with the air, and after the occurrence of such enrichment, a monolayer film composed of the cured product of the dental composition is formed.

As described above, since the hydrophilic groups are present at a high concentration at the surface of the monolayer film constituting the dental material of the invention, the monolayer film exhibits excellent antifouling properties, self-cleaning properties or the like.

The gradient (the ratio of the concentrations of hydrophilic groups) is determined by a process in which: a portion of a predetermined sample of the monolayer film is cut out obliquely; the concentrations of groups including anionic hydrophilic groups (such as sulfo group, carboxyl group and phosphate group), cationic hydrophilic groups (such as quaternary ammonium groups) and hydroxyl group are measured at the surface of the monolayer film in contact with the air and at the ½ point in the thickness of the monolayer film, as the intensities of the fragment ions, using a time-of-flight secondary ion mass spectrometer (TOF-SIMS); and the gradient is obtained from the ratio of the (relative) intensities of the fragment ions.

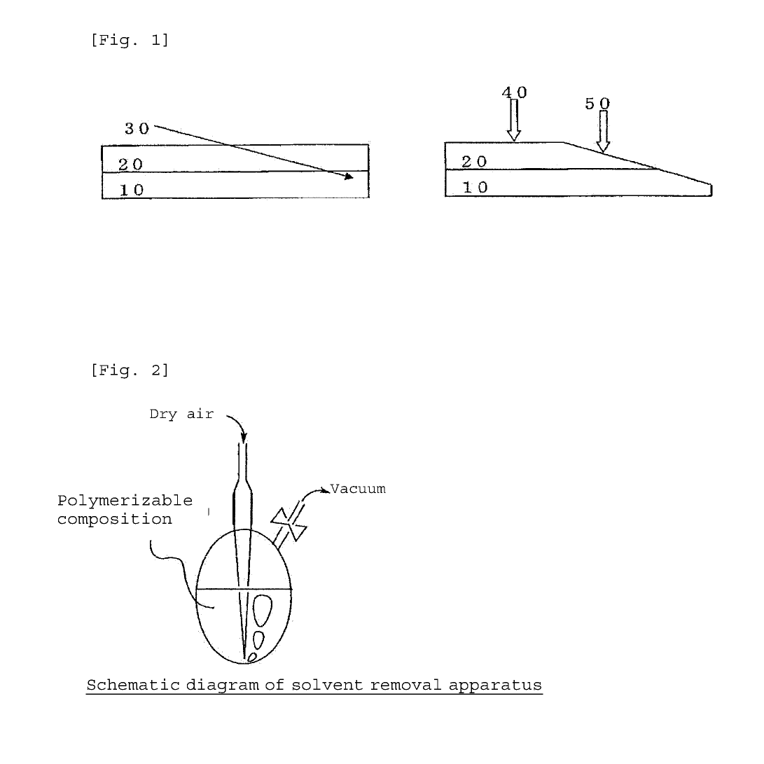

For example, a portion of the sample is cut off obliquely as shown in FIG. 1. Then, using a time-of-flight secondary ion mass spectrometer (TOF-SIMS), the concentrations of the fragment ions derived from a hydrophilic compound having hydrophilic groups, such as sulfo group, carboxyl group, phosphate group, quaternary ammonium groups and hydroxyl group, are measured, at the top surface (Sa) and at the above described ½ point (Da) of the film. Based on the thus obtained values, the ratio of the concentrations of the hydrophilic groups derived from the hydrophilic compound that are present at the top surface of the film in contact with the air and at the midpoint between the top surface and the bottom surface of the film, namely the gradient of the hydrophilic group concentration (Sa/Da), can be obtained.

The monolayer film constituting the dental material of the invention usually has a water contact angle of 50° or less, and preferably 30° or less.

The monolayer film having a water contact angle of not more than the above described values has a high hydrophilicity, and serves as an excellent hydrophilic material which is highly compatible (wettable) with water. Thus, the monolayer film is useful as, for example, an antifogging material, an antifogging film, a self-cleaning coat or the like. When used as a self-cleaning coat, for example, water penetrates in between the dirt and the coating surface to lift and remove the dirt, thereby exhibiting an excellent antifouling effect. Further, the capability of the hydrophilic monolayer film to allow water to spread leads to an increased evaporation area and thus provides an enhanced evaporation speed, namely, quick drying.

When the monolayer film of the invention has a water contact angle of not more than the above described upper limit, it is particularly preferably used as an antifouling material. The monolayer film usually has a water contact angle of 0° or more.

The curing of the composition containing the surfactant (III) allows the hydrophilic groups derived from the compound (I) to be enriched (concentrated) at the surface of the resulting monolayer film, in the absence of a solvent. This allows for producing a highly hydrophilic cured product under a wider range of conditions. At the same time, the transparency of the cured products is relatively increased, probably due to the compatibilizing effect that prevents the separation of the compound (I) and the compound (II). A conventional method (such as one described in WO 2007/064003) allows the enrichment (concentration) of hydrophilic groups at the surface in synchronism with the evaporation of a polar solvent. Such a method has been incapable of producing a cured product with an enriched (concentrated) hydrophilic group concentration at the surface, from a composition including a general hydrophilic polymerizable compound (for example, hydrophilic polymerizable compounds except for those described in the claims of WO 2007/064003). However, the incorporation of the surfactant (III) makes it possible to produce a hydrophilic cured product with an increased hydrophilic group concentration at the surface, even from the composition including such a general hydrophilic polymerizable compound. Further, a cured product, such as a monolayer film, obtained from the composition including the hydrophilic compound described in the above publication, tends to have a higher concentration (a higher enrichment) of the hydrophilic groups at the surface, and thus tends to exhibit higher hydrophilicity. Still further, although it has been difficult to obtain the enrichment with the use of a low-polarity solvent having a solubility parameter (SP value) of less than 9.3, the inventive technique makes it relatively easy to obtain a hydrophilic cured product in which hydrophilic groups are enriched (concentrated) at the surface, even with the use of such a solvent. Accordingly, the invention allows for the production of a monolayer film which is composed of the cured product as described above and which has a high hydrophilicity and transparency, with more ease than ever and with the use of a wide variety of materials, as well as the application of the monolayer film as a dental material.

The monolayer film of the invention usually has a film thickness in the range of 0.0001 to 500 μm, preferably 0.05 to 500 μm, more preferably 0.1 to 300 μm, more preferably, 0.1 to 100 μm, still more preferably 0.5 to 100 μm, still more preferably 1 to 50 μm, and particularly preferably 2 to 30 μm.

### <Formation Method>

The method of forming the dental cured product of the invention, such as the monolayer film, is not particularly limited. For example, the dental cured product may be formed by a method in which the dental composition, which is a polymerizable composition, is coated on the surface of a substrate, and the solvent contained in the polymerizable composition is removed, as required, followed by curing the polymerizable composition. The monolayer film can be suitably formed, according to the above described method.

The coating of the dental composition can be carried out by a conventional method, such as coating using a brush, dip coating, spray coating, spin coating or bar coating. When the dental prosthesis according to the present invention to be described later is produced, the coating can be suitably carried out, for example, by dip coating, as will be shown in the Examples to be described later.

Further, it is also possible to obtain the dental prosthesis of the invention by forming a monolayer film on a polymer film using any of the conventionally known coating methods described above, and then by laminating the resulting films.

### Substrate

In the present invention, the substrate onto which the dental composition of the invention will be coated is a tooth or a dental prosthesis. Examples of the dental prosthesis to be used as a substrate include inlays, crowns, bridges, partial dentures, complete dentures, implants and the like. In the present invention, the prosthesis may be a dental restorative material, a mouthpiece, an orthodontic device or an intraoral device. Further, the substrate may be an artificial tooth or a natural tooth. Specific examples of the material for these include a dentin, and various types of metals, ceramics, resins, composite resins and the like which can be generally used for a dental prosthesis. Examples of the ceramics which can be used as the substrate in the present invention include glasses, silica, metal oxides and the like. The ceramics may also be the same as those exemplified above as the inorganic fillers. Examples of the resins which can be used as the substrate in the present invention include: various types of acrylic resins such as polyacrylates, and polymethacrylates, for example, polymethyl methacrylate (PMMA); materials obtained by copolymerizing acrylates with various types of monomers; materials obtained by copolymerizing methacrylates such as methyl methacrylate (MMA) with various types of monomers; polycarbonates; polyethylene terephthalates; polyethylenes; polypropylenes; polystyrenes; polyurethane resins; epoxy resins; vinyl chloride resins; silicone resins; polyether ether ketone (PEEK) resins, polyether ketone (PEK) resins, polyether ketone ketone (PEKK) resins, polyether ether ketone ketone (PEEKK) resins and polyether ketone ether ketone ketone (PEKEKK) resins; polysulfones (PSU), polyethersulfones (PES) and polyphenylsulfones (PPSU); and various types of polymer alloy materials containing the above mentioned resins; and the like. The resins may also be the same as those exemplified above as the organic fillers. Further, the composite resins may be made of the same materials as those described above for the organic/inorganic composite filler.

When the dental composition of the invention is used as a coating agent for a tooth surface or a dental prosthesis, the tooth surface or the dental prosthesis may be subjected to various types of pretreatments in order to increase the adhesion between the surfaces thereof with the composition. For example, in the case of coating the dental composition on a natural tooth intraorally, an etching treatment may be carried out with an aqueous phosphoric acid solution, an aqueous oxalic acid solution, an aqueous citric acid solution, an aqueous tartaric acid solution or an aqueous ferric chloride solution; or an adhesive primer or a bonding agent containing a functional monomer having adhesive properties may be coated on the dentin in advance.

In cases where the substrate used as a dental prosthesis is made of a material such as a ceramic, a composite resin or a metal, the substrate may be subjected to a sandblasting treatment or a treatment with a primer containing a silane coupling agent or phosphoric acid monomer. Further, when the substrate is made of a resin such as polymethyl methacrylate (PMMA) or polycarbonate, for example, as in the case of a denture base resin, a solvent such as methylene chloride, acetone or methyl isobutyl ketone may be coated to treat the substrate.

In order to activate the substrate surface, the surface of the substrate used in the present invention may be subjected to physical or chemical treatments, as required, such as corona treatment, ozone treatment, low-temperature plasma treatment using a gas such as oxygen or nitrogen, glow discharge treatment, oxidation treatment with chemicals and flame treatment. Instead of or in addition to such treatments, the substrate surface may be subjected to a primer treatment, undercoating treatment or anchor coating treatment.

Examples of the coating agents used in the primer treatment, the undercoating treatment and the anchor coating treatment include coating agents containing, as the main components of vehicles, resins such as polyester resins, polyamide resins, polyurethane resins, epoxy resins, phenolic resins, (meth)acrylic resins, polyvinyl acetate resins, polyolefin resins including polyethylenes and polypropylenes as well as copolymers thereof and modified resins thereof, and cellulose resins. The coating agents may be any of solvent-based coating agents and aqueous coating agents.

Of the coating agents, preferred are: modified polyolefin coating agents, ethyl vinyl alcohol coating agents, polyethyleneimine coating agents, polybutadiene coating agents, polyurethane coating agents; polyester polyurethane emulsion coating agents, polyvinyl chloride emulsion coating agents, urethane acrylic emulsion coating agents, silicon acrylic emulsion coating agents, vinyl acetate acrylic emulsion coating agents, acrylic emulsion coating agents; styrene-butadiene copolymer latex coating agents, acrylonitrile-butadiene copolymer latex coating agents, methyl methacrylate-butadiene copolymer latex coating agents, chloroprene latex coating agents, rubber latex coating agents containing polybutadiene latex, polyacrylate latex coating agents, polyvinylidene chloride latex coating agents, polybutadiene latex coating agents, and coating agents which include latexes or dispersions resulting from the carboxylic acid modification of the resins contained in the above latex coating agents.

For example, these coating agents can be coated by methods such as a gravure coating method, a reverse roll coating method, a knife coating method and a kiss-roll coating method, and the amount to be coated on the substrate is usually 0.05 g/m² to 10 g/m², as measured in the dry state.

Of the coating agents, polyurethane coating agents are more preferable. The polyurethane coating agents have urethane bonds in the main chains or side chains of the resins present in the coating agents. For example, the polyurethane coating agents contain a polyurethane obtained by reacting a polyol such as a polyester polyol, a polyether polyol or an acrylic polyol, with an isocyanate compound.

Of the polyurethane coating agents, those polyurethane coating agents which are obtained by mixing a polyester polyol such as a condensed polyester polyol or a lactone-based polyester polyol with an isocyanate compound such as tolylene diisocyanate, hexamethylene diisocyanate or xylene diisocyanate are preferable because of their excellent adhesion.

The polyol compounds and the isocyanate compounds may be mixed with each other by any methods without limitation. The mixing ratio is not particularly limited. When, however, the amount of the isocyanate compound is excessively small, curing failures may be caused. Thus, the equivalent ratio of the OH groups of the polyol compound to the NCO groups of the isocyanate compound is preferably in the range of 2/1 to 1/40.

The substrates in the invention may have a surface that has been treated by the aforementioned surface activation.

The substrate whose surface is coated with a monolayer film composed of the hydrophilic cured product of the invention, as described above, may be used as a laminate including the substrate and the monolayer film.

In the present specification, the dental prosthesis used as a substrate may be referred to as a “substrate prosthesis”, in order to distinguish it from the dental prosthesis according to the invention to be described later. As will be described later, the “substrate prosthesis” on the surface of which a monolayer film composed of the hydrophilic cured product of the invention is formed, can be used as the dental prosthesis according to the invention to be described later.

### Solvent Removal

When the dental composition of the invention contains the solvent, it is preferred that the solvent in the composition coated on an object such as a tooth surface or a dental restorative material be sufficiently removed by heating or the like, before carrying out the curing to be described later. Insufficient removal of the solvent from the composition results in less movement of the hydrophilic groups derived from the compound (I) (at least one type of hydrophilic groups selected from anionic hydrophilic groups, cationic hydrophilic groups and hydroxyl group) toward the surface of the coating in contact with the air. As a result, the properties such as the hydrophilicity of the resulting monolayer film tend to decrease. Even if the hydrophilic groups have successfully moved to the surface of the coating in contact with the air, the residual solvent in the composition causes a repelling interaction with the air (which is hydrophobic) present at the surface in contact with the air, thereby tends to facilitate the movement of the hydrophilic groups toward the interior of the coating. Consequently, there are cases where the enrichment of the hydrophilic groups at the surface in contact with the air of the resulting monolayer film may be insufficient, and where the hydrophilicity of the film is decreased. In addition, the adhesion between the monolayer film and the tooth surface, the dental restorative material or the like also tends to decrease. Thus, a lesser amount of the residual solvent in the composition immediately before the curing tends to provide more favorable results. The amount of the residual solvent is usually 10 wt % or less, preferably 5 wt % or less, more preferably 3 wt % or less, and still more preferably 1 wt % or less.

The temperature during the removal of the solvent may be selected as appropriate, but it is usually in the range of room temperature to 200° C., preferably 30 to 150° C., and more preferably to 120° C.

The removal of the solvent from the composition may be performed for an appropriately selected period of time. However, a shorter time tends to be preferred in terms of productivity. For example, the solvent removal is usually carried out by drying for 30 minutes or less, preferably 10 minutes or less, and more preferably 5 minutes or less. The solvent removal may take place in an atmosphere of air or an inert gas such as nitrogen. An atmosphere with a lower humidity tends to provide preferred results such as less deterioration in the appearance (for example, occurrence of orange peel surface, or a decrease in transparency) of the resulting monolayer film. Specifically, the humidity in the atmosphere is preferably 80% or less, more preferably 65% or less, and still more preferably 55% or less.

When the solvent is removed by the application of wind, the wind speed is preferably not more than 30 m/sec, more preferably in the range of 0.1 to 30 m/sec, still more preferably in the range of 0.2 to 20 m/sec, and particularly preferably in the range of 0.3 to 10 m/sec.

The pressure during the solvent removal is not particularly limited. Normal pressure or a reduced pressure is relatively preferable. A slightly increased pressure may also be used.

### Curing

The hydrophilic dental cured product according to the present invention is obtained by coating the above described dental composition on the substrate or the like, and then by curing the resultant. When the dental composition contains the solvent, the curing can be carried out after coating the composition on the substrate or the like and further carrying out the above described solvent removal, as required.

The curing of the dental composition is carried out by allowing the compound (I) to be copolymerized with the compound (II) in the presence of the surfactant (III).

The curing method is not particularly limited, and the curing may be carried out using heat or radiation, or alternatively, using both heat and radiation.

The dental composition of the invention can be cured under appropriate conditions depending on the polymerization method suited to the above described polymerization initiators.

The curing may be carried out in an air atmosphere. However, it is preferably carried out in an atmosphere of an inert gas such as nitrogen, because it allows for reducing the curing time.

When the curing is carried out using heat, a thermal radical generator such as an organic peroxide is usually added to the dental composition, and the mixture is heated at a temperature in the range of room temperature to 300° C. or less.

When the curing is carried out using radiation, an energy ray with a wavelength in the range of 0.0001 to 800 nm can be used as the radiation. Such radiations are categorized into α-rays, β-rays, γ-rays, X-rays, electron beams, UV light and visible light. An appropriate radiation may be selected depending on the chemical composition of the mixture. Of the radiations, UV light is preferred. The output peak of the UV light is preferably in the range of 200 to 450 nm, more preferably in the range of 230 to 445 nm, still more preferably in the range of 240 to 430 nm, and particularly preferably in the range of 250 to 400 nm. The use of UV light having an output peak in the above range is advantageous in that defects such as yellowing and thermal deformation during the curing are reduced, and the curing can be completed in a relatively short period of time even when the composition contains a UV absorber.

When the composition contains the UV absorber and/or the hindered amine stabilizer, it is preferable to use UV lights having an output peak in the range of 250 to 280 nm or 370 to 430 nm.

On the other hand, when the composition contains a photopolymerization initiator which absorbs visible light, such as camphorquinone or DAROCUR TPO, it is also possible to use visible light as the radiation for the curing. In this case, visible light having an output peak in the range of 400 to 500 nm is preferably used.

When the polymerization of the composition is carried out using radiation, the polymerization may be carried out by a process in which the composition is coated on a substrate or the like, the resultant is dried as necessary, and then the coating layer is covered with a covering material (such as a film), followed by irradiation, in order to prevent the inhibition of polymerization by oxygen. The coating layer is preferably covered with the covering material in close contact, so that air (oxygen) will not be entrapped between the coating layer and the covering material.

By blocking oxygen, for example, there are cases where the amount of the (photo)polymerization initiator and the radiation dose can be reduced.

The covering materials are not particularly limited, and various materials can be used in various forms as long as oxygen can be blocked. Films are preferable in terms of handling. Of the films, transparent films allowing for easy radiation polymerization are preferable. The thickness of the films is usually in the range of 3 to 200 μm, preferably 5 to 100 μm, and more preferably 10 to 50 μm.

Examples of the film materials suitably used as the covering materials include vinyl alcohol polymers such as polyvinyl alcohols (PVAs) and ethylene.vinyl alcohol copolymers, polyacrylamides, polyisopropylacrylamides, polyacrylonitriles, polycarbonates (PCs), polymethyl methacrylates (PMMAs), polyethylene terephthalates (PETs), polystyrenes (PSs) and biaxially oriented polypropylenes (OPPs).

Electron beams in the range of 0.01 to 0.002 nm are preferable as the radiations because the polymerization can be completed in a short time, although such apparatuses are expensive.

For example, in the case of the dental composition of the invention containing a photopolymerization initiator which initiates polymerization when visible light is irradiated, a desired cured product can be obtained by forming the dental composition into a predetermined shape, and then subjecting the resultant to visible light irradiation using a known light irradiation apparatus for a predetermined period of time. Conditions such as irradiation time and irradiation strength may be changed as appropriate depending on the curing performance of the dental composition. Further, the heat treatment of the cured product cured by irradiation of light such as visible light, under appropriate conditions, allows for improving the mechanical properties of the cured product.

It is preferred that the dental composition be cured by irradiation of light in terms of ease of operation.

### [Application of Dental Composition and Dental Cured Product]

The above described dental composition of the invention can be suitably used as a dental material in the form of the dental cured product. In the present invention, preferred examples of the dental material include the dental prosthesis including the dental monolayer film.

The dental prosthesis according to the present invention includes a monolayer film obtained by curing the above described dental composition of the invention. Specifically, the dental prosthesis according to the present invention includes the dental prosthesis (namely, the “substrate prosthesis”) described in the section of “Substrate” above, and the monolayer film obtained by curing the dental composition of the invention. In an exemplary embodiment of the present invention, the monolayer film is disposed in such a manner that the film covers a part or the entirety of the surface of the substrate prosthesis. The dental prosthesis according to the present invention as described above is obtained by using the dental prosthesis (namely, the “substrate prosthesis”) described in the section of “Substrate” above as a substrate, and applying the method described in the section of “Formation Method” above to the substrate prosthesis.

Specific examples of the application of dental composition of the invention include dental composite resins such as dental composite filler materials, tooth crown materials and cementing materials; dental adhesive agents such as orthodontic adhesive agents, cavity-coating adhesive agents and tooth fissure sealants; denture base materials; mucosa conditioning agents for denture base; fissure sealants; coating agents for tooth surface or dental prosthesis; surface glazes, and the like. Since it is possible to form a hard and thin coating after the curing when the dental composition contains a solvent as described above, the dental composition can be suitably used in various types of coating applications such as fissure sealants; dental coating agents, surface stains and surface glazes for tooth surface or dental prosthesis; hypersensitivity inhibitors; dental manicures; and the like.

The method of using the dental cured product of the invention is not particularly limited, and any method generally known as a method for using a dental material can be used. For example, when the dental composition of the invention is used as a composite resin for filling tooth decay cavities, the objective can be achieved by filling the dental composition in an intraoral cavity and then photocuring the composition using a known light irradiation apparatus. Further, when the dental composition is used as a composite resin for tooth crown, a desired crown material can be obtained by forming the dental composition into an appropriate shape and then photocuring the resultant using a known light irradiation apparatus, followed by heat treatment under predetermined conditions.

### EXAMPLES

The present invention will now be described in further detail with reference to Examples. However, the present invention is not limited thereto.

In the present specification, the “solid concentration” as used in the description hereinbelow refers to the ratio of the components other than solvent with respect to the total amount of the composition.

Properties of films in the invention were evaluated as described below.

### <Measurement of Ratio of Concentrations of Anions>

A portion of a sample was cut out obliquely as illustrated in FIG. 1. The sample was then analyzed using a time-of-flight secondary ion mass spectrometer (TOF-SIMS), to measure the concentration of anions (Sa) at the above described top surface, and the concentration of anions (Da) at the above described mid-point. Based on the thus obtained values, the ratio of the concentration of anions at the top surface of the film in contact with the air and the concentration of anions at the mid-point between the bottom surface and the top surface of the film, namely, the gradient of the concentration of anions (Sa/Da) was obtained.

### (Analyzer and Measurement Conditions)

TOF-SIMS: TOF-SIMS 5 manufactured by ION.TOF

Primary ions: Bi₃²⁺ (accelerating voltage 25 kV)

Measurement area: 350 to 500 μm²

In the measurement, a neutralizing gun for correcting electric charges was used.

### (Preparation and Analysis of Sample)

As shown in FIG. 1, a portion of a sample consisting of a substrate 10 and a coating layer 20 disposed on the surface of the substrate 10 was cut out precisely obliquely in a cutting direction 30. Thereafter, the sample was cut into a piece of an approximately 10×10 mm², and a mesh was placed on the measurement surface. The resulting sample was then fixed to a sample holder, and the concentration of anions was measured at a surface 40 of the coating layer in contact with air and at an inner portion 50 of the coating layer located inside the film (at the ½ point in the thickness of the film, a surface of the inside of the coating layer in contact with the substrate 10), using the time-of-flight secondary ion mass spectrometer (TOF-SIMS).

### (Evaluation)

The evaluation was carried out according to the equation below. The ion concentrations at the respective measurement points were relative intensities (relative to the total amount of the detected ions).

Sa/Da (ratio of concentrations of anions, gradient)=concentration of anions at surface 40 of coating layer/concentration of anions at ½ point in the film thickness of coating layer 20

### <Measurement of Water Contact Angle>

The water contact angle was measured at 3 points in each of the samples using a water contact angle meter, model CA-V, manufactured by Kyowa Interface Science Co., Ltd. The average of the thus obtained values was defined as the contact angle.

### <Measurement of Color Difference>

A coating test specimen (size: 20 mm×70 mm×2 mm thickness) was immersed in a lipophilic colorant (Bon Curry Gold, medium hot (solid ingredients were removed) manufactured by Otsuka Foods Co., Ltd.) and maintained in that state at 40° C. for 6 hours. After washing the test specimen with running water, the test specimen was immersed in distilled water, and maintained at that state at room temperature for 12 to 18 hours. The above described operation was repeated 6 times, and after the 7th washing with running water, the colorimetric values of the test specimen were measured using a spectrocolorimeter (CM-2500d, manufactured by Konica Minolta, Inc.; light source: c, colorimetric visual field: 2°). The colorimetric values of the test specimen before being immersed in the colorant were used as standards, and the color difference ΔE*ab after the immersion was obtained. A larger numerical value of the ΔE*ab indicates that the test specimen has a poorer contamination resistance.

The color difference ΔE*ab is calculated according to the following equation, using the colorimetric values (L*0, a*0, b*0) before the immersion in the colorant, and colorimetric values (L*1, a*1, b*1) after the immersion in the colorant, as indicated in the L*a*b* color system.

ΔE*ab=[(L*1−L*0)²+(a*1−a*0)²+(b*1−b*0)²]^{1/2}

### Preparation Example 1

### (Preparation of Polymerizable Composition 1)

A uniform polymerizable composition 1 having a solid concentration of 80 wt % was prepared in accordance with the formulation shown in Table 1 below. The symbols in Table 1 indicate compounds represented by the formulas shown below.

TABLE 1
Composition of polymerizable composition 1

| Materials | Amount (g) | Concentration (wt %) | Note |
|---|---|---|---|
| SPA-K | 1.50 | 1.18 | Compound (I) |
| A-GLY-9E | 20.00 | 15.75 | Compound (II) |
| A-9530 | 80.00 | 62.99 | Compound (II) |
| S-EED | 0.10 | 0.08 | Stabilizer |
| Methanol | 25.40 | 20.00 | Solvent |
| Total | 127.00 | 100.00 | |
| Solid | | 80 | |
| concentration | | | |
| (wt %) | | | |

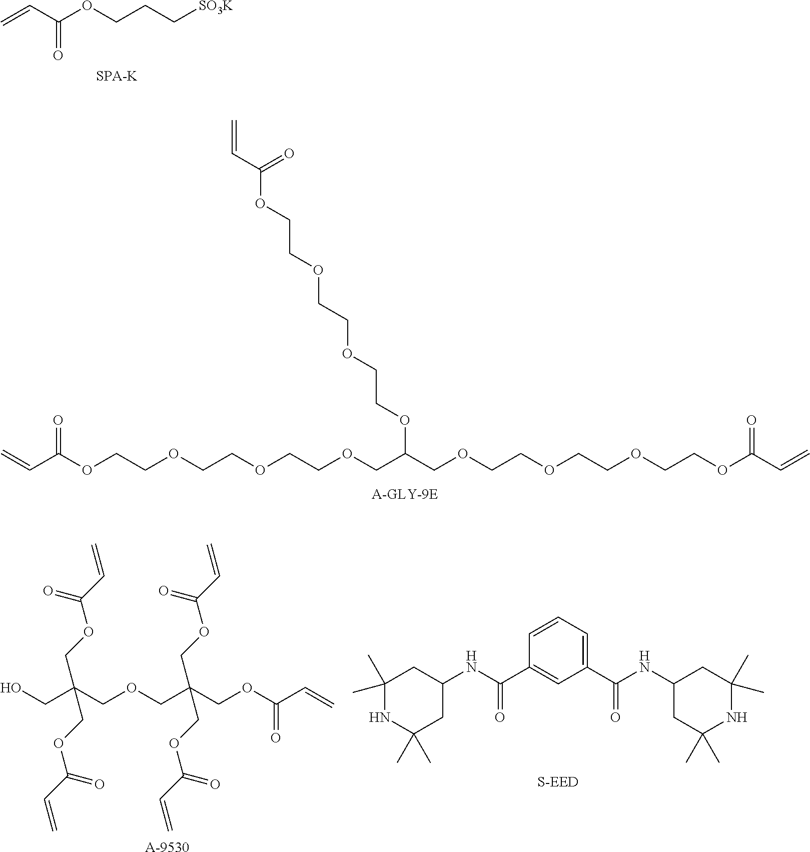

### Preparation Example 2

### (Preparation of Polymerizable Composition 2)

A uniform polymerizable composition 2 having a solid concentration of 80 wt % was prepared in accordance with the formulation shown in Table 2 below. The symbols in Table 2 indicate compounds represented by the formulas shown below.

TABLE 2
Composition of polymerizable composition 2

| Materials | Amount (g) | Concentration (wt %) | Note |
|---|---|---|---|
| SPA-K | 1.70 | 1.34 | Compound (I) |
| EA5721 | 20.00 | 15.72 | Compound (II) |
| A-BPE-10 | 10.00 | 7.86 | Compound (II) |
| U-15HA | 70.00 | 55.03 | Compound (II) |
| S-EED | 0.10 | 0.08 | Stabilizer |
| Methanol | 25.40 | 19.97 | Solvent |
| Total | 127.20 | 100.00 | |
| Solid | | 80 | |
| concentration | | | |
| (wt %) | | | |

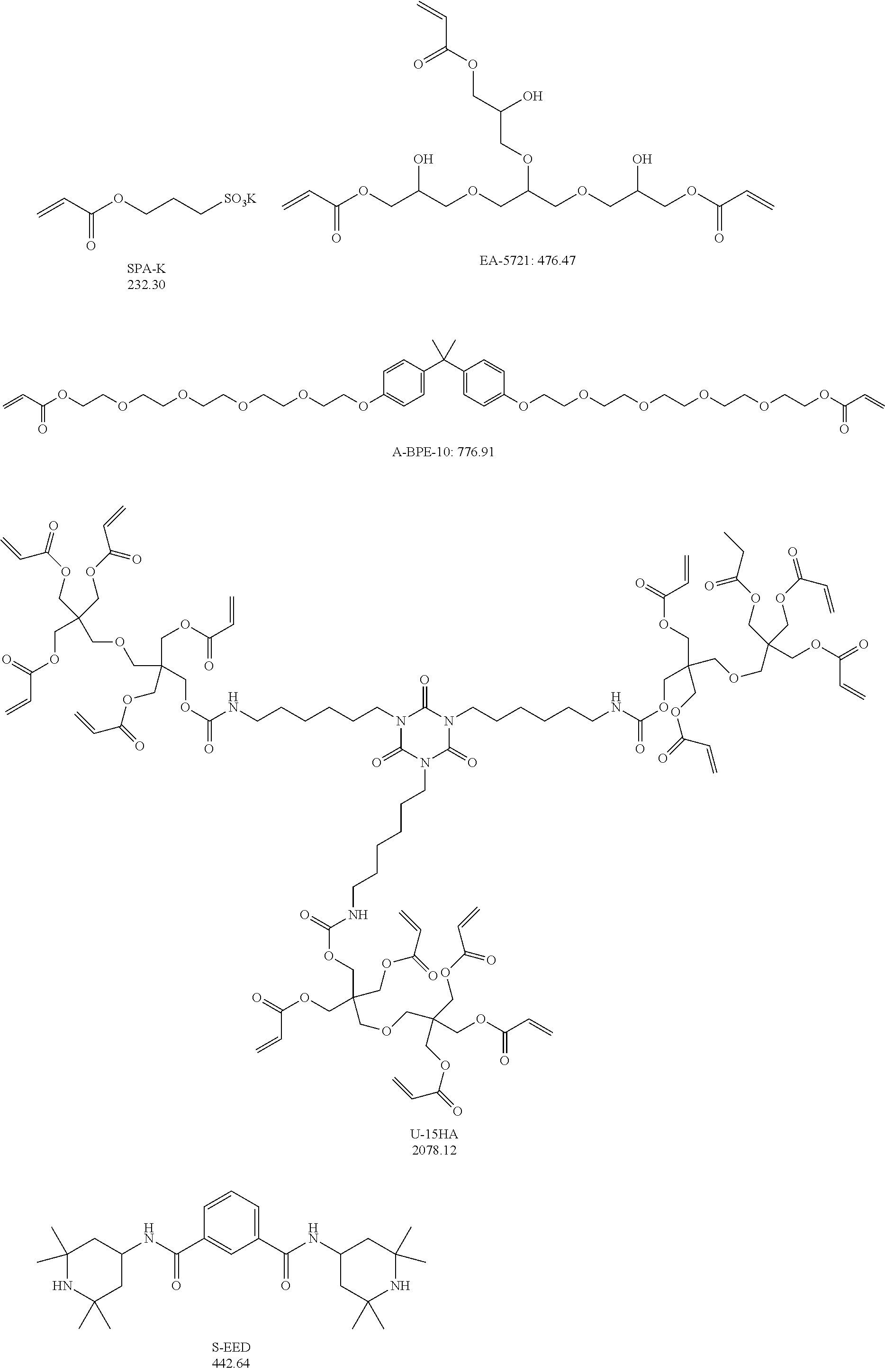

### Preparation Example 3

### (Preparation of Polymerizable Composition 3)

A uniform polymerizable composition 3 having a solid concentration of 80 wt % was prepared in accordance with the formulation shown in Table 3 below. The symbols in Table 3 indicate compounds represented by the formulas shown below.

TABLE 3
Composition of polymerizable composition 3

| Materials | Amount (g) | Concentration (wt %) | Note |
|---|---|---|---|
| SPA-K | 1.70 | 1.33 | Compound (I) |
| EA5721 | 20.00 | 15.70 | Compound (II) |
| A-BPE-10 | 10.00 | 7.85 | Compound (II) |
| U-15HA | 70.00 | 54.97 | Compound (II) |
| S-EED | 0.10 | 0.08 | Stabilizer |
| Water | 2.55 | 2.00 | Solvent |
| 99.8% Ethanol | 23.00 | 18.06 | Solvent |
| Total | 127.35 | 100.00 | |
| Solid | | 80 | |
| concentration | | | |
| (wt %) | | | |

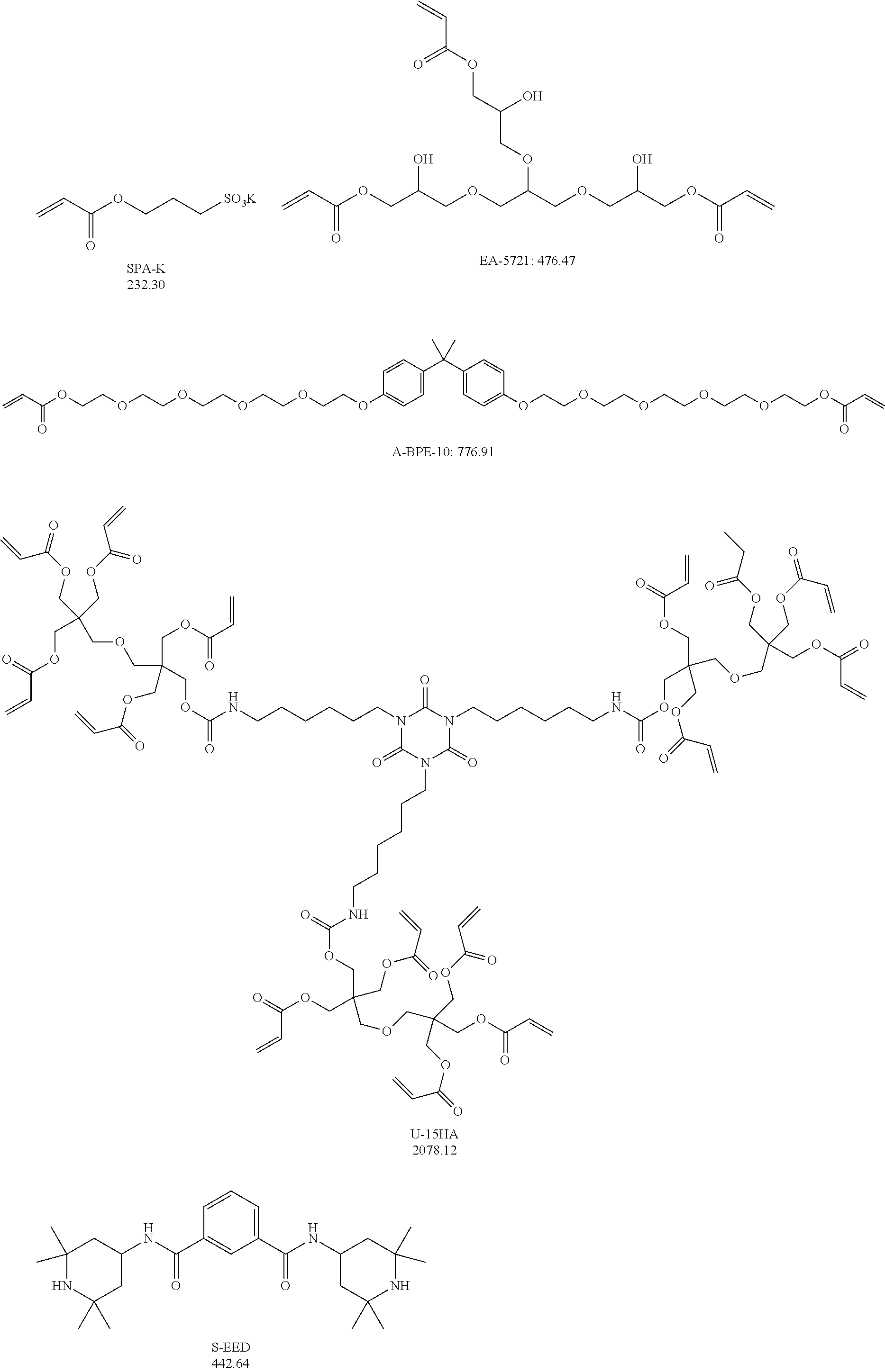

### Preparation Example 4-1

### (Preparation of Solution of Surfactant (III): DS-Na-1)

Using a homomixer (ROBOMIX (registered trademark), S-model; manufactured by PRIMIX Corporation), 10 g of sodium distearylsulfosuccinate (hereinafter, abbreviated as DS-Na) represented by the following formula, 30 g of water and 60 g of 1-methoxy-2-propanol (hereinafter, abbreviated as PGM) were mixed and stirred at 15,000 rpm for 3 minutes, to give a mixed solution of DS-Na having a solid concentration of 10 wt %.

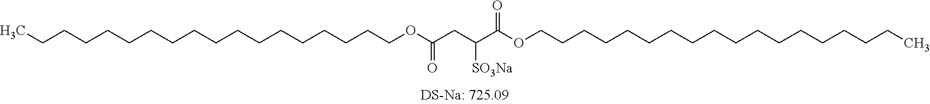

### Preparation Example 4-2

### (Preparation of Solution of Surfactant (III): DS-Na-2)

Using a homomixer (ROBOMIX (registered trademark), S-model), 10 g of sodium distearylsulfosuccinate (hereinafter, abbreviated as DS-Na), 64 g of ethanol and 26 g of water were mixed and stirred at 15,000 rpm for 3 minutes, to give a mixed solution of DS-Na having a solid concentration of 10 wt %.

### Preparation Example 4-3

### (Preparation of Solution of Surfactant (III): DT-Na)

The same operation as in Preparation Example 4-2 was carried out except that DS-Na was replaced by sodium ditridecanylsulfosuccinate (hereinafter, abbreviated as DT-Na) represented by the following formula, to give a mixed solution of DT-Na having a solid concentration of 10 wt %.

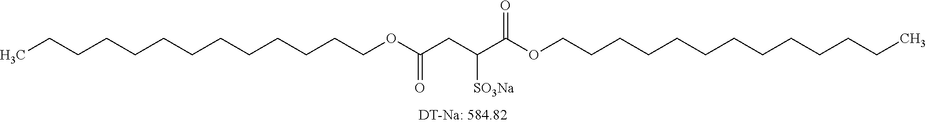

### Preparation Example 4-4

### (Preparation of Solution of Surfactant (III): DH-NH4)

Using a homomixer (ROBOMIX (registered trademark), S-model), 10 g of ammonium dihexylsulfosuccinate (hereinafter, abbreviated as DH-NH4) represented by the following formula, 70 g of ethanol and 20 g of water were mixed and stirred at 15,000 rpm for 3 minutes, to give a mixed solution of DH-NH4 having a solid concentration of 10 wt %.

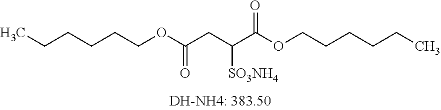

### Preparation Example 4-5

### (Preparation of Solution of Surfactant (III): LS-Na)

The same operation as in Preparation Example 4-2 was carried out except that DS-Na was replaced by sodium dodecyl sulfate (also referred to as sodium lauryl sulfate, hereinafter abbreviated as LS-Na) represented by the following formula, to give a mixed solution of LS-Na having a solid concentration of 10 wt %.

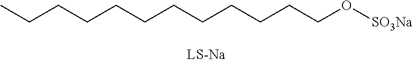

### <Pretreatment of Substrate>

A transparent acrylic plate (CLAREX-001; manufactured by Nitto Jushi Kogyo Co., Ltd.), used as a substrate to be coated, was pretreated as follows before use.

The substrate was immersed in a mixed solution of acetone and IPA (isopropyl alcohol) (at a weight ratio of 1:1) for 5 minutes, and then retrieved from the solution and subjected to air blowing. Then the substrate was dried in a fan dryer controlled at 40° C. for 5 minutes, to be used for the coating.

### Coating on Substrate and Evaluation

### Example 1

A quantity of 100 g of the polymerizable composition 1 obtained in Preparation Example 1 and having a solid concentration of 80 wt %, 0.8 g (0.1 wt % with respect to the total weight of the compound (I) and the compound (II)) of the solution of DS-Na-1 (the solution of the surfactant (III)) obtained in Preparation Example 4-1 and having a solid concentration of 10 wt %, 62 g of methanol as a diluting solvent, and 2.4 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 50 wt %. A transparent acrylic plate (CLAREX-001; manufactured by Nitto Jushi Kogyo Co., Ltd.) pretreated in advance according to the method described in the above mentioned section of the “Pretreatment of substrate” was immersed in the thus obtained solution, and pulled up at 1 mm/sec, to coat the solution on the substrate surface. The resultant was placed in a hot air dryer controlled at 50 to 60° C. for 5 minutes, to remove the solvent contained in the coating.

After sufficiently removing the solvent, the sample was subjected to UV irradiation by being passed through a UV conveyor (high pressure mercury lamp, 160 W/cm, height: 19 cm, conveyor speed: 5 m/min, intensity of illumination: 200 mW/cm², and accumulated light dose: 600 mJ/cm²; measured with UIT-150 manufactured by USHIO INC.) to form a hydrophilic monolayer film having a film thickness of 3.5 μm thick, on the transparent acrylic plate. The surface of the monolayer film was then washed with running water, followed by drying, and the resulting sample was evaluated. The results are shown in Table 4-1.

### Example 2

The same operation as in Example 1 was carried out except that the diluting solvent in Example 1 was replaced by a mixed solvent of 41.3 g of methanol and 20.7 g of propylene glycol monomethyl ether (PGM). The thus prepared coating solution having a solid concentration of 50 wt % was coated on a transparent acrylic plate in the same manner as in Example 1, followed by solvent removal and UV irradiation, to form a monolayer film having a film thickness of 4 μm on the transparent acrylic plate. The surface of the monolayer film was then washed with running water, followed by drying, and the resulting sample was evaluated. The results are shown in Table 4-1.

### Example 3

The same operation as in Example 1 was carried out except that the diluting solvent in Example 1 was replaced by a mixed solvent of 55.8 g of ethanol and 6.2 g of distilled water. The thus prepared coating solution having a solid concentration of 50 wt % was coated on a transparent acrylic plate in the same manner as in Example 1, followed by solvent removal and UV irradiation, to form a monolayer film having a film thickness of 3.5 μm on the transparent acrylic plate. The surface of the monolayer film was then washed with running water, followed by drying, and the resulting sample was evaluated. The results are shown in Table 4-1.

### Example 4

### (Addition of Surfactant and Removal of Solvent)

Into an experimental apparatus shown in FIG. 2 that had been shielded from light with an aluminum foil, 125 g of the polymerizable composition 1 obtained in Preparation Example 1, and 1 g of the mixed solution of DS-Na-1 obtained in Preparation Example 4-1 and having a solid concentration of 10 wt % were charged. While bubbling the resultant with dry air (having a dew point of −30° C. or less), the solution was maintained at a reduced pressure (<100 mmHg) for 3 days (at room temperature) to remove the solvent. As a result, a slightly white, highly viscous polymerizable liquid was obtained. The highly viscous liquid was analyzed by GC (internal standard method), and the amount of the residual solvent (methanol) was determined to be <0.1 wt %. The GC conditions are described below.

### GC Analysis Conditions

GC device: Shimadzu Corporation, GC-2010

Column: J & W Science, DB-624, φ 0.53 mm×75 m (film thickness: 3 μm)

Carrier gas: He 100 cm/sec

Inj.: 240° C.

Det.: FID, 260° C.

### Sample Preparation

IS (internal standard): 2-methoxy-1-ethanol, 50 mg

Sample: polymerizable composition, 100 mg

Diluting solvent: dichloromethane, 10 ml

Injection volume: 1 μl

### (Preparation of Coating Solution)

To 10.0 g of the highly viscous polymerizable liquid (solvent content<0.1 wt %) obtained above, 0.3 g of DAROCUR 1173 (BASF) as a polymerization initiator was added. The resultant was mixed carefully with a stirring rod to give a coating solution having a solid concentration of 100 wt %.

### (Coating on Substrate and UV Irradiation)

The thus obtained coating solution was coated on a transparent acrylic plate (CLAREX-001; manufactured by Nitto Jushi Kogyo Co., Ltd.) with a bar coater #10, and the resultant was left to stand at room temperature (23° C., 27% RH) for 30 minutes, followed by UV irradiation (electrodeless discharge lamp, H valve 240 W/cm, intensity of illumination: 200 mW/cm², and accumulated light dose: 150 mJ/cm², measured with UIT-150 manufactured by USHIO INC.), to form a monolayer film composed of a crosslinked resin, having a hydrophilic surface and a film thickness of 14 μm, on the surface of the transparent acrylic plate. Finally, the film surface was washed with running water, and dried with an air gun to give a sample for evaluation. The results are shown in Table 4-1.

### Example 5

A quantity of 100 g of the polymerizable composition 2 obtained in Preparation Example 2 and having a solid concentration of 80 wt %, 0.8 g (0.1 wt % with respect to the total weight of the compound (I) and the compound (II)) of the solution of DS-Na-1 (the solution of the surfactant (III)) obtained in Preparation Example 4-1 and having a solid concentration of 10 wt %, a mixed solvent of 113.3 g of methanol and 56.7 g of PGM as a diluting solvent, and 2.4 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 30 wt %. A transparent acrylic plate (CLAREX-001; manufactured by Nitto Jushi Kogyo Co., Ltd.) pretreated in advance was immersed in the thus obtained solution, and pulled up at 1 mm/sec, to coat the solution on the substrate surface. The resultant was placed in a hot air dryer controlled at 50 to 60° C. for 5 minutes, to remove the solvent contained in the coating.

After sufficiently removing the solvent, the sample was subjected to UV irradiation by being passed through a UV conveyor (high pressure mercury lamp, 160 W/cm, height: 19 cm, conveyor speed: 5 m/min, intensity of illumination: 200 mW/cm², and accumulated light dose: 600 mJ/cm²; measured with UIT-150 manufactured by USHIO INC.) to form a hydrophilic monolayer film having a film thickness of 0.5 μm thick, on the transparent acrylic plate. The surface of the monolayer film was then washed with running water, followed by drying, and the resulting sample was evaluated. The results are shown in Table 4-1.

### Example 6

The same operation as in Example 5 was carried out except that the diluting solvent in Example 5 was replaced by a mixed solvent of 41.3 g of methanol and 20.7 g PGM. The thus prepared coating solution having a solid concentration of 50 wt % was coated on a transparent acrylic plate in the same manner as in Example 5, followed by solvent removal and UV irradiation, to form a monolayer film having a film thickness of 4 μm on the transparent acrylic plate. The surface of the monolayer film was then washed with running water, followed by drying, and the resulting sample was evaluated. The results are shown in Table 4-1.

### Example 7

The same operation as in Example 5 was carried out except that the diluting solvent in Example 5 was replaced by a mixed solvent of 23.3 g of methanol and 11.7 g PGM. The thus prepared coating solution having a solid concentration of 60 wt % was coated on a transparent acrylic plate in the same manner as in Example 5, followed by solvent removal and UV irradiation, to form a monolayer film having a film thickness of 7 μm on the transparent acrylic plate. The surface of the monolayer film was then washed with running water, followed by drying, and the resulting sample was evaluated. The results are shown in Table 4-1.

### Comparative Example 1

A transparent acrylic plate (CLAREX-001; manufactured by Nitto Jushi Kogyo Co., Ltd.) was washed with running water, followed by drying, and the resulting sample was evaluated. Comparative Example 1 corresponds to the case in which the transparent acrylic plate as a substrate was used as it is, without being subjected to either coating with the dental composition of the invention or curing.

The results are shown in Tables 4-1, 4-2, 5 and 7.

### Comparative Example 2

A transparent acrylic plate made of Acron (GC), which is used as a dental material, was prepared, and washed with running water, followed by drying, and the resulting sample was evaluated. Comparative Example 2 corresponds to the case in which a resin commercially available as a denture base material was used as it is, without being subjected to either coating with the dental composition of the invention or curing.

The results are shown in Tables 4-1, 4-2, 5 and 7.

TABLE 4-1

| | | | | | | Water | Color |
|---|---|---|---|---|---|---|---|
| | Basal | Diluting | Concentration | | | contact | difference |
| No. | Composition | solvent | (wt %) | Surfactant | Appearance | angle (°) | ΔE*ab |
| Example 1 | Composition | Methanol | 50 | DS-Na-1 | Transparent | 7 | 0.9 |
| | 1 | | | 0.1% | | | |
| Example 2 | Composition | Methanol/ | 50 | DS-Na-1 | Transparent | 6 | 1.1 |
| | 1 | PGM | | 0.1% | | | |
| Example 3 | Composition | Ethanol/ | 50 | DS-Na-1 | Transparent | 9 | 1.3 |
| | 1 | Water | | 0.1% | | | |
| Example 4 | Composition | None | 100 | DS-Na-1 | Transparent | 10 | 1.9 |
| | 1 | | | 0.1% | | | |
| Example 5 | Composition | Methanol/ | 30 | DS-Na-1 | Transparent | 11 | 0.6 |
| | 2 | PGM | | 0.1% | | | |
| Example 6 | Composition | Methanol/ | 50 | DS-Na-1 | Transparent | 7 | 0.5 |
| | 2 | PGM | | 0.1% | | | |
| Example 7 | Composition | Methanol/ | 60 | DS-Na-1 | Transparent | 8 | 0.6 |
| | 2 | PGM | | 0.1% | | | |
| Comparative | — | — | — | — | Transparent | 75 | 6.0 |
| Example 1 | | | | | | | |
| Comparative | — | — | — | — | Transparent | 80 | 6.6 |
| Example 2 | | | | | | | |

### Example 8

A quantity of 100 g of the polymerizable composition 3 obtained in Preparation Example 3 and having a solid concentration of 80 wt %, 0.8 g (0.1 wt % with respect to the total weight of the compound (I) and the compound (II)) of the solution of DS-Na-2 (the solution of the surfactant (III)) obtained in Preparation Example 4-2 and having a solid concentration of 10 wt %, a mixed solvent of 41.3 g of methanol and 20.7 g of PGM as a diluting solvent, and 2.4 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 50 wt %. A transparent acrylic plate (CLAREX-001; manufactured by Nitto Jushi Kogyo Co., Ltd.) pretreated in advance was immersed in the thus obtained solution, and pulled up at 1 mm/sec, to coat the solution on the substrate surface. The resultant was placed in a hot air dryer controlled at 50 to 60° C. for 5 minutes, to remove the solvent contained in the coating.

After sufficiently removing the solvent, the sample was subjected to UV irradiation by being passed through a UV conveyor (high pressure mercury lamp, 160 W/cm, height: 19 cm, conveyor speed: 5 m/min, intensity of illumination: 200 mW/cm², and accumulated light dose: 600 mJ/cm²; measured with UIT-150 manufactured by USHIO INC.) to form a hydrophilic monolayer film on the transparent acrylic plate. The surface of the monolayer film was then washed with running water, followed by drying, and the resulting sample was evaluated. The results are shown in Table 4-2.

### Example 9

A quantity of 100 g of the polymerizable composition 3 obtained in Preparation Example 3 and having a solid concentration of 80 wt %, 0.8 g (0.1 wt % with respect to the total weight of the compound (I) and the compound (II)) of the solution of DS-Na-2 (the solution of the surfactant (III)) obtained in Preparation Example 4-2 and having a solid concentration of 10 wt %, a mixed solvent of 55.8 g of ethanol and 6.2 g of distilled water as a diluting solvent, and 2.4 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 50 wt %. A transparent acrylic plate (CLAREX-001; manufactured by Nitto Jushi Kogyo Co., Ltd.) pretreated in advance was immersed in the thus obtained solution, and pulled up at 1 mm/sec, to coat the solution on the substrate surface. The resultant was placed in a hot air dryer controlled at 50 to 60° C. for 5 minutes, to remove the solvent contained in the coating.

After sufficiently removing the solvent, the sample was subjected to UV irradiation by being passed through a UV conveyor (high pressure mercury lamp, 160 W/cm, height: 19 cm, conveyor speed: 5 m/min, intensity of illumination: 200 mW/cm², and accumulated light dose: 600 mJ/cm²; measured with UIT-150 manufactured by USHIO INC.) to form a hydrophilic monolayer film on the transparent acrylic plate. The surface of the monolayer film was then washed with running water, followed by drying, and the resulting sample was evaluated. The results are shown in Table 4-2.

### Example 10

The same operation as in Example 9 was carried out except that the solution of the surfactant (III) in Example 9 was replaced by the solution of DT-Na obtained in Preparation Example 4-3, having a solid concentration of 10 wt %. The thus prepared coating solution having a solid concentration of 50 wt % was coated on a transparent acrylic plate in the same manner as in Example 9, followed by solvent removal and UV irradiation, to form a monolayer film. The surface of the monolayer film was then washed with running water, followed by drying, and the resulting sample was evaluated. The results are shown in Table 4-2.

### Example 11

The same operation as in Example 9 was carried out except that the solution of the surfactant (III) in Example 9 was replaced by the solution of DH-NH4 obtained in Preparation Example 4-4 and having a solid concentration of 10 wt %. The thus prepared coating solution having a solid concentration of 50 wt % was coated on a transparent acrylic plate in the same manner as in Example 9, followed by solvent removal and UV irradiation, to form a monolayer film. The surface of the monolayer film was then washed with running water, followed by drying, and the resulting sample was evaluated. The results are shown in Table 4-2.

### Example 12

The same operation as in Example 9 was carried out except that the solution of the surfactant (III) in Example 9 was replaced by the solution of LS-Na obtained in Preparation Example 4-5 and having a solid concentration of 10 wt %. The thus prepared coating solution having a solid concentration of 50 wt % was coated on a transparent acrylic plate in the same manner as in Example 9, followed by solvent removal and UV irradiation, to form a monolayer film. The surface of the monolayer film was then washed with running water, followed by drying, and the resulting sample was evaluated. The results are shown in Table 4-2.

TABLE 4-2

| | | | | | | Water | Color |
|---|---|---|---|---|---|---|---|
| | Basal | Diluting | Concentration | | | contact | difference |
| No. | Composition | solvent | (wt %) | Surfactant | Appearance | angle (°) | ΔE*ab |
| Example 8 | Composition | Methanol/ | 50 | DS-Na-2 | Transparent | 8 | 0.7 |
| | 3 | PGM | | 0.1% | | | |
| Example 9 | Composition | Ethanol/ | 50 | DS-Na-2 | Transparent | 7 | 0.8 |
| | 3 | Water | | 0.1% | | | |
| Example 10 | Composition | Ethanol/ | 50 | DT-Na | Transparent | 8 | 1.1 |
| | 3 | Water | | 0.1% | | | |
| Example 11 | Composition | Ethanol/ | 50 | DH-NH4 | Transparent | 10 | 1.5 |
| | 3 | Water | | 0.1% | | | |
| Example 12 | Composition | Ethanol/ | 50 | LS-Na | Transparent | 17 | 2.0 |
| | 3 | Water | | 0.1% | | | |
| Comparative | — | — | — | — | Transparent | 75 | 6.0 |
| Example 1 | | | | | | | |
| Comparative | — | — | — | — | Transparent | 80 | 6.6 |
| Example 2 | | | | | | | |

### (Analysis of Gradient of Anion Concentration in Direction of Film Thickness of Monolayer Film)

For each of the monolayer films obtained in Examples 1 to 9, the concentration of anions (Sa) at the top surface and the concentration of anions (Da) at the midpoint in the direction of the film thickness (the midpoint between the top surface and the bottom surface of the film) were measured. The gradient (Sa/Da) of the concentration of anions of each of the monolayer films, obtained based on the thus measured values, was 1.1 or more.

### Preparation Example 5

### (Preparation of Solution of Compound (I): ATBS-Na)

To 10 g of sodium 2-acrylamido-2-methylsulfonate (hereinafter, abbreviated as ATBS-Na) obtained by neutralizing 2-acrylamido-2-methylsulfonic acid (hereinafter, abbreviated as ATBS) with sodium hydroxide and then by drying, 30 g of water was added. The resultant was ultrasonically mixed to dissolve the components. Then 60 g of 1-methoxy-2-propanol (hereinafter, abbreviated as PGM) was added, and the resultant was vigorously stirred to give a mixed solution of ATBS-Na having a solid concentration of 10 wt %.

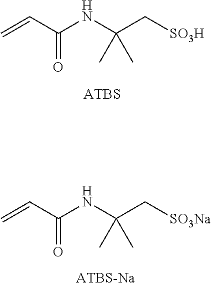

### Example 13

### (Preparation of Coating Solution)

A quantity of 50 g of the mixed solution of ATBS-Na having a solid concentration of 10 wt % (Preparation Example 5) as the compound (I), 100 g of dipentaerythritol pentaacrylate (hereinafter, abbreviated as A-9530) as the compound (II), 1.1 g of the mixed solution of DS-Na-1 having a solid concentration of 10 wt % (Preparation Example 4-1) as the surfactant (III), 3 g of DAROCUR 1173 as a polymerization initiator, and 62 g of 2-methoxy-1-ethanol (hereinafter, abbreviated as EGM) as a diluting solvent were mixed, and the components were dissolved to give a coating solution having a solid concentration of 50 wt %. A transparent acrylic plate (CLAREX-001; manufactured by Nitto Jushi Kogyo Co., Ltd.) pretreated in advance was immersed in the thus obtained solution, and pulled up at 1 mm/sec, to coat the solution on the substrate surface. The resultant was placed in a hot air dryer controlled at 50 to 60° C. for 5 minutes, to remove the solvent contained in the coating.

After sufficiently removing the solvent, the sample was subjected to UV irradiation by being passed through a UV conveyor (high pressure mercury lamp, 160 W/cm, height: 19 cm, conveyor speed: 5 m/min, intensity of illumination: 200 mW/cm², and accumulated light dose: 600 mJ/cm²; measured with UIT-150 manufactured by USHIO INC.) to form a hydrophilic monolayer film on the transparent acrylic plate. The surface of the monolayer film was then washed with running water, followed by drying, and the resulting sample was evaluated.

The results are shown in Table 5.

### Example 14

The same operation as in Example 13 was carried out except that the solution of the surfactant (III) and the diluting solvent in Example 13 were replaced by 2.2 g of the mixed solution of DS-Na-1 having a solid concentration of 10 wt % (Preparation Example 4-1) and 61 g of EGM, respectively. The thus prepared coating solution having a solid concentration of 50 wt % was coated on a transparent acrylic plate in the same manner as in Example 13, followed by solvent removal and UV irradiation, to form a monolayer film on the transparent acrylic plate. The surface of the monolayer film was then washed with running water, followed by drying, and the resulting sample was evaluated. The results are shown in Table 5.

### Example 15

The same operation as in Example 13 was carried out, except that the solution of the surfactant (III) and the diluting solvent in Example 13 were replaced by 5.5 g of the mixed solution of DS-Na-1 having a solid concentration of 10 wt % (Preparation Example 4-1) and 58 g of EGM, respectively. The thus prepared coating solution having a solid concentration of 50 wt % was coated on a transparent acrylic plate in the same manner as in Example 13, followed by solvent removal and UV irradiation, to form a monolayer film on the transparent acrylic plate. The surface of the monolayer film was then washed with running water, followed by drying, and the resulting sample was evaluated. The results are shown in Table 5.

### Example 16

The same operation as in Example 13 was carried out, except that the solution of the surfactant (III) and the diluting solvent in Example 13 were replaced by 11 g of the mixed solution of DS-Na-1 having a solid concentration of 10 wt % (Preparation Example 4-1) and 54 g of EGM, respectively. The thus prepared coating solution having a solid concentration of 50 wt % was coated on a transparent acrylic plate in the same manner as in Example 13, followed by solvent removal and UV irradiation, to form a monolayer film on the transparent acrylic plate. The surface of the monolayer film was then washed with running water, followed by drying, and the resulting sample was evaluated. The results are shown in Table 5.

TABLE 5

| | | | | | Water | Color |
|---|---|---|---|---|---|---|
| | Compound | Compound | surfactant | | contact | difference |
| No. | (I) | (II) | (III) | Appearance | angle (°) | ΔE*ab |
| Example 13 | ATBS-Na | A-9530 | DS-Na-1 | Slightly | 21 | 2.0 |
| | | | 0.1% | white | | |
| Example 14 | ATBS-Na | A-9530 | DS-Na-1 | Transparent | 18 | 1.5 |
| | | | 0.2% | | | |
| Example 15 | ATBS-Na | A-9530 | DS-Na-1 | Slightly | 20 | 2.2 |
| | | | 0.5% | white | | |
| Example 16 | ATBS-Na | A-9530 | DS-Na-1 | Slightly | 25 | 2.5 |
| | | | 1.0% | white | | |
| Comparative | — | — | — | Transparent | 75 | 6.0 |
| Example 1 | | | | | | |
| Comparative | — | — | — | Transparent | 80 | 6.6 |
| Example 2 | | | | | | |

### Examples 17 to 19, 21, and Reference Example 20

### (Preparation of Polymerizable Composition)

Polymerizable compositions 5A to 5E were prepared in accordance with the respective formulations shown in Table 6 below. The symbols in Table 6 indicate compounds represented by the formulas shown below.

TABLE 6
Composition of polymerizable compositions 5A to 5E

| | | | | | | | Polymerizable | | | | |
|---|---|---|---|---|---|---|---|---|---|---|---|
| | Polymerizable | | Polymerizable | | Polymerizable | | composition 5D | | Polymerizable | | |
| | composition 5A | | composition 5B | | composition 5C | | (Reference | | composition 5E | | |
| | (Example 17) | | (Example 18) | | (Example 19) | | Example 20) | | (Example 21) | | |
| | | Concen- | | Concen- | | Concen- | | Concen- | | Concen- | |
| | Amount | tration | Amount | tration | Amount | tration | Amount | tration | Amount | tration | |
| Materials | (g) | (wt %) | (g) | (wt %) | (g) | (wt %) | (g) | (wt %) | (g) | (wt %) | Note |
| VS-Na | 5.00 | 2.35 | | | | | | | | | Compound |
| | | | | | | | | | | | (I) |
| STS-Na | | | 5.00 | 2.35 | | | | | | | |
| P1A-Na | | | | | 5.00 | 2.34 | | | | | |
| HEA | | | | | | | 5.00 | 2.34 | | | |
| DMA-HCI | | | | | | | | | 5.00 | 2.34 | |
| A-9530 | 100.00 | 46.90 | | | 100.00 | 46.88 | 100.00 | 46.88 | 100.00 | 46.84 | Compound |
| | | | | | | | | | | | (II) |
| EA-5721 | | | 100.00 | 46.90 | | | | | | | |
| DS-Na | 0.22 | 0.10 | 0.22 | 0.10 | | | | | | | surfactant |
| | | | | | | | | | | | (III) |
| LB | | | | | 0.33 | 0.15 | 0.33 | 0.15 | | | |
| SSE | | | | | | | | | 0.55 | 0.26 | |
| EGM | 61.00 | 28.61 | 61.00 | 28.61 | 60.00 | 28.13 | 60.00 | 28.13 | 58.00 | 27.17 | Solvent |
| PGM | 31.32 | 14.69 | 31.32 | 14.69 | 31.98 | 14.99 | 31.98 | 14.99 | 33.30 | 15.60 | |
| Water | 15.66 | 7.35 | 15.66 | 7.35 | 15.99 | 7.50 | 15.99 | 7.50 | 16.65 | 7.80 | |
| Total | 213.20 | 100.00 | 213.20 | 100.00 | 213.30 | 100.00 | 213.30 | 100.00 | 213.50 | 100.00 | |
| Solid con- | | 49.35 | | 49.35 | | 49.38 | | 49.38 | | 49.44 | |
| centration | | | | | | | | | | | |
| (%) | | | | | | | | | | | |

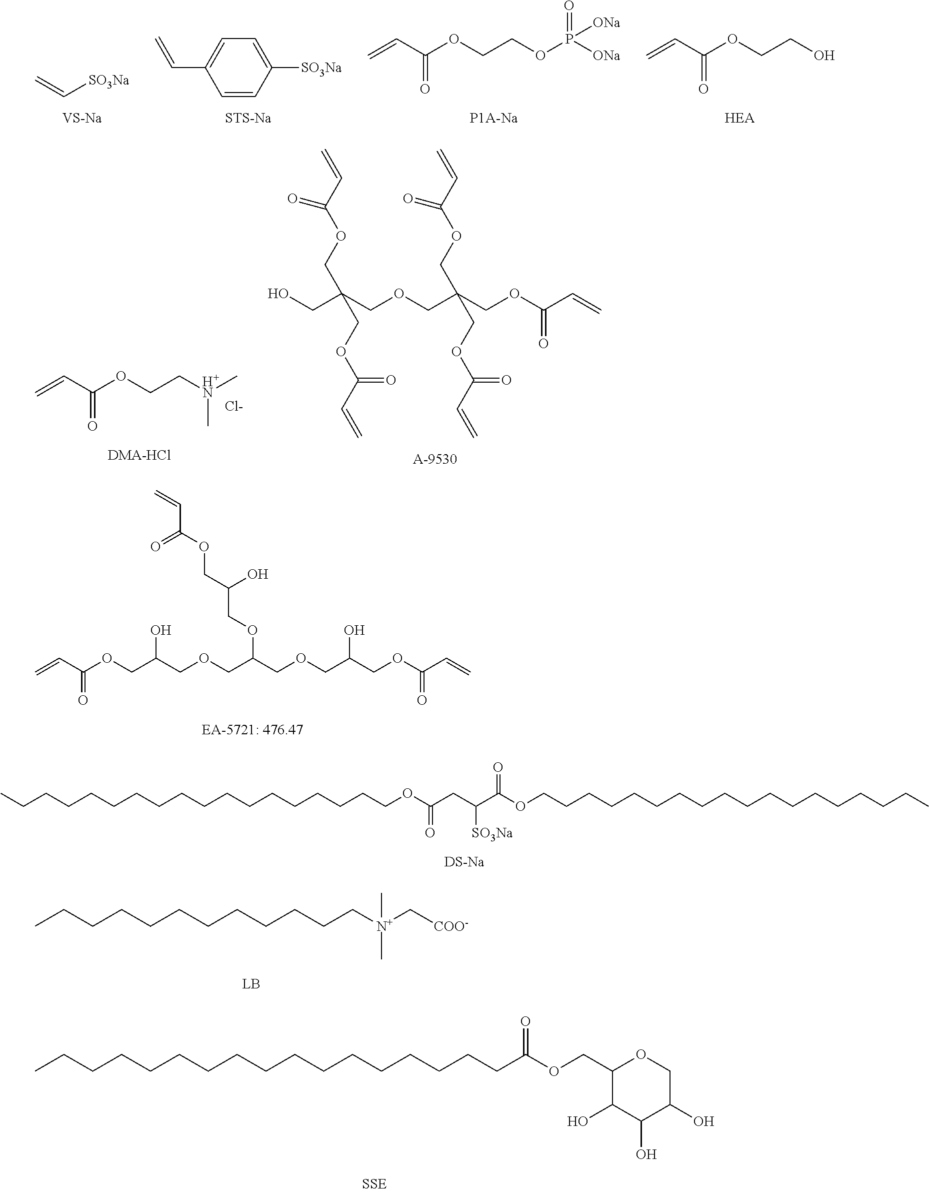

### Example 17

### (Coating on Substrate and Evaluation)

To the polymerizable composition 5A, 3 g of DAROCUR 1173 as a polymerization initiator was added to give a coating solution having a solid concentration of 50 wt %. A transparent acrylic plate (CLAREX-001; manufactured by Nitto Jushi Kogyo Co., Ltd.) pretreated in advance was immersed in the thus obtained solution, and pulled up at 1 mm/sec, to coat the solution on the substrate surface. The resultant was placed in a hot air dryer controlled at 50 to 60° C. for 5 minutes, to remove the solvent contained in the coating.

After sufficiently removing the solvent, the sample was subjected to UV irradiation by being passed through a UV conveyor (high pressure mercury lamp, 160 W/cm, height: 19 cm, conveyor speed: 5 m/min, intensity of illumination: 200 mW/cm², and accumulated light dose: 600 mJ/cm²; measured with UIT-150 manufactured by USHIO INC.) to form a hydrophilic monolayer film on the transparent acrylic plate. The surface of the monolayer film was then washed with running water, followed by drying, and the resulting sample was evaluated. The results are shown in Table 7.

### Examples 18, 19, 21, and Reference Example 20

In each of the Examples, the same operation as in Example 17 was carried out, except that the polymerizable composition 5A in Example 17 was replaced by each of polymerizable compositions 5B to 5E. The results are shown in Table 7.

TABLE 7

| | | | | | Water | |
|---|---|---|---|---|---|---|
| | | | | | contact | Color |
| | Compound | Compound | surfactant | | angle | difference |
| No. | (I) | (II) | (III) | Appearance | (°) | ΔE*ab |
| Example 17 | VS-Na | A-9530 | DS-Na-1 | White | 15 | 1.8 |
| | | | 0.2% | | | |
| Example 18 | STS-Na | EA-5721 | DS-Na-1 | Transparent | 28 | 2.5 |
| | | | 0.2% | | | |
| Example 19 | P1A-Na | A-9530 | LB | Slightly | 10 | 1.7 |
| | | | 0.3% | white | | |
| Reference | HEA | A-9530 | LB | Transparent | 40 | 2.8 |
| Example 20 | | | 0.3% | | | |
| Example 21 | DMA-HCL | A-9530 | SSE | Transparent | 30 | 2.9 |
| | | | 0.5% | | | |
| Comparative | — | — | — | Transparent | 75 | 6.0 |
| Example 1 | | | | | | |
| Comparative | — | — | — | Transparent | 80 | 6.6 |
| Example 2 | | | | | | |

### <Preparation of Dentures>

### (Preparation of Dentures Made of Wax)

Preliminary impressions of the upper jaw and lower jaw of a patient were taken, and a tray suited to the patient was prepared based on the preliminary impressions. The resulting tray was used to take precise impressions of the patient's jaws. Based on the thus taken precise impressions, upper and lower plaster figures suited to the patient were separately prepared.

Then the upper and lower plaster figures were connected, and bite plates each composed of a base plate and wax were prepared in order to reproduce the occlusion between the upper and lower jaws.

The oral cavity of the patient was then examined to observe the movement of jaws, and the jaw movement was reproduced with the above prepared bite plates to obtain the state of occlusion of the patient in three dimensions, and thus the position of occlusion was determined. Based on the position, denture bases (a pair of a denture base for upper jaw and a denture base for lower jaw) made of wax were prepared.

Onto the resulting denture bases made of wax, artificial teeth were arranged, followed by try-in and adjustment, thereby completing dentures (a pair of a denture for upper jaw and a denture for lower jaw) made of wax.

### (Preparation of Plaster Molds for Dentures)

First, a flask for preparation of dentures consisting of a lower mold flask and an upper mold flask was prepared.

Then the denture made of wax and the plaster figure were combined and placed in the lower mold flask, and dental plaster mixed with a specified amount of water was fully filled into the lower mold flask. The resultant was then allowed to stand for a while. After the plaster had solidified, a separating agent was dropped on the plaster, and coated over the entire surface of the plaster with a brush. Subsequently, the upper mold flask was placed on the lower mold flask, and the plaster was filled therein to the limit. A lid was placed over it and the resultant was allowed to stand until the plaster was completely solidified.

After the plaster had solidified, the upper mold flask and the lower mold flask were separated, warmed with hot water to allow wax to melt, and the base plate was removed.

Thus, plaster molds for dentures consisting of a pair of an upper plaster mold and a lower plaster mold were prepared.

Then the separating agent was coated over the entire surfaces of the upper plaster mold and the lower plaster mold.

### (Preparation of Dentures)

The flask for preparation of dentures with which the plaster molds for dentures were prepared was used, and MMA was allowed to polymerize inside the plaster molds to give dentures made of PMMA, followed by polishing. Detailed operation will be described below.

First, a resin material for denture base, Acron Clear No. 5 (manufactured by GC Corporation) was prepared, and 12 g of the powder and 5 g of the liquid were weighed and introduced into a container, followed by mixing. The resulting mixture was left to stand for some time, and when the mixture had turned into a sticky paste-like state, a more than sufficient amount of the sticky paste-like mixture was placed on the cavities on the lower plaster mold prepared inside the lower mold flask, and then the shape of the mixture was fixed up.

Next, onto the lower mold flask, the upper mold flask inside which the upper plaster mold was prepared was placed, and a pressure was applied using a pressing machine. The upper mold flask was once removed, and the sticky paste-like resin material for denture base protruding out of the cavities was removed. Then the upper mold flask was placed back on the lower mold flask again, and a pressure was applied using a pressing machine. Subsequently, a flask clamp was used to fix the flask (the flask consisting of the upper mold flask and the lower mold flask combined).

The flask was placed in a pan containing water, and slowly heated on a gas range to 100° C. over 30 minutes or more. When the temperature had reached 100° C., the flask was heated for another to 40 minutes, and then the heating was terminated, followed by cooling to 30° C.

Then, the lower mold flask and the upper mold flask were separated, followed by cracking the plaster molds. Completed dentures (made of PNMA) were retrieved and subjected to polishing finish.

### <Pretreatment of Dentures>

The polished dentures were immersed in a mixed solution of acetone and IPA (isopropyl alcohol) (at a weight ratio of 1:1) for 5 minutes, retrieved from the solution, and then subjected to air blowing. Then the dentures were dried in a fan dryer controlled at 40° C. for 5 minutes, to be used for the coating.

### Example 22

A quantity of 300 g of the polymerizable composition 1 obtained in Preparation Example 1 and having a solid concentration of 80 wt %, 2.4 g (0.1 wt % with respect to the total weight of the compound (I) and the compound (II)) of the solution of DS-Na-1 (the solution of the surfactant (III)) obtained in Preparation Example 4-1 and having a solid concentration of 10 wt %, 186 g of methanol as a diluting solvent, and 7.2 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 50 wt %, which is the same as that obtained in Example 1. The dentures pretreated in advance as described in the section of “Pretreatment of dentures” above were immersed in the thus obtained solution, and pulled up at 1 mm/sec, to coat the solution on the surface of the dentures. The resulting dentures were placed in a hot air dryer controlled at 50 to 60° C. for 5 minutes, to remove the solvent contained in the coating.

After sufficiently removing the solvent, the sample was subjected to UV irradiation by passing it through a UV conveyor (high pressure mercury lamp, 160 W/cm, height: 19 cm, conveyor speed: 5 m/min, intensity of illumination: 200 mW/cm², and accumulated light dose: 600 mJ/cm²; measured with UIT-150 manufactured by OSHIO INC.), and then by turning the sample over and passing it through the conveyor again, to form a hydrophilic monolayer film on the surface of each of the dentures.

### Example 23

A quantity of 300 g of the polymerizable composition 2 obtained in Preparation Example 2 and having a solid concentration of 80 wt %, 2.4 g (0.1 wt % with respect to the total weight of the compound (I) and the compound (II)) of the solution of DS-Na-1 (the solution of the surfactant (III)) obtained in Preparation Example 4-1 and having a solid concentration of 10 wt %, a mixed solvent of 123.9 g of methanol and 62.1 g of PGM as a diluting solvent, and 7.2 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 50 wt %, which is the same as that obtained in Example 6. Then, dip coating, drying, and UV irradiation were carried out in the same manner as in Example 22, to form a hydrophilic monolayer film on the surface of each of the dentures.

### Example 24

A quantity of 300 g of the polymerizable composition 3 obtained in Preparation Example 3 and having a solid concentration of 80 wt %, 2.4 g (0.1 wt % with respect to the total weight of the compound (I) and the compound (II)) of the solution of DS-Na-2 (the solution of the surfactant (III)) obtained in Preparation Example 4-2 and having a solid concentration of 10 wt %, a mixed solvent of 167.4 g of ethanol and 18.6 g of distilled water as a diluting solvent, and 7.2 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 50 wt %, which is the same as that obtained in Example 9. Then, dip coating, drying, and UV irradiation were carried out in the same manner as in Example 22, to form a hydrophilic monolayer film on the surface of each of the dentures.

### Comparative Example 3

The dentures obtained in the same manner as described above were subjected to the following evaluations, without performing the coating with the dental composition of the invention.

### <Evaluation of Contamination Resistance 1>

The coated dentures prepared in Examples 22 to 24 and the non-coated dentures prepared in Comparative Example 3 were each washed with water and dried. Then a mark was made on each of the dentures using a permanent marker, “Mackey ultrafine” manufactured by Zebra co., Ltd., (black, model number: MO-120-MC-BK), followed by washing with running water. Most of the marks made on the coated dentures prepared in Examples 22 to 24 were washed away with water, or able to be removed with a light rub. On the other hand, the marks made on the uncoated dentures prepared in Comparative Example 3 did not come off at all, even with rubbing.

### <Evaluation of Contamination Resistance 2>

The coated dentures prepared in Examples 22 to 24 and the non-coated dentures prepared in Comparative Example 3 were immersed in a lipophilic colorant (Bon Curry Gold, medium hot (solid ingredients were removed); manufactured by Otsuka Foods Co., Ltd.) and maintained in that state at 40° C. for 6 hours. After washing the test specimens with running water, the test specimens were immersed in distilled water, and maintained at that state at room temperature for 12 to 18 hours. The above described operation was repeated 6 times, and after the 7th washing with running water, the degree of fouling was visually observed.

No oil stain or coloration was observed on the coated dentures prepared in Examples 22 to 24. On the other hand, the uncoated dentures prepared in Comparative Example 3 had oil stains on the surfaces and between the teeth thereof.

### <Preparation of Dentures 2>

### (Preparation of Dentures Made of Wax)

Preliminary impressions of the upper jaw and lower jaw of a patient were taken, and a tray suited to the patient was prepared based on the preliminary impressions. The resulting tray was used to take precise impressions of the patient's jaws. Based on the thus taken precise impressions, upper and lower plaster figures suited to the patient were separately prepared.

Then the upper and lower plaster figures were connected, and bite plates each composed of a base plate and wax were prepared in order to reproduce the occlusion between the upper and lower jaws.

The oral cavity of the patient was then examined to observe the movement of jaws, and the jaw movement was reproduced with the above prepared bite plates to obtain the state of occlusion of the patient in three dimensions, and thus the position of occlusion was determined. Based on the position, denture bases (a pair of a denture base for upper jaw and a denture base for lower jaw) made of wax were prepared.

Onto the resulting denture bases made of wax, artificial teeth (e-Ha; manufactured by Heraeus Kulzer GmbH.) coated with a wax pattern separating agent, SEP (manufactured by SHOFU Inc.) in advance were arranged, followed by try-in and adjustment, thereby completing dentures (a pair of a denture for upper jaw and a denture for lower jaw) made of wax.

### (Preparation of Plaster Molds for Denture Bases)

First, a flask for preparation of dentures consisting of an upper mold flask and a lower mold flask was prepared.

Further, the artificial teeth were removed from the dentures made of wax, to prepare denture bases made of wax.

Then the denture base made of wax and the plaster figure were combined and placed in the lower mold flask, and dental plaster mixed with a specified amount of water was fully filled into the lower mold flask. The resultant was then allowed to stand for a while. After the plaster had solidified, the above mentioned separating agent was dropped on the plaster, and coated over the entire surface of the plaster with a brush. Subsequently, the upper mold flask was placed on the lower mold flask, and the plaster was filled therein to the limit. A lid was placed over it and the resultant was allowed to stand until the plaster was completely solidified.

After the plaster had solidified, the upper mold flask and the lower mold flask were separated, warmed with hot water to allow wax to melt, and the base plate was removed.

Thus, plaster molds for denture bases, consisting of a pair of an upper plaster mold and a lower plaster mold, were prepared.

At this time, the upper plaster mold was prepared inside the upper mold flask and the lower plaster mold was prepared inside the lower mold flask. The plaster molds were prepared such that, when the upper plaster mold and the lower plaster mold were combined, a space having the shape of the combined denture bases made of wax is formed inside the plaster molds.

Then the separating agent was coated over the entire surfaces of the upper plaster mold and the lower plaster mold.

### (Preparation of Conventional Denture Bases)

The flask for preparation of dentures with which the plaster molds for denture bases were prepared was used, and MMA was allowed to polymerize inside the plaster molds to give conventional dentures (a pair of an upper jaw denture base and lower jaw denture base; both made of PMMA). Detailed operation will be described below.

First, a resin material for denture base, Acron Clear No. 5 (manufactured by GC Corporation) was prepared, and 6 parts by weight of the powder and 2.5 parts by weight of the same in the liquid were weighed and introduced into a container, followed by mixing. The resulting mixture was left to stand for some time, and when the mixture had turned into a sticky paste-like state, a more than sufficient amount of the sticky paste-like mixture was placed on the cavities on the lower plaster mold prepared inside the lower mold flask, and then the shape of the mixture was fixed up.

Next, onto the lower mold flask, the upper mold flask inside which the upper plaster mold was prepared was placed, and a pressure was applied using a pressing machine. The upper mold flask was once removed, and the sticky paste-like resin material for denture base protruding out of the cavities was removed. Then the upper mold flask was placed back on the lower mold flask again, and a pressure was applied using a pressing machine. Subsequently, a flask clamp was used to fix the flask (the flask consisting of the upper mold flask and the lower mold flask combined).

The flask was placed in a pan containing water, and slowly heated on a gas range to 100° C. over 30 minutes or more. When the temperature had reached 100° C., the flask was heated for another to 40 minutes, and then the heating was terminated, followed by cooling to 30° C.

Then, the lower mold flask and the upper mold flask were separated, followed by cracking the plaster molds. Completed denture bases (made of PMMA) were retrieved and subjected to polishing to give conventional denture bases.

### (Preparation of CAD/CAM Denture Bases)

The 3D data of the conventional denture bases prepared above were obtained using a 3D scanner.

Using CAD/CAM software, a cutting program for cutting a PMA resin block (CL-000; manufactured by Nitto Jushi Kogyo Co., Ltd.), as a denture base material, to obtain the denture bases was designed, based on the thus obtained 3D data.

According to the thus designed cutting program, the resin block was cut using a CNC cutting machine, to give CAD/CAM denture bases.

### (Preparation of CAD/CAM Dentures)

To all the sockets on the CAD/CAM denture bases prepared above, artificial teeth (e-Ha; manufactured by Heraeus Kulzer GmbH.) were bonded using a dental acrylic resin (Metafast; manufactured by Sun Medical Co., Ltd.) as an adhesive, to give CAD/CAM dentures.

### <Pretreatment of Dentures 2>

The CAD/CAM dentures prepared as described in the section of “Preparation of dentures 2” were polished, and then immersed in IPA (isopropyl alcohol) for 5 minutes. Then the dentures were retrieved from the IPA, and subjected to air blowing. The dentures were then dried in a fan dryer controlled at 40° C. for 5 minutes, and the dried dentures were used for the coating.

### Example 25

A quantity of 300 g of the polymerizable composition 3 obtained in Preparation Example 3 and having a solid concentration of 80 wt %, 2.4 g (0.1 wt % with respect to the total weight of the compound (I) and the compound (II)) of the solution of DS-Na-2 (the solution of the surfactant (III)) obtained in Preparation Example 4-2 and having a solid concentration of 10 wt %, a mixed solvent of 149.5 g of ethanol and 16.2 g of distilled water as a diluting solvent, and 7.2 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 52 wt %.

The dentures pretreated in advance as described in the section of “Pretreatment of dentures 2” above were immersed in the thus obtained solution, and pulled up at 1 mm/sec, to coat the solution on the surfaces of the dentures. The resulting dentures were then placed in a hot air dryer controlled at 50 to 60° C. for 5 minutes, to remove the solvent contained in the coating. The dentures after drying were subjected to UV irradiation by passing them through a UV conveyor (high pressure mercury lamp, 160 W/cm, height: 14 cm, conveyor speed: 5 m/min, intensity of illumination: 400 mW/cm², and accumulated light dose: 1200 mJ/cm²; measured with UIT-250 manufactured by USHIO INC.), and then by turning the dentures over and passing them through the conveyor again so that the entire surfaces thereof were irradiated, to form a hydrophilic monolayer film on the surface of each of the dentures.

### Examples 26 to 30

The same operation as in Example 25 was carried out, except that the type of the solution of the surfactant (III) and the amount of ethanol and distilled water in the diluting solvent in Example were changed to those shown in Table 8, to give coating solutions each having a solid concentration shown in Table 8. In each of the Examples, the thus prepared coating solution was coated on the surfaces of the dentures in the same manner as in Example 25, followed by solvent removal and UV irradiation, to form a monolayer film on the surface of each the dentures.

In Table 8, “DT-Na” in the section of the solution of the surfactant (III) indicates that the solution of DT-Na obtained in Preparation Example 4-3 and having a solid concentration of 10 wt % was used as the solution of the surfactant (III).

TABLE 8
Composition of coating solutions prepared in Examples 25 to 30

| | | Example 25 | Example 26 | Example 27 | Example 28 | Example 29 | Example 30 |
|---|---|---|---|---|---|---|---|
| Polymerizable | Type | Polymerizable | Polymerizable | Polymerizable | Polymerizable | Polymerizable | Polymerizable |
| composition | | composition 3 | composition 3 | composition 3 | composition 3 | composition 3 | composition 3 |
| | Amount (g) | 300 | 300 | 300 | 300 | 300 | 300 |
| | Solid concentration | 80 | 80 | 80 | 80 | 80 | 80 |
| | (wt %) | | | | | | |
| | Total Concentration | 79.85 | 79.85 | 79.85 | 79.85 | 79.85 | 79.85 |
| | of [Compound (I) + | | | | | | |
| | Compound (II)] | | | | | | |
| | (wt %) | | | | | | |
| Solution of | Type | DS-Na-2 | DS-Na-2 | DS-Na-2 | DT-Na | DT-Na | DT-Na |
| surfactant (III) | Amount (g) | 2.4 | 2.4 | 2.4 | 2.4 | 2.4 | 2.4 |
| | Solid concentration | 10 | 10 | 10 | 10 | 10 | 10 |
| | (wt %) | | | | | | |
| | Ratio with respect | 0.10 | 0.10 | 0.10 | 0.10 | 0.10 | 0.10 |
| | to [Compound (I) + | | | | | | |
| | Compound (II)] | | | | | | |
| | (wt %) | | | | | | |
| Photo- | Type | DAROCUR | DAROCUR | DAROCUR | DAROCUR | DAROCUR | DAROCUR |
| polymerization | | 1173 | 1173 | 1173 | 1173 | 1173 | 1173 |
| initiator | Amount (g) | 7.2 | 7.2 | 7.2 | 7.2 | 7.2 | 7.2 |
| | Ratio with respect | 3.0 | 3.0 | 3.0 | 3.0 | 3.0 | 3.0 |
| | to [Compound (I) + | | | | | | |
| | Compound (II)] | | | | | | |
| | (wt%) | | | | | | |
| Solvent | Ethanol (g) | 149.5 | 205.3 | 277.8 | 149.5 | 205.3 | 277.8 |
| | Distilled water (g) | 16.2 | 22.4 | 30.4 | 16.2 | 22.4 | 30.4 |
| Coating | Total Amount (g) | 475.3 | 537.3 | 617.8 | 475.3 | 537.3 | 617.8 |
| solutions | Total amount of | 247.44 | 247.44 | 247.44 | 247.44 | 247.44 | 247.44 |
| | solids (g) | | | | | | |
| | Solid concen- | 52 | 46 | 40 | 52 | 46 | 40 |
| | tration (wt %) | | | | | | |

### <Evaluation of Contamination Resistance 3>

The coated dentures prepared in Examples 25 to 30 were washed with water and dried. Then a mark was made on each of the dentures using a permanent marker, “Mackey ultrafine” manufactured by Zebra co., Ltd., (black, model number: MO-120-MC-BK), followed by washing with running water. Most of the marks made on the coated dentures were washed away with water, or able to be removed with a light rub.

### Visible Light Curing

### Example 31

A quantity of 1.26 g of the polymerizable composition 3 obtained in Preparation Example 3 and having a solid concentration of 80 wt %, 0.010 g of the solution of DS-Na-2 (the solution of the surfactant (III)) obtained in Preparation Example 4-2 and having a solid concentration of 10 wt %, a mixed solvent of 0.85 g of ethanol and 0.096 g of distilled water as a diluting solvent, and 0.022 g (1.0 wt % with respect to the total weight) of camphorquinone (manufactured by Wako Pure Chemical Industries, Ltd.) as a photopolymerization initiator were mixed, to give a coating solution having a solid concentration of 46 wt %.

A transparent acrylic plate (CLAREX-001; manufactured by Nitto Jushi Kogyo Co., Ltd.) pretreated in advance in the same manner as described in the section of the “Pretreatment of substrate” was used as a substrate, and the coating solution obtained above was coated on the surface of the substrate using a bar coater #30. The resultant was placed in a hot air dryer controlled at 50 to 60° C. for 5 minutes, to remove the solvent contained in the coating.

After sufficiently removing the solvent, the sample was placed in a visible light irradiation apparatus, Alpha light V (manufactured by MORITA Corporation, LED lamps; 400 to 408 nm, 465 to 475 nm: intensity of illumination: 60 mW/cm², accumulated light dose: 3600 mJ/cm², measured with UIT-250 (405 nm) manufactured by USHIO INC.), and irradiation was carried out for 1 minute, to form a hydrophilic monolayer film having a thickness of 18 μm on the transparent acrylic plate. The surface of the monolayer film was then washed with running water, followed by drying, and the appearance and the water contact angle of the resulting sample were evaluated. The results are shown in Table 9-1.

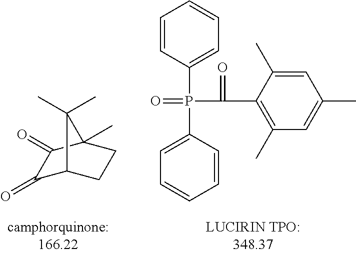

### Examples 32 to 42

The same operation as in Example 31 was carried out, except that the amount of the polymerizable composition 3, the type of the solution of the surfactant (III), and the amount and the type of the polymerization initiator in Example 31 were changed to those shown in Tables 9-1 to 9-2, to give coating solutions each having a solid concentration shown in Tables 9-1 to 9-2. In each of Examples, the thus prepared coating solution was coated on a transparent acrylic plate in the same manner as in Example 31, followed by solvent removal and visible light irradiation, to form a monolayer film having a film thickness of 18 μm on the transparent acrylic plate. The surface of each of the monolayer films was then washed with running water, followed by drying, and the appearance and the water contact angle of each of the resulting samples were evaluated. The results are shown in Tables 9-1 to 9-2.

In Table 9-2, “DT-Na” in the section of the solution of the surfactant (III) indicates that the solution of DT-Na obtained in Preparation Example 4-3 and having a solid concentration of 10 wt % was used as the solution of the surfactant (III).

Further, in Tables 9-1 and 9-2, “LUCIRIN TPO” in the section of the polymerization initiator indicates that LUCIRIN TPO (manufactured by BASF JAPAN LTD.) was used as a polymerization initiator instead of camphorquinone.

TABLE 9-1
Composition of coating solutions prepared in Examples 31 to 36

| | | Example 31 | Example 32 | Example 33 | Example 34 | Example 35 | Example 36 |
|---|---|---|---|---|---|---|---|
| Polymerizable | Type | Polymerizable | Polymerizable | Polymerizable | Polymerizable | Polymerizable | Polymerizable |
| composition | | composition 3 | composition 3 | composition 3 | composition 3 | composition 3 | composition 3 |
| | Amount (g) | 1.26 | 1.20 | 1.09 | 1.26 | 1.20 | 1.09 |
| | Solid concentration | 80 | 80 | 80 | 80 | 80 | 80 |
| | (wt %) | | | | | | |
| | Total Concentration | 79.85 | 79.85 | 79.85 | 79.85 | 79.85 | 79.85 |
| | of [Compound (I) + | | | | | | |
| | Compound (II)] | | | | | | |
| | (wt %) | | | | | | |
| Solution of | Type | DS-Na-2 | DS-Na-2 | DS-Na-2 | DS-Na-2 | DS-Na-2 | DS-Na-2 |
| surfactant (III) | Amount (g) | 0.010 | 0.010 | 0.010 | 0.010 | 0.010 | 0.010 |
| | Solid concentration | 10 | 10 | 10 | 10 | 10 | 10 |
| | (wt %) | | | | | | |
| | Ratio with respect to | 0.10 | 0.10 | 0.11 | 0.10 | 0.10 | 0.11 |
| | [Compound (I) + | | | | | | |
| | Compound (II)] | | | | | | |
| | (wt %) | | | | | | |
| Polymerization | Type | Camphor- | Camphor- | Camphor- | LUCIRIN | LUCIRIN | LUCIRIN |
| initiator | | quinone | quinone | quinone | TPO | TPO | TPO |
| | Amount (g) | 0.022 | 0.068 | 0.132 | 0.022 | 0.068 | 0.132 |
| | Concentration with | 1.0 | 3.1 | 6.1 | 1.0 | 3.1 | 6.1 |
| | respect to total | | | | | | |
| | weight of coating | | | | | | |
| | solution (wt %) | | | | | | |
| | Ratio with respect to | 2.2 | 7.1 | 15.2 | 2.2 | 7.1 | 15.2 |
| | [Compound (I) + | | | | | | |
| | Compound (ID] | | | | | | |
| | (wt %) | | | | | | |
| Solvent | Ethanol (g) | 0.85 | 0.85 | 0.85 | 0.85 | 0.85 | 0.85 |
| | Distilled water (g) | 0.096 | 0.096 | 0.096 | 0.096 | 0.096 | 0.096 |
| Coating | Total Amount (g) | 2.238 | 2.224 | 2.178 | 2.238 | 2.224 | 2.178 |
| solufions | Total amount of | 1.031 | 1.029 | 1.005 | 1.031 | 1.029 | 1.005 |
| | solids (g) | | | | | | |
| | Solid concentration | 46 | 46 | 46 | 46 | 46 | 46 |
| | (wt %) | | | | | | |
| Evaluation of | Appearance | Transparent | Transparent | Yellow | Transparent | Transparent | Transparent |
| monolayer | Water contact | 13 | 11 | 12 | 16 | 10 | 10 |
| | angle (°) | | | | | | |

TABLE 9-2
Composition of coating solutions prepared in Examples 37 to 42

| | | Example 37 | Example 38 | Example 39 | Example 40 | Example 41 | Example 42 |
|---|---|---|---|---|---|---|---|
| Polymerizable | Type | Polymerizable | Polymerizable | Polymerizable | Polymerizable | Polymerizable | Polymerizable |
| composition | | composition 3 | composition 3 | composition 3 | composition 3 | composition 3 | composition 3 |
| | Amount (g) | 1.26 | 1.20 | 1.09 | 1.26 | 1.20 | 1.09 |
| | Solid concentration | 80 | 80 | 80 | 80 | 80 | 80 |
| | (wt %) | | | | | | |
| | Concentration of | 79.85 | 79.85 | 79.85 | 79.85 | 79.85 | 79.85 |
| | [Compound (I) + | | | | | | |
| | Compound (II)] | | | | | | |
| | Total (wt %) | | | | | | |
| Solution of | Type | DT-Na | DT-Na | DT-Na | DT-Na | DT-Na | DT-No |
| surfactant (III) | Amount (g) | 0.010 | 0.010 | 0.010 | 0.010 | 0.010 | 0.010 |
| | Solid concentration | 10 | 10 | 10 | 10 | 10 | 10 |
| | (wt %) | | | | | | |
| | Ratio with respect | 0.10 | 0.10 | 0.11 | 0.10 | 0.10 | 0.11 |
| | to [Compound (I) + | | | | | | |
| | Compound (II)] | | | | | | |
| | (wt %) | | | | | | |
| Polymerization | Type | Camphor- | Camphor- | Camphor- | LUCIRIN | LUCIRIN | LUCIRIN |
| initiator | | quinone | quinone | quinone | TPO | TPO | TPO |
| | Amount (g) | 0.022 | 0.068 | 0.132 | 0.022 | 0.068 | 0.132 |
| | Concentration with | 1.0 | 3.1 | 6.1 | 1.0 | 3.1 | 6.1 |
| | respect to total | | | | | | |
| | weight of coating | | | | | | |
| | solution (wt %) | | | | | | |
| | Ratio with respect | 2.2 | 7.1 | 15.2 | 2.2 | 7.1 | 15.2 |
| | to [Compound (I) + | | | | | | |
| | Compound (II)] | | | | | | |
| | (wt %) | | | | | | |
| Solvent | Ethanol (g) | 0.85 | 0.85 | 0.85 | 0.85 | 0.85 | 0.85 |
| | Distilled water (g) | 0.096 | 0.096 | 0.096 | 0.096 | 0.096 | 0.096 |
| Coating | Total Amount (g) | 2.238 | 2.224 | 2.178 | 2.238 | 2.224 | 2.178 |
| solutions | Total amount of | 1.031 | 1.029 | 1.005 | 1.031 | 1.029 | 1.005 |
| | solids (g) | | | | | | |
| | Solid concentration | 46 | 46 | 46 | 46 | 46 | 46 |
| | (wt %) | | | | | | |
| Evaluation of | Appearance | Transparent | Transparent | Yellow | Transparent | Transparent | Transparent |
| monolayer | Water contact | 15 | 13 | 12 | 14 | 8 | 9 |
| | angle (°) | | | | | | |

### Example 43

A quantity of 1.25 g of the polymerizable composition 3 obtained in Preparation Example 3 and having a solid concentration of 80 wt %, 0.010 g of the solution of DS-Na-2 (the solution of the surfactant (III)) obtained in Preparation Example 4-2 and having a solid concentration of 10 wt %, a mixed solvent of 0.85 g of ethanol and 0.096 g of distilled water as a diluting solvent, and 0.034 g (1.5 wt % with respect to the total weight) of camphorquinone (manufactured by Tokyo Chemical Industry Co., Ltd.) as a photopolymerization initiator were mixed, to give a coating solution having a solid concentration of 46 wt %.

A transparent acrylic plate (CLAREX-001; manufactured by Nitto Jushi Kogyo Co., Ltd.) pretreated in advance according to the method described in the section of the “Pretreatment of substrate” was used as a substrate, and the coating solution obtained above was coated on the surface of the substrate using a bar coater #30. The resultant was placed in a hot air dryer controlled at 50 to 60° C. for 5 minutes, to remove the solvent contained in the coating.

After sufficiently removing the solvent, the sample was placed in a visible light irradiation apparatus, Alpha light V (manufactured by MORITA Corporation, LED lamps; 400 to 408 nm, 465 to 475 nm: intensity of illumination: 60 mW/cm², accumulated light dose: 3600 mJ/cm², measured with UIT-250 (405 nm) manufactured by USHIO INC.), and irradiation was carried out for 1 minute, to form a hydrophilic monolayer film having a thickness of 18 μm on the transparent acrylic plate. The surface of the monolayer film was then washed with running water, followed by drying, and the appearance and the water contact angle of the resulting sample were evaluated. The results are shown in Table 10.

### Examples 44 to 51

The same operation as in Example 43 was carried out except that the polymerization initiator in Example 43 was replaced by 0.034 g (1.5 wt % with respect to the total weight) of each of the initiators shown in Table 10. In each of the Examples, the thus prepared coating solution having a solid concentration of 46 wt % was coated on a transparent acrylic plate in the same manner as in Example 43, followed by solvent removal and visible light irradiation, to form a monolayer film having a film thickness of 18 μm on the transparent acrylic plate. The surface of each of the monolayer films was then washed with running water, followed by drying, and the appearance and the water contact angle of each of the resulting samples were evaluated. The results are shown in Table 10.

### Example 52

A quantity of 1.20 g of the polymerizable composition 3 obtained in Preparation Example 3 and having a solid concentration of 80 wt %, 0.010 g of the solution of DS-Na-2 (the solution of the compound III) obtained in Preparation Example 4-2 and having a solid concentration of 10 wt %, a mixed solvent of 0.85 g of ethanol and 0.096 g of distilled water as a diluting solvent, and 0.034 g (1.5 wt % with respect to the total weight) of camphorquinone (manufactured by Wako Pure Chemical Industries, Ltd.) and 0.034 g (1.5 wt % with respect to the total weight) of N,N-dimethyl-p-toluidine (manufactured by Wako Pure Chemical Industries, Ltd.) as photopolymerization initiators were mixed, to give a coating solution having a solid concentration of 46 wt %.

A transparent acrylic plate (CLAREX-001; manufactured by Nitto Jushi Kogyo Co., Ltd.) pretreated in advance according to the method described in the section of the “Pretreatment of substrate” was used as a substrate, and the coating solution obtained above was coated on the surface of the substrate using a bar coater #30. The resultant was placed in a hot air dryer controlled at 50 to 60° C. for 5 minutes, to remove the solvent contained in the coating.

After sufficiently removing the solvent, the sample was placed in a visible light irradiation apparatus, Alpha light V (manufactured by MORITA Corporation, LED lamps; 400 to 408 nm, 465 to 475 nm: intensity of illumination: 60 mW/cm², accumulated light dose: 3600 mJ/cm², measured with UIT-250 (405 nm) manufactured by USHIO INC.), and irradiation was carried out for 1 minute, to form a hydrophilic monolayer film having a thickness of 18 μm on the transparent acrylic plate. The surface of the monolayer film was then washed with running water, followed by drying, and the appearance and the water contact angle of the resulting sample were evaluated. The results are shown in Table 10.

### Example 53

The same operation as in Example 52 was carried out except that the polymerization initiators in Example 52 were replaced by 0.034 g (1.5 wt % with respect to the total weight) of 2-ethylanthraquinone ((manufactured by Yamamoto Chemicals Inc.) and 0.034 g (1.5 wt % with respect to the total weight) of N,N-dimethyl-p-toluidine (manufactured by Wako Pure Chemical Industries, Ltd.). The thus prepared coating solution having a solid concentration of 46 wt % was coated on a transparent acrylic plate in the same manner as in Example 52, followed by solvent removal and visible light irradiation, to form a monolayer film having a film thickness of 18 μm on the transparent acrylic plate. The surface of the monolayer film was then washed with running water, followed by drying, and the appearance and the water contact angle of the resulting sample were evaluated. The results are shown in Table 10.

TABLE 10
Evaluation results for coating solutions prepared in
Examples 43 to 53

| | | | Water contact |
|---|---|---|---|
| | Type of initiator | Appearance | angle (°) |
| Example | Camphorquinone | Transparent | 16 |
| 43 | | | |
| Example | Speedcure CPTX | Brown | 20 |
| 44 | | | |
| Example | Speedcure DETX | Yellow | 25 |
| 45 | | | |
| Example | Speedcure CTX | Transparent | 30 |
| 46 | | | |
| Example | Speedcure ITX | Yellow | 27 |
| 47 | | | |
| Example | Irgacure 379EG | Transparent | 26 |
| 48 | | | |
| Example | Irgacure 1800 | Transparent | 26 |
| 49 | | | |
| Example | 2-ethylanthraquinone | Transparent | 21 |
| 50 | | | |
| Example | LUCIRIN TPO | Transparent | 12 |
| 51 | | | |
| Example | Camphorquinone | Yellow | 19 |
| 52 | N,N-dimethyl-p-toluidine | | |
| Example | 2-ethylanthraquinone | Red | 23 |
| 53 | N,N-dimethyl-p-toluidine | | |

### Example 54

A quantity of 1.25 g of the polymerizable composition 3 obtained in Preparation Example 3 and having a solid concentration of 80 wt %, 0.010 g of the solution of DS-Na-2 (the solution of the surfactant (III)) obtained in Preparation Example 4-2 and having a solid concentration of 10 wt %, a mixed solvent of 1.14 g of ethanol and 0.15 g of distilled water as a diluting solvent, and 0.030 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 40 wt %.

The thus obtained solution was coated on a transparent acrylic plate (CLAREX-001; manufactured by Nitto Jushi Kogyo Co., Ltd.) pretreated in advance according to the method described in the section of the “Pretreatment of substrate” using a brush for use in dentistry. The resultant was placed in a hot air dryer controlled at 50 to 60° C. for 5 minutes, to remove the solvent contained in the coating. The acrylic plate after drying was passed through a UV conveyor (high pressure mercury lamp, 160 W/cm, height: 14 cm, conveyor speed: 5 m/min, intensity of illumination: 400 mW/cm², and accumulated light dose: 1200 mJ/cm²; measured with UIT-250 manufactured by USHIO INC.), to form a hydrophilic monolayer film on the acrylic plate. The surface of the monolayer film was then washed with running water, followed by drying, and the appearance and the water contact angle of the resulting sample were evaluated. The results are shown in Table 11.

### Examples 55 to 57

The same operation as in Example 54 was carried out, except that the type of the solution of the surfactant (III) and the type of the solvent in Example 54 were changed to those shown in Table 11, to give coating solutions each having a solid concentration shown in Table 11. In each of Examples, the thus prepared coating solution was coated on a transparent acrylic plate in the same manner as in Example 54, followed by solvent removal and UV irradiation, to form a hydrophilic monolayer film on the transparent acrylic plate. The surface of each of the monolayer films was then washed with running water, followed by drying, and the appearance and the water contact angle of each of the resulting samples were evaluated. The results are shown in Table 11.

In Table 11, “DT-Na” in the section of the solution of the surfactant (III) indicates that the solution of DT-Na obtained in Preparation Example 4-3 and having a solid concentration of 10 wt % was used as the solution of the surfactant (III).

TABLE 11
Composition of coating solutions prepared in Examples 54 to 57

| | | Example 54 | Example 55 | Example 56 | Example 57 |
|---|---|---|---|---|---|
| Polymerizable | Type | Polymerizable | Polymerizable | Polymerizable | Polymerizable |
| composition | | composition 3 | composition 3 | composition 3 | composition 3 |
| | Amount (g) | 1.25 | 1.25 | 1.25 | 1.25 |
| | Solid concentration | 80 | 80 | 80 | 80 |
| | (wt %) | | | | |
| | Total Concentration of | 79.85 | 79.85 | 79.85 | 79.85 |
| | [Compound (I) + | | | | |
| | Compound (II)] (wt %) | | | | |
| Solution of | Type | DS-Na-2 | DT-Na | DS-Na-2 | DT-Na |
| surfactant (III) | Amount (g) | 0.010 | 0.010 | 0.010 | 0.010 |
| | Solid concentration | 10 | 10 | 10 | 10 |
| | (wt %) | | | | |
| | Ratio with respect to | 0.10 | 0.10 | 0.10 | 0.10 |
| | [Compound (I) + | | | | |
| | Compound (II)] (wt %) | | | | |
| Photopolymerization | Type | DAROCUR 1173 | DAROCUR 1173 | DAROCUR 1173 | DAROCUR 1173 |
| initiator | Amount (g) | 0.030 | 0.030 | 0.030 | 0.030 |
| | Ratio with respect to | 3.0 | 3.0 | 3.0 | 3.0 |
| | [Compound (I) + | | | | |
| | Compound (II)] (wt %) | | | | |
| Solvent | Ethanol (g) | 1.14 | 1.14 | 0 | 0 |
| | IPA (g) | 0 | 0 | 1.14 | 1.14 |
| | Distilled water (g) | 0.15 | 0.15 | 0.15 | 0.15 |
| Coating solutions | Total Amount (g) | 2.58 | 2.58 | 2.58 | 2.58 |
| | Total amount of solids | 1.031 | 1.031 | 1.031 | 1.031 |
| | (g) | | | | |
| | Solid concentration | 40 | 40 | 40 | 40 |
| | (wt %) | | | | |
| Substrate | Type | Acrylic plate | Acrylic plate | Acrylic plate | Acrylic plate |
| Evaluation of | Appearance | Transparent | Transparent | Transparent | Transparent |
| monolayer | Water contact angle | 8 | 6 | 12 | 13 |
| | (°) | | | | |

### <Preparation of Mouthpieces>

### (Obtaining of Impression and Bite)

Precise impressions of the upper jaw and the lower jaw of a patient with dentulous jaws were taken. Then a George gauge was used to obtain a bite at a position corresponding to 70% of the maximum protruded position of the lower jaw of the patient. Based on the thus obtained precise impressions, plaster figures consisting of an upper plaster figure and a lower plaster figure were prepared.

### (Preparation of Mouthpieces)

The plaster figures were fixed by the thus obtained bite, and mounted on an articulator. The bite was then removed, and the undercut portions of the plaster figures were blocked out by use of paraffin wax (manufactured by FEED Bextmill Co., Ltd.).

Next, a guide was prepared on the upper plaster figure using paraffin wax. The fixing parts for IST appliance (manufactured by Scheu Dental GmbH) used as a snoring prevention device were fixed on the buccal sides of the guide at positions between the first molars and the second molars.

Then an orthodontic resin material, Ortho Palette (manufactured by SHOFU Inc.) was built up on the plaster figure by sprinkle method. After removing the excessive resin, the resultant was placed in a pressure pot (manufactured by TOHO. Inc), and pressure polymerization was carried out for 10 minutes at 0.2 MPa in a hot water at a temperature of 40 to 50° C. After cooling, the resultant was taken out of the pressure pot, and an upper jaw mouthpiece was removed from the upper plaster figure, and the recontouring and polishing of the surfaces of the mouthpiece other than the surface thereof in contact with teeth were carried out.

For the lower plaster figure, a guide was prepared with paraffin wax in the same manner as described above, and mounted on the articulator again along with the above prepared upper jaw mouthpiece. Using positioning support for IST fixing parts (manufactured by Scheu Dental GmbH), the IST fixing parts for the lower jaw mouthpiece to be prepared were positioned forwardly of the IST fixing parts for the upper jaw mouthpiece, and fixed on the buccal sides of the guide. The resin was built up on the lower plaster figure so that the lower jaw mouthpiece to be prepared will engage with the upper jaw mouthpiece. Then pressure polymerization was carried out in the pressure pot under the same conditions as for the preparation of the upper jaw mouthpiece, followed by recontouring and polishing.

### <Pretreatment of Mouthpieces>

The polished mouthpieces obtained as described in the above mentioned “Preparation of mouthpieces” were immersed in IPA (isopropyl alcohol) for 5 minutes, retrieved from the IPA, and then subjected to air blowing. Then the mouthpieces were dried in a fan dryer controlled at 40° C. for 5 minutes, and the dried mouthpieces were used for the coating.

### Example 58

A quantity of 300 g of the polymerizable composition 3 obtained in Preparation Example 3 and having a solid concentration of 80 wt %, 2.4 g (0.1 wt % with respect to the total weight of the compound (I) and the compound (II)) of the solution of DS-Na-2 (the solution of the surfactant (III)) obtained in Preparation Example 4-2 and having a solid concentration of 10 wt %, a mixed solvent of 149.5 g of ethanol and 16.2 g of distilled water as a diluting solvent, and 7.2 g (3.0 wt % with respect to the total weight of the compound (I) and the compound (II)) of DAROCUR 1173 (manufactured by Ciba Specialty Chemicals Inc.) as a photopolymerization initiator were mixed, to give a coating solution having a solid concentration of 52 wt %.

The mouthpieces pretreated in advance according to the method described in the section of the “Pretreatment of mouthpieces” above were immersed in the thus obtained solution, and pulled up at 1 mm/sec, to coat the solution on the surfaces of the mouthpieces. The resulting mouthpieces were then placed in a hot air dryer controlled at 50 to 60° C. for 5 minutes, to remove the solvent contained in the coating. The mouthpieces after drying were subjected to UV irradiation by passing them through a UV conveyor (high pressure mercury lamp, 160 W/cm, height: 14 cm, conveyor speed: 5 m/min, intensity of illumination: 400 mW/cm², and accumulated light dose: 1200 mJ/cm²; measured with UIT-250 manufactured by USHIO INC.), and then by turning the mouthpieces over and passing them through the conveyor again so that the entire surfaces thereof were irradiated, to form a hydrophilic monolayer film on the surface of each of the mouthpieces.

### Examples 59 to 63

The same operation as in Example 58 was carried out, except that the type of the solution of the surfactant (III) and the amount of ethanol and distilled water in the diluting solvent in Example 58 were changed to those shown in the sections of Examples 59 to 63 in Table 12, to give coating solutions each having a solid concentration shown in Table 12. In each of Examples, the thus prepared coating solution was coated on the surfaces of the mouthpieces in the same manner as in Example 58, followed by solvent removal and UV irradiation, to form a monolayer film on the surface of each of the mouthpieces.

In Table 12, “DT-Na” in the section of the solution of the surfactant (III) indicates that the solution of DT-Na having a solid concentration of 10 wt % obtained in Preparation Example 4-3 was used as the solution of the surfactant (III).

### Comparative Example 4

The mouthpieces obtained in the same manner as described above were subjected to the following evaluations, without performing the coating with the dental composition of the invention.

### <Evaluation of Contamination Resistance 4>

The coated mouthpieces prepared in Examples 58 to 63 and the non-coated mouthpieces prepared in Comparative Example 4 were washed with water and dried. Then a 3 cm-mark was made on each of the mouthpieces on the biting surface of a molar teeth using a permanent marker, “Mackey ultrafine” manufactured by Zebra co., Ltd., (black, model number: MO-120-MC-BK), followed by washing with running water, 3 minutes after the marking. Most of the marks made on the coated mouthpieces prepared in Examples 58 to 63 were washed away with water, or able to be removed with a light rub.

On the other hand, the marks made on the uncoated mouthpieces prepared in Comparative Example 4 did not come off at all, even with rubbing.

TABLE 12
Composition of coating solutions prepared in Examples 58 to 63

| | | Example 58 | Example 59 | Example 60 | Example 61 | Example 62 | Example 63 |
|---|---|---|---|---|---|---|---|
| Polymerizable | Type | Polymerizable | Polymerizable | Polymerizable | Polymerizable | Polymerizable | Polymerizable |
| composition | | composition 3 | composition 3 | composition 3 | composition 3 | composition 3 | composition 3 |
| | Amount (g) | 300 | 300 | 300 | 300 | 300 | 300 |
| | Solid concentration | 80 | 80 | 80 | 80 | 80 | 80 |
| | (wt %) | | | | | | |
| | Concentration of | 79.85 | 79.85 | 79.85 | 79.85 | 79.85 | 79.85 |
| | [Compound (I) + | | | | | | |
| | Compound (II)] | | | | | | |
| | Total (wt %) | | | | | | |
| Solution of | Type | DS-Na-2 | DS-Na-2 | DS-Na-2 | DT-Na | DT-Na | DT-Na |
| surfactant (III) | Amount (g) | 2.4 | 2.4 | 2.4 | 2.4 | 2.4 | 2.4 |
| | Solid concentration | 10 | 10 | 10 | 10 | 10 | 10 |
| | (wt %) | | | | | | |
| | Ratio with respect | 0.10 | 0.10 | 0.10 | 0.10 | 0.10 | 0.10 |
| | to [Compound (I) + | | | | | | |
| | Compound (II)] | | | | | | |
| | (wt %) | | | | | | |
| Photo- | Type | DAROCUR | DAROCUR | DAROCUR | DAROCUR | DAROCUR | DAROCUR |
| polymerization | | 1173 | 1173 | 1173 | 1173 | 1173 | 1173 |
| initiator | Amount (g) | 7.2 | 7.2 | 7.2 | 7.2 | 7.2 | 7.2 |
| | Ratio with respect | 3.0 | 3.0 | 3.0 | 3.0 | 3.0 | 3.0 |
| | to [Compound (I) + | | | | | | |
| | Compound (ID] | | | | | | |
| | (wt %) | | | | | | |
| Solvent | Ethanol (g) | 149.5 | 205.3 | 277.8 | 149.5 | 205.3 | 277.8 |
| | Distilled water (g) | 16.2 | 22.4 | 30.4 | 16.2 | 22.4 | 30.4 |
| Coating | Total Amount (g) | 475.3 | 537.3 | 617.8 | 475.3 | 537.3 | 617.8 |
| solutions | Total amount of | 247.44 | 247.44 | 247.44 | 247.44 | 247.44 | 247.44 |
| | solids (g) | | | | | | |
| | Solid concentration | 52 | 46 | 40 | 52 | 46 | 40 |
| | (wt %) | | | | | | |

### INDUSTRIAL APPLICABILITY

A cured product obtainable from the dental composition according to the invention, such as a monolayer film, has a high hydrophilicity and antifouling properties, and thus it is useful in various types of dental applications. In particular, the cured product is useful as a dental coating material, especially, as a surface coating for a dental prosthesis.

1. A dental prosthesis comprising a monolayer film obtained by curing a composition comprising:
a compound (I) having at least one hydrophilic group selected from anionic hydrophilic groups and cationic hydrophilic groups, and at least one functional group with a polymerizable carbon-carbon double bond;
a compound (II) having two or more functional groups with a polymerizable carbon-carbon double bond (wherein the compound (II) has no anionic hydrophilic group nor cationic hydrophilic group); and
a surfactant (III) having a hydrophilic moiety including an anionic hydrophilic group, a cationic hydrophilic group or two or more hydroxyl groups, and a hydrophobic moiety composed of an organic residue (wherein the surfactant has no polymerizable carbon-carbon double bond).

2. The dental prosthesis according to claim 1, wherein the monolayer film has a concentration gradient (Sa/Da) of at least one type of hydrophilic groups selected from anionic hydrophilic groups, cationic hydrophilic groups and hydroxyl group, of 1.1 or more,
wherein the concentration gradient (Sa/Da) is obtained from:
the concentration (Sa) at the surface of the monolayer film; and
the concentration (Da) at a ½ point in the thickness of the monolayer film.

3. The dental prosthesis according to claim 1, wherein the monolayer film has a water contact angle of 30° or less.

4. The dental prosthesis according to claim 1, wherein the monolayer film has a film thickness of 0.1 to 100 μm.

5. The dental prosthesis according to claim 1, wherein the monolayer film is obtained by: coating a composition comprising the compound (I), the compound (II), the compound (III) and a solvent on a substrate; then removing the solvent; and then subjecting the resultant to curing.

6. The dental prosthesis according to claim 5, wherein the coating step is carried out by a dip method.

7. The dental prosthesis according to claim 1, wherein the compound (I) is a compound represented by the general formula (100) below: 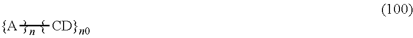
(wherein in the formula (100),
A represents a C₂₋₁₀₀ organic group having 1 to 5 functional groups with a polymerizable carbon-carbon double bond;
CD represents a group containing at least one hydrophilic group, selected from those groups represented by the general formulas (101), (102) and (112) below;
n represents the number of As bound to CD and is 1 or 2; and
n0 represents the number of CDs bound to A and is an integer of 1 to 5); 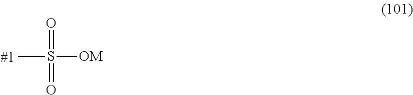
(wherein in the formula (101), M represents a hydrogen atom, an alkali metal, an alkaline earth metal (½ atom) or an ammonium ion; and #1 indicates a hand bound to a carbon atom present in A in the formula (100)); 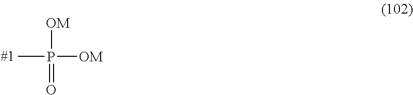
(wherein in the formula (102), M represents a hydrogen atom, an alkali metal, an alkaline earth metal (½ atom) or an ammonium ion; and #1 indicates a hand bound to a carbon atom present in A in the formula (100)); and 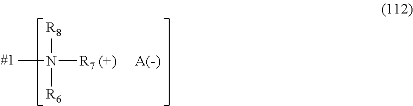
(wherein in the formula (112), A(−) represents a halogen ion, a formate ion, an acetate ion, a sulfate ion, a hydrogen sulfate ion, a phosphate ion or a hydrogen phosphate ion; R₆ to R₈ each independently represent a hydrogen atom, a C₁₋₂₀ alkyl, alkylaryl, alkylbenzyl, alkylcycloalkyl, alkylcycloalkylmethyl or cycloalkyl group, a phenyl group or a benzyl group; and #1 indicates a hand bound to a carbon atom present in A in the formula (100)).

8. The dental prosthesis according to claim 7, wherein A in the general formula (100) is at least one functional group selected from those groups represented by the general formulas (120), (123) and (124) below: 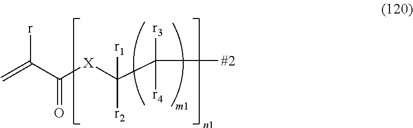
(wherein in the formula (120), X represents —O—, —S—, —NH— or —NCH₃—; r represents a hydrogen atom or a methyl group; r₁ to r₄ each independently represent a hydrogen atom, a methyl group, an ethyl group or a hydroxyl group; m1 is an integer of 0 to 10; n1 is an integer of 0 to 100; and #2 indicates a hand bound to #1 present in at least one group selected from those groups represented by the general formulas (101), (102) and (112); 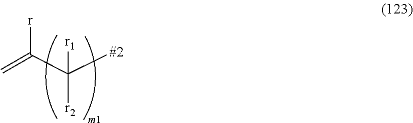
(wherein in the formula (123), r represents a hydrogen atom or a methyl group; r₁ and r2 each independently represent a hydrogen atom, a methyl group, an ethyl group or a hydroxyl group; m1 is an integer of 0 to 10; and #2 indicates a hand bound to #1 present in at least one group selected from those groups represented by the general formulas (101), (102) and (112)); and 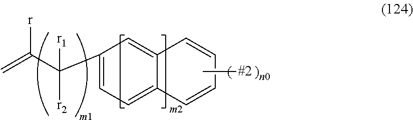
(wherein in the formula (124), r represents a hydrogen atom or a methyl group; r₁ and r₂ each independently represent a hydrogen atom, a methyl group, an ethyl group or a hydroxyl group; m1 is an integer of 0 to 10; m2 is an integer of 0 to 5; n0 is an integer of 1 to 5; and #2 indicates a hand bound to #1 present in at least one group selected from those groups represented by the general formulas (101), (102) and (112)).

9. The dental prosthesis according to claim 1, wherein the surfactant is a compound represented by the general formula (300) below: 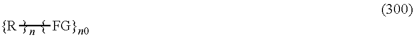
(wherein in the formula (300),
R represents a C₄₋₁₀₀ organic residue,
FG represents a group containing at least one hydrophilic group, selected from those groups represented by the general formulas (301), (302), (312) and (318) below;
n represents the number of Rs bound to FG and is 1 or 2; and
n0 represents the number of FGs bound to R and is an integer of 1 to 5; and when FG is a group including one hydroxyl group, n0 is an integer of 2 to 5); 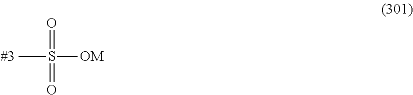
(wherein in the formula (301), M represents a hydrogen atom, an alkali metal, an alkaline earth metal (½ atom) or an ammonium ion; and #3 indicates a hand bound to a carbon atom present in R in the formula (300)); 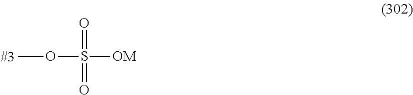
(wherein in the formula (302), M represents a hydrogen atom, an alkali metal, an alkaline earth metal (½ atom) or an ammonium ion; and #3 indicates a hand bound to a carbon atom present in R in the formula (300)); 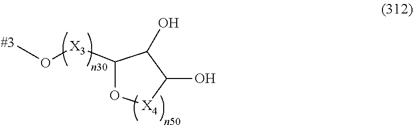
wherein in the formula (312), X₃ and X₄ each independently represent —CH₂—, —CH(OH)— or —CO—; n₃₀ is an integer of 0 to 3; n₅₀ is an integer of 0 to 5; when n₃₀ is 2 or greater, X₃s may be the same as or different from one another; when n₅₀ is 2 or greater, X₄s may be the same as or different from one another; and #3 indicates a hand bound to a carbon atom present in R in the formula (300); and 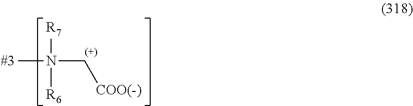
(wherein in the formula (318), R₆ and R₇ each independently represent a hydrogen atom, a C₁₋₂₀ alkyl, alkylaryl, alkylbenzyl, alkylcycloalkyl, alkylcycloalkylmethyl or cycloalkyl group, a phenyl group or a benzyl group; and #3 indicates a hand bound to a carbon atom present in R in the formula (300)).
